SEQUENCE LISTING (604 sequences)

SEQ ID NO: 1 - protein, 8 aa, synthetic construct
GLTFRNYD

SEQ ID NO: 2 - protein, 8 aa, synthetic construct
ASWSEEDT

SEQ ID NO: 3 - protein, 19 aa, synthetic construct
AAGLTVNGRLLTRTYEYDH

SEQ ID NO: 4 - protein, 8 aa, synthetic construct
GSISSIDH

SEQ ID NO: 5 - protein, 7 aa, synthetic construct
INSGGRT

SEQ ID NO: 6 - protein, 14 aa, synthetic construct
NVLLRDRSGSGRTY

SEQ ID NO: 7 - protein, 8 aa, synthetic construct
ERTAITYS

SEQ ID NO: 8 - protein, 8 aa, synthetic construct
ISGRDGRT

SEQ ID NO: 9 - protein, 15 aa, synthetic construct
ATSPLVSTDQPDFYS

SEQ ID NO: 10 - protein, 8 aa, synthetic construct
GSIFRSVP

SEQ ID NO: 11 - protein, 7 aa, synthetic construct
IFVDGST

SEQ ID NO: 12 - protein, 5 aa, synthetic construct
FMNGD

SEQ ID NO: 13 - protein, 8 aa, synthetic construct
ESRFSIKP

SEQ ID NO: 14 - protein, 7 aa, synthetic construct
ITSPGMA

SEQ ID NO: 15 - protein, 7 aa, synthetic construct
RDILSGS

SEQ ID NO: 16 - protein, 8 aa, synthetic construct
GRLLSDNS

SEQ ID NO: 17 - protein, 7 aa, synthetic construct
ITSGGGT

SEQ ID NO: 18 - protein, 7 aa, synthetic construct
NWPRYGD

SEQ ID NO: 19 - protein, 8 aa, synthetic construct
GRPFSSYA

SEQ ID NO: 20 - protein, 8 aa, synthetic construct
IGGDGSTT

SEQ ID NO: 21 - protein, 17 aa, synthetic construct
ALDFSLNRIVFGTRADY

SEQ ID NO: 22 - protein, 8 aa, synthetic construct
GRTFSTWH

SEQ ID NO: 23 - protein, 8 aa, synthetic construct
IGGSGGSR

SEQ ID NO: 24 - protein, 17 aa, synthetic construct
ATGPAAFGSRKGTSYDY

SEQ ID NO: 25 - protein, 8 aa, synthetic construct
GRAFRINS

SEQ ID NO: 26 - protein, 8 aa, synthetic construct
ISWSGRDT

SEQ ID NO: 27 - protein, 18 aa, synthetic construct
AARVFFDSGSYAASEYSN

SEQ ID NO: 28 - protein, 8 aa, synthetic construct
GDTFSNYA

SEQ ID NO: 29 - protein, 8 aa, synthetic construct
ISWHGGRA

SEQ ID NO: 30 - protein, 16 aa, synthetic construct
AARLLGGGWSGEEYDY

SEQ ID NO: 31 - protein, 8 aa, synthetic construct
GRTFSGTA

SEQ ID NO: 32 - protein, 8 aa, synthetic construct
IWWSRYAT

SEQ ID NO: 33 - protein, 16 aa, synthetic construct
AAGHRGYSRFAEAYDY

SEQ ID NO: 34 - protein, 8 aa, synthetic construct
ERTFGIRT

SEQ ID NO: 35 - protein, 8 aa, synthetic construct
IKWAGGNT

SEQ ID NO: 36 - protein, 19 aa, synthetic construct
AAAKVYYYTPTMGPGSYEF

SEQ ID NO: 37 - protein, 8 aa, synthetic construct
GRTGSHLD

SEQ ID NO: 38 - protein, 8 aa, synthetic construct
ISRDGFRI

SEQ ID NO: 39 - protein, 17 aa, synthetic construct
AADAAGFGSRFVSSYDY

SEQ ID NO: 40 - protein, 10 aa, synthetic construct
GRTFDSDRFG

SEQ ID NO: 41 - protein, 8 aa, synthetic construct
INWRGGGA

SEQ ID NO: 42 - protein, 17 aa, synthetic construct
VADIAAWGARSAASYEY

SEQ ID NO: 43 - protein, 8 aa, synthetic construct
RAISSRWP

SEQ ID NO: 44 - protein, 7 aa, synthetic construct
ISHGSIT

SEQ ID NO: 45 - protein, 11 aa, synthetic construct
YAEDWDTRVQY

SEQ ID NO: 46 - protein, 8 aa, synthetic construct
GLTFSSYT

SEQ ID NO: 47 - protein, 8 aa, synthetic construct
ASWSGGST

SEQ ID NO: 48 - protein, 15 aa, synthetic construct
AAEKAPSRTVAAYEY

SEQ ID NO: 49 - protein, 8 aa, synthetic construct
GRTSSGYA

SEQ ID NO: 50 - protein, 8 aa, synthetic construct
IAWSAGST

SEQ ID NO: 51 - protein, 16 aa, synthetic construct
AAGTRMPSRMTNAYDY

SEQ ID NO: 52 - protein, 8 aa, synthetic construct
GRTSSGYA

SEQ ID NO: 53 - protein, 8 aa, synthetic construct
ISWSGGST

SEQ ID NO: 54 - protein, 16 aa, synthetic construct
AAGTRQVSQTTQAYDY

SEQ ID NO: 55 - protein, 8 aa, synthetic construct
GRPFSSYI

SEQ ID NO: 56 - protein, 8 aa, synthetic construct
INWSGDHT

SEQ ID NO: 57 - protein, 23 aa, synthetic construct
AAKLSAGSSTDTVLHNNRWSWDS

SEQ ID NO: 58 - protein, 8 aa, synthetic construct
GGTYTTWT

SEQ ID NO: 59 - protein, 8 aa, synthetic construct
IRRTGGDP

SEQ ID NO: 60 - protein, 15 aa, synthetic construct
AASPLWTSSQDDYRH

SEQ ID NO: 61 - protein, 8 aa, synthetic construct
GAYLIVSD

SEQ ID NO: 62 - protein, 6 aa, synthetic construct
IGRGGT

SEQ ID NO: 63 - protein, 5 aa, synthetic construct
NIVDY

SEQ ID NO: 64 - protein, 8 aa, synthetic construct
GFTFSSYW

SEQ ID NO: 65 - protein, 8 aa, synthetic construct
MSPEGDMT

SEQ ID NO: 66 - protein, 18 aa, synthetic construct
VKGRTHGSGLYGARDFES

SEQ ID NO: 67 - protein, 8 aa, synthetic construct
GYISSTHF

SEQ ID NO: 68 - protein, 7 aa, synthetic construct
ITGSDLT

SEQ ID NO: 69 - protein, 11 aa, synthetic construct
RLWGLGLGDGY

SEQ ID NO: 70 - protein, 8 aa, synthetic construct
GFTLSDYW

SEQ ID NO: 71 - protein, 8 aa, synthetic construct
IKAGDDTT

SEQ ID NO: 72 - protein, 16 aa, synthetic construct
ARSPYGTYRLDRRYDF

SEQ ID NO: 73 - protein, 8 aa, synthetic construct
GSSSSINA

SEQ ID NO: 74 - protein, 9 aa, synthetic construct
ISRARGDST

SEQ ID NO: 75 - protein, 13 aa, synthetic construct
YVAGDRSLDFRSY

SEQ ID NO: 76 - protein, 8 aa, synthetic construct
GTISSTDF

SEQ ID NO: 77 - protein, 7 aa, synthetic construct
ITGSDLT

SEQ ID NO: 78 - protein, 9 aa, synthetic construct
RVWGLGYYY

SEQ ID NO: 79 - protein, 8 aa, synthetic construct
GRTFDYHA

SEQ ID NO: 80 - protein, 8 aa, synthetic construct
IGGSGGSR

SEQ ID NO: 81 - protein, 17 aa, synthetic construct
ATGPAGYGSRKSTSYDY

SEQ ID NO: 82 - protein, 8 aa, synthetic construct
EEWFRINN

SEQ ID NO: 83 - protein, 7 aa, synthetic construct
ITPSGST

SEQ ID NO: 84 - protein, 7 aa, synthetic construct
RDISGGS

SEQ ID NO: 85 - protein, 13 aa, synthetic construct
GRTLTDRTLTDYA

SEQ ID NO: 86 - protein, 8 aa, synthetic construct
IRWSDYRT

SEQ ID NO: 87 - protein, 16 aa, synthetic construct
VAGHRLNSRFAEAYNY

SEQ ID NO: 88 - protein, 8 aa, synthetic construct
ERPFSSYA

SEQ ID NO: 89 - protein, 8 aa, synthetic construct
IGGDGSIT

SEQ ID NO: 90 - protein, 17 aa, synthetic construct
ALDFSFNRIVLGSRADY

SEQ ID NO: 91 - protein, 6 aa, synthetic construct
GRTFNA

SEQ ID NO: 92 - protein, 8 aa, synthetic construct
IRWNGYMT

SEQ ID NO: 93 - protein, 16 aa, synthetic construct
AAGDRGSSRFVAAYDY

SEQ ID NO: 94 - protein, 8 aa, synthetic construct
GFTFDDYA

SEQ ID NO: 95 - protein, 8 aa, synthetic construct
ISSSRATT

SEQ ID NO: 96 - protein, 16 aa, synthetic construct
AAGTRMPSRMTNAYDY

SEQ ID NO: 97 - protein, 8 aa, synthetic construct
RAISSRWP

SEQ ID NO: 98 - protein, 7 aa, synthetic construct
ISHGSIT

SEQ ID NO: 99 - protein, 13 aa, synthetic construct
YAAGQSTAPSASY

SEQ ID NO: 100 - protein, 6 aa, synthetic construct
VRTLTA

SEQ ID NO: 101 - protein, 8 aa, synthetic construct
MRWSDGST

SEQ ID NO: 102 - protein, 17 aa, synthetic construct
AADQVIFYSRKPTDYDY

SEQ ID NO: 103 - protein, 8 aa, synthetic construct
GSISSFNA

SEQ ID NO: 104 - protein, 7 aa, synthetic construct
ITGSGST

SEQ ID NO: 105 - protein, 20 aa, synthetic construct
YADLSTYNAAWNGGVYRNNY

SEQ ID NO: 106 - protein, 8 aa, synthetic construct
GLPFSTYF

SEQ ID NO: 107 - protein, 8 aa, synthetic construct
IGGNGGSR

SEQ ID NO: 108 - protein, 17 aa, synthetic construct
ATGPAAFGSRKTSSYDY

SEQ ID NO: 109 - protein, 8 aa, synthetic construct
ESRFSSKP

SEQ ID NO: 110 - protein, 7 aa, synthetic construct
ITSPGMA

SEQ ID NO: 111 - protein, 7 aa, synthetic construct
RDILSDS

SEQ ID NO: 112 - protein, 8 aa, synthetic construct
GRSFSTYF

SEQ ID NO: 113 - protein, 8 aa, synthetic construct
IGGNGGSR

SEQ ID NO: 114 - protein, 17 aa, synthetic construct
ATGPRGFGSRKSTSYDY

SEQ ID NO: 115 - protein, 8 aa, synthetic construct
GFTLSDYW

SEQ ID NO: 116 - protein, 8 aa, synthetic construct
IASGGSDT

SEQ ID NO: 117 - protein, 16 aa, synthetic construct
ARSPYGTYRLDRRYDT

SEQ ID NO: 118 - protein, 8 aa, synthetic construct
GYISSTHF

SEQ ID NO: 119 - protein, 7 aa, synthetic construct
ITGSDLT

SEQ ID NO: 120 - protein, 11 aa, synthetic construct
RLWGLGLGDGY

SEQ ID NO: 121 - protein, 8 aa, synthetic construct
GSIPSTHF

SEQ ID NO: 122 - protein, 7 aa, synthetic construct
ITGSDLT

SEQ ID NO: 123 - protein, 9 aa, synthetic construct
RVWGVGYDY

SEQ ID NO: 124 - protein, 8 aa, synthetic construct
GRPFSSYA

SEQ ID NO: 125 - protein, 8 aa, synthetic construct
IGGDGSTT

SEQ ID NO: 126 - protein, 17 aa, synthetic construct
ALDFSLNRIVFGTRADY

SEQ ID NO: 127 - protein, 8 aa, synthetic construct
GRLISADS

SEQ ID NO: 128 - protein, 7 aa, synthetic construct
ITSGGGT

SEQ ID NO: 129 - protein, 7 aa, synthetic construct
HWPRYGD

SEQ ID NO: 130 - protein, 8 aa, synthetic construct
GLTFSSYS

SEQ ID NO: 131 - protein, 8 aa, synthetic construct
ISWSNGRT

SEQ ID NO: 132 - protein, 15 aa, synthetic construct
AAEKAPSRTVAAYEY

SEQ ID NO: 133 - protein, 8 aa, synthetic construct
GSSSSINA

SEQ ID NO: 134 - protein, 9 aa, synthetic construct
ISRARGDST

SEQ ID NO: 135 - protein, 13 aa, synthetic construct
YVAGNRDLDFRSY

SEQ ID NO: 136 - protein, 8 aa, synthetic construct
GSISSTDF

SEQ ID NO: 137 - protein, 7 aa, synthetic construct
ITGSDLT

SEQ ID NO: 138 - protein, 9 aa, synthetic construct
RVWGLGYAY

SEQ ID NO: 139 - protein, 8 aa, synthetic construct
GLTFSSYS

SEQ ID NO: 140 - protein, 8 aa, synthetic construct
ISWSGGRT

SEQ ID NO: 141 - protein, 15 aa, synthetic construct
AAERAPSRQVAAYEF

SEQ ID NO: 142 - protein, 8 aa, synthetic construct
GRTVSNYA

SEQ ID NO: 143 - protein, 8 aa, synthetic construct
VAWTGGRT

SEQ ID NO: 144 - protein, 16 aa, synthetic construct
AARLLGGGWSGEEYDS

SEQ ID NO: 145 - protein, 8 aa, synthetic construct
RSISSRWP

SEQ ID NO: 146 - protein, 7 aa, synthetic construct
ISHGSII

SEQ ID NO: 147 - protein, 11 aa, synthetic construct
YAEDWDTRVQY

SEQ ID NO: 148 - protein, 8 aa, synthetic construct
GSIPSTHF

SEQ ID NO: 149 - protein, 7 aa, synthetic construct
ITGSDLT

SEQ ID NO: 150 - protein, 9 aa, synthetic construct
RVWGVGYDY

SEQ ID NO: 151 - protein, 8 aa, synthetic construct
ERTFSTYF

SEQ ID NO: 152 - protein, 8 aa, synthetic construct
IGGNGGSR

SEQ ID NO: 153 - protein, 17 aa, synthetic construct
ATGPRGFGSRKSTSYDY

SEQ ID NO: 154 - protein, 8 aa, synthetic construct
GRIFSTYS

SEQ ID NO: 155 - protein, 8 aa, synthetic construct
IGGSGGSR

SEQ ID NO: 156 - protein, 17 aa, synthetic construct
ATGPRGFGSRKTTSYDY

SEQ ID NO: 157 - protein, 8 aa, synthetic construct
GRTVSSYA

SEQ ID NO: 158 - protein, 8 aa, synthetic construct
MSWTGGRT

SEQ ID NO: 159 - protein, 16 aa, synthetic construct
AGRLLGGGWSGEEYGY

SEQ ID NO: 160 - protein, 8 aa, synthetic construct
GRAFSTYF

SEQ ID NO: 161 - protein, 8 aa, synthetic construct
IGGNGGSR

SEQ ID NO: 162 - protein, 17 aa, synthetic construct
ETGPRGFGSRKNISYDY

SEQ ID NO: 163 - protein, 8 aa, synthetic construct
GLTFSSYS

SEQ ID NO: 164 - protein, 8 aa, synthetic construct
ISWSGGRT

SEQ ID NO: 165 - protein, 15 aa, synthetic construct
AAERAPSRQVAAYEY

SEQ ID NO: 166 - protein, 8 aa, synthetic construct
GFTFSTYS

SEQ ID NO: 167 - protein, 8 aa, synthetic construct
ISWSNGRT

SEQ ID NO: 168 - protein, 15 aa, synthetic construct
AAEKAPSRKVSAYEY

SEQ ID NO: 169 - protein, 8 aa, synthetic construct
ERTFSTYF

SEQ ID NO: 170 - protein, 8 aa, synthetic construct
IGGNGGSR

SEQ ID NO: 171 - protein, 17 aa, synthetic construct
ATGPRGFGSRKSTSYDY

SEQ ID NO: 172 - protein, 8 aa, synthetic construct
GLTFSSYS

SEQ ID NO: 173 - protein, 8 aa, synthetic construct
ISWSGGRT

SEQ ID NO: 174 - protein, 15 aa, synthetic construct
AAERAPSRQVAAYEF

SEQ ID NO: 175 - protein, 8 aa, synthetic construct
GSIPSTHF

SEQ ID NO: 176 - protein, 7 aa, synthetic construct
ITGSDLT

SEQ ID NO: 177 - protein, 9 aa, synthetic construct
RVWGVGYDY

SEQ ID NO: 178 - protein, 8 aa, synthetic construct
GRTTSTYF

SEQ ID NO: 179 - protein, 8 aa, synthetic construct
IGGSGGSR

SEQ ID NO: 180 - protein, 17 aa, synthetic construct
ATGPRGFGSRKSTSYDY

SEQ ID NO: 181 - protein, 8 aa, synthetic construct
GSIPSTHF

SEQ ID NO: 182 - protein, 7 aa, synthetic construct
ITGSDLT

SEQ ID NO: 183 - protein, 9 aa, synthetic construct
RVWGVGYDY

SEQ ID NO: 184 - protein, 8 aa, synthetic construct
GGTFSSYA

SEQ ID NO: 185 - protein, 8 aa, synthetic construct
IAWHGGRT

SEQ ID NO: 186 - protein, 16 aa, synthetic construct
AARLLGGGWSGEEYEY

SEQ ID NO: 187 - protein, 8 aa, synthetic construct
GSSSSINA

SEQ ID NO: 188 - protein, 9 aa, synthetic construct
ISRARGDST

SEQ ID NO: 189 - protein, 13 aa, synthetic construct
YVAGDRSLDFRSY

SEQ ID NO: 190 - protein, 8 aa, synthetic construct
GRSFSTYF

SEQ ID NO: 191 - protein, 8 aa, synthetic construct
IGGNGGSR

SEQ ID NO: 192 - protein, 17 aa, synthetic construct
ATGPRGFGSRKSTSYDY

SEQ ID NO: 193 - protein, 8 aa, synthetic construct
GRTVSTYF

SEQ ID NO: 194 - protein, 8 aa, synthetic construct
IGGSGGSR

SEQ ID NO: 195 - protein, 17 aa, synthetic construct
ATGPAGFGSRKSTSYDY

SEQ ID NO: 196 - protein, 8 aa, synthetic construct
GLTFSSYS

SEQ ID NO: 197 - protein, 8 aa, synthetic construct
ISWSGGRT

SEQ ID NO: 198 - protein, 15 aa, synthetic construct
AAERAPSRQVAAYEY

SEQ ID NO: 199 - protein, 8 aa, synthetic construct
GSIASTHF

SEQ ID NO: 200 - protein, 7 aa, synthetic construct
LTESSLT

SEQ ID NO: 201 - protein, 9 aa, synthetic construct
GVWGLGFAY

SEQ ID NO: 202 - protein, 8 aa, synthetic construct
ERTFDTYT

SEQ ID NO: 203 - protein, 8 aa, synthetic construct
IGGSGGSR

SEQ ID NO: 204 - protein, 17 aa, synthetic construct
ATGPAGFGSRKTTSYDY

SEQ ID NO: 205 - protein, 8 aa, synthetic construct
GLTFSSYS

SEQ ID NO: 206 - protein, 8 aa, synthetic construct
ISWSAGRT

SEQ ID NO: 207 - protein, 15 aa, synthetic construct
AAERAPSRQVAAYEY

SEQ ID NO: 208 - protein, 8 aa, synthetic construct
GLTFSSYS

SEQ ID NO: 209 - protein, 8 aa, synthetic construct
ISWSAGRT

SEQ ID NO: 210 - protein, 15 aa, synthetic construct
AAERAPSRQVAAYEY

SEQ ID NO: 211 - protein, 8 aa, synthetic construct
RSISSRWP

SEQ ID NO: 212 - protein, 7 aa, synthetic construct
ISHGSII

SEQ ID NO: 213 - protein, 11 aa, synthetic construct
YAEDWDTRVQY

SEQ ID NO: 214 - protein, 8 aa, synthetic construct
GRPFSSYA

SEQ ID NO: 215 - protein, 8 aa, synthetic construct
IGGDGSTT

SEQ ID NO: 216 - protein, 17 aa, synthetic construct
ALDFSLNRIVFGTRADY

SEQ ID NO: 217 - protein, 8 aa, synthetic construct
GLTFSSYS

SEQ ID NO: 218 - protein, 8 aa, synthetic construct
ISWSGGRT

SEQ ID NO: 219 - protein, 15 aa, synthetic construct
AAERAPSRQVAAYEF

SEQ ID NO: 220 - protein, 8 aa, synthetic construct
GDRFSIKP

SEQ ID NO: 221 - protein, 7 aa, synthetic construct
ITSPGTA

SEQ ID NO: 222 - protein, 7 aa, synthetic construct
RDILSDS

SEQ ID NO: 223 - protein, 8 aa, synthetic construct
GRTFSNYD

SEQ ID NO: 224 - protein, 9 aa, synthetic construct
CSWGGENTA

SEQ ID NO: 225 - protein, 19 aa, synthetic construct
VAGLTVNGRLLTRTYEYDN

SEQ ID NO: 226 - protein, 8 aa, synthetic construct
GRTFSSTA

SEQ ID NO: 227 - protein, 8 aa, synthetic construct
IRWSDYRT

SEQ ID NO: 228 - protein, 16 aa, synthetic construct
VAGHRLNSRFAEAYDY

SEQ ID NO: 229 - protein, 8 aa, synthetic construct
GRAFSTYH

SEQ ID NO: 230 - protein, 8 aa, synthetic construct
IGGSGGSR

SEQ ID NO: 231 - protein, 17 aa, synthetic construct
ATGPAGFGSRRSTSYDY

SEQ ID NO: 232 - protein, 8 aa, synthetic construct
GSISSTHF

SEQ ID NO: 233 - protein, 7 aa, synthetic construct
ITESSLT

SEQ ID NO: 234 - protein, 9 aa, synthetic construct
GVWGLGYAY

SEQ ID NO: 235 - protein, 8 aa, synthetic construct
GRTTSTYF

SEQ ID NO: 236 - protein, 8 aa, synthetic construct
IGGSGGSR

SEQ ID NO: 237 - protein, 17 aa, synthetic construct
ATGPRGFGSRKSTSYDY

SEQ ID NO: 238 - protein, 8 aa, synthetic construct
GSRFSTKP

SEQ ID NO: 239 - protein, 7 aa, synthetic construct
ITTPGMA

SEQ ID NO: 240 - protein, 7 aa, synthetic construct
RDILSDD

SEQ ID NO: 241 - protein, 8 aa, synthetic construct
GRSFSTYF

SEQ ID NO: 242 - protein, 8 aa, synthetic construct
IGGNGGSR

SEQ ID NO: 243 - protein, 17 aa, synthetic construct
ATGPRGFGSRKSTSYDY

SEQ ID NO: 244 - protein, 8 aa, synthetic construct
GRTFSSTA

SEQ ID NO: 245 - protein, 8 aa, synthetic construct
IRWSDYRT

SEQ ID NO: 246 - protein, 16 aa, synthetic construct
VAGHRLNSRFAEAYDY

SEQ ID NO: 247 - protein, 8 aa, synthetic construct
GLPFSTYF

SEQ ID NO: 248 - protein, 8 aa, synthetic construct
IGGNGGSR

SEQ ID NO: 249 - protein, 17 aa, synthetic construct
ATGPAAFGSRKTSSYDY

SEQ ID NO: 250 - protein, 8 aa, synthetic construct
GRSFSTDF

SEQ ID NO: 251 - protein, 8 aa, synthetic construct
IGGNGGSR

SEQ ID NO: 252 - protein, 17 aa, synthetic construct
ATGPRGFGSRKSTSYDY

SEQ ID NO: 253 - protein, 8 aa, synthetic construct
GRLISANS

SEQ ID NO: 254 - protein, 7 aa, synthetic construct
ITSGGGT

SEQ ID NO: 255 - protein, 7 aa, synthetic construct
NWPRYGD

SEQ ID NO: 256 - protein, 8 aa, synthetic construct
GLTFSSYS

SEQ ID NO: 257 - protein, 8 aa, synthetic construct
ISWSAGRT

SEQ ID NO: 258 - protein, 15 aa, synthetic construct
AAERAPSRRVDAYEY

SEQ ID NO: 259 - protein, 8 aa, synthetic construct
GRSFSTYF

SEQ ID NO: 260 - protein, 8 aa, synthetic construct
IGGNGGSR

SEQ ID NO: 261 - protein, 17 aa, synthetic construct
ATGPRGFGSRKSTSYDY

SEQ ID NO: 262 - protein, 8 aa, synthetic construct
GLTFSSYS

SEQ ID NO: 263 - protein, 8 aa, synthetic construct
ISWSGGRT

SEQ ID NO: 264 - protein, 15 aa, synthetic construct
AAERAPSRQVAAYEF

SEQ ID NO: 265 - protein, 8 aa, synthetic construct
GLTFSSYS

SEQ ID NO: 266 - protein, 8 aa, synthetic construct
ISWSYGST

SEQ ID NO: 267 - protein, 15 aa, synthetic construct
AAEKAPSRRVAAYEY

SEQ ID NO: 268 - protein, 8 aa, synthetic construct
GRSFSTYF

SEQ ID NO: 269 - protein, 8 aa, synthetic construct
IGGNGGSR

SEQ ID NO: 270 - protein, 17 aa, synthetic construct
ATGPRGFGSRKSTSYDY

SEQ ID NO: 271 - protein, 8 aa, synthetic construct
GYISSTHF

SEQ ID NO: 272 - protein, 7 aa, synthetic construct
ITGSDLT

SEQ ID NO: 273 - protein, 11 aa, synthetic construct
RLWGLGLGDGY

SEQ ID NO: 274 - protein, 8 aa, synthetic construct
GRTFSSTA

SEQ ID NO: 275 - protein, 8 aa, synthetic construct
IRWSDYRT

SEQ ID NO: 276 - protein, 16 aa, synthetic construct
VAGHGLNSRFAEAYDY

SEQ ID NO: 277 - protein, 8 aa, synthetic construct
GLTFSSYS

SEQ ID NO: 278 - protein, 8 aa, synthetic construct
ISWSAGRT

SEQ ID NO: 279 - protein, 15 aa, synthetic construct
AAERAPSRRVDAYEY

SEQ ID NO: 280 - protein, 8 aa, synthetic construct
GLTFSSYS

SEQ ID NO: 281 - protein, 8 aa, synthetic construct
ISWSAGRT

SEQ ID NO: 282 - protein, 15 aa, synthetic construct
AAERAPSRQVAAYEY

SEQ ID NO: 283 - protein, 8 aa, synthetic construct
GGTFSTYF

SEQ ID NO: 284 - protein, 8 aa, synthetic construct
IGGSGGSR

SEQ ID NO: 285 - protein, 17 aa, synthetic construct
ATGPRGFGSRKSTSYDY

SEQ ID NO: 286 - protein, 8 aa, synthetic construct
GRPFSSYI

SEQ ID NO: 287 - protein, 8 aa, synthetic construct
INWSGDHT

SEQ ID NO: 288 - protein, 23 aa, synthetic construct
AAKLSAGSSTDTVLHNNRWSWDY

SEQ ID NO: 289 - protein, 8 aa, synthetic construct
GLPFSTYF

SEQ ID NO: 290 - protein, 8 aa, synthetic construct
IGGNGGSR

SEQ ID NO: 291 - protein, 17 aa, synthetic construct
ATGPAAFGSRKTSSYDY

SEQ ID NO: 292 - protein, 8 aa, synthetic construct
ERTAFTYS

SEQ ID NO: 293 - protein, 8 aa, synthetic construct
ISGRDGRT

SEQ ID NO: 294 - protein, 15 aa, synthetic construct
ATSPLVSTDQPDFYS

SEQ ID NO: 295 - protein, 8 aa, synthetic construct
GLTFSSYT

SEQ ID NO: 296 - protein, 8 aa, synthetic construct
ASWSGGNT

SEQ ID NO: 297 - protein, 15 aa, synthetic construct
AAEKAPSRTVAAYEY

SEQ ID NO: 298 - protein, 8 aa, synthetic construct
GRSFITYF

SEQ ID NO: 299 - protein, 8 aa, synthetic construct
IGGNGGSR

SEQ ID NO: 300 - protein, 17 aa, synthetic construct
ATGPRGFGSRKSTSYDY

SEQ ID NO: 301 - protein, 8 aa, synthetic construct
GRSFITYF

SEQ ID NO: 302 - protein, 8 aa, synthetic construct
IGGNGGSR

SEQ ID NO: 303 - protein, 17 aa, synthetic construct
ATGPRGFGSRKSNSYDY

SEQ ID NO: 304 - protein, 8 aa, synthetic construct
GRSFITYF

SEQ ID NO: 305 - protein, 8 aa, synthetic construct
IGGNGGSR

SEQ ID NO: 306 - protein, 17 aa, synthetic construct
ATGPRGFGSRKSTSYDY

SEQ ID NO: 307 - protein, 8 aa, synthetic construct
GRSFITYF

SEQ ID NO: 308 - protein, 8 aa, synthetic construct
IGGNGGSR

SEQ ID NO: 309 - protein, 17 aa, synthetic construct
ATGPRGFGSRKSTSYDY

SEQ ID NO: 310 - protein, 8 aa, synthetic construct
GSITSFNA

SEQ ID NO: 311 - protein, 7 aa, synthetic construct
ITGSGST

SEQ ID NO: 312 - protein, 20 aa, synthetic construct
YADLSTYNAEWNGGAYRNNY

SEQ ID NO: 313 - protein, 8 aa, synthetic construct
GSSSSINA

SEQ ID NO: 314 - protein, 9 aa, synthetic construct
ISRARGDST

SEQ ID NO: 315 - protein, 13 aa, synthetic construct
YVAGNRDLDFRSY

SEQ ID NO: 316 - protein, 8 aa, synthetic construct
GLTFSSYS

SEQ ID NO: 317 - protein, 8 aa, synthetic construct
ISWSGGRT

SEQ ID NO: 318 - protein, 15 aa, synthetic construct
AAERAPSRQVAAYEY

SEQ ID NO: 319 - protein, 8 aa, synthetic construct
GPTFGTYA

SEQ ID NO: 320 - protein, 8 aa, synthetic construct
MSWSNGRT

SEQ ID NO: 321 - protein, 15 aa, synthetic construct
AAEKAPSRKVSAYEY

SEQ ID NO: 322 - protein, 8 aa, synthetic construct
GRTFSSTA

SEQ ID NO: 323 - protein, 8 aa, synthetic construct
IRWSDYRT

SEQ ID NO: 324 - protein, 16 aa, synthetic construct
VAGHRLNSRFAEAYDY

SEQ ID NO: 325 - protein, 8 aa, synthetic construct
GRSFISYF

SEQ ID NO: 326 - protein, 8 aa, synthetic construct
IGGNGGSR

SEQ ID NO: 327 - protein, 17 aa, synthetic construct
ATGPRGFSSRKSTSYDY

SEQ ID NO: 328 - protein, 8 aa, synthetic construct
GRTFSTWH

SEQ ID NO: 329 - protein, 8 aa, synthetic construct
IGGSGGSR

SEQ ID NO: 330 - protein, 17 aa, synthetic construct
ATGPAAFGSRKSTSYDY

SEQ ID NO: 331 - protein, 8 aa, synthetic construct
ERTFDTYT

SEQ ID NO: 332 - protein, 8 aa, synthetic construct
IGGSGGSR

SEQ ID NO: 333 - protein, 17 aa, synthetic construct
ATGPAGFGSRKTTSYDY

SEQ ID NO: 334 - protein, 8 aa, synthetic construct
GRTSSGYA

SEQ ID NO: 335 - protein, 8 aa, synthetic construct
IAWSAGST

SEQ ID NO: 336 - protein, 16 aa, synthetic construct
AAGTRMPSRMTNAYDY

SEQ ID NO: 337 - protein, 8 aa, synthetic construct
GRTFSSTA

SEQ ID NO: 338 - protein, 8 aa, synthetic construct
IRWSDYRT

SEQ ID NO: 339 - protein, 16 aa, synthetic construct
VAGHRLNSRFAEAYDY

SEQ ID NO: 340 - protein, 8 aa, synthetic construct
GRTVSTYF

SEQ ID NO: 341 - protein, 8 aa, synthetic construct
IGGSGGSR

SEQ ID NO: 342 - protein, 17 aa, synthetic construct
ATGPAGFGSRKTTSYDY

SEQ ID NO: 343 - protein, 8 aa, synthetic construct
GRTFSSTA

SEQ ID NO: 344 - protein, 8 aa, synthetic construct
IRWSDYRT

SEQ ID NO: 345 - protein, 16 aa, synthetic construct
VAGHRLNSRFAEAYDY

SEQ ID NO: 346 - protein, 8 aa, synthetic construct
GRTFSSTA

SEQ ID NO: 347 - protein, 8 aa, synthetic construct
IRWSDYRT

SEQ ID NO: 348 - protein, 16 aa, synthetic construct
VAGHRLITRITEAYDY

SEQ ID NO: 349 - protein, 8 aa, synthetic construct
GRLISADS

SEQ ID NO: 350 - protein, 7 aa, synthetic construct
ITSGGGT

SEQ ID NO: 351 - protein, 7 aa, synthetic construct
NCPRYGD

SEQ ID NO: 352 - protein, 8 aa, synthetic construct
GLTFSSYT

SEQ ID NO: 353 - protein, 8 aa, synthetic construct
ASWSGGST

SEQ ID NO: 354 - protein, 15 aa, synthetic construct
AAEKAPSQTVAAYEY

SEQ ID NO: 355 - protein, 8 aa, synthetic construct
GRLISANS

SEQ ID NO: 356 - protein, 7 aa, synthetic construct
ITSGGGT

SEQ ID NO: 357 - protein, 7 aa, synthetic construct
NWPRYGD

SEQ ID NO: 358 - protein, 8 aa, synthetic construct
GRAFRINS

SEQ ID NO: 359 - protein, 8 aa, synthetic construct
ISWSGRDT

SEQ ID NO: 360 - protein, 18 aa, synthetic construct
AARVFFDSGSYAASEYSN

SEQ ID NO: 361 - protein, 8 aa, synthetic construct
GRTTSTYF

SEQ ID NO: 362 - protein, 8 aa, synthetic construct
IGGSGGSR

SEQ ID NO: 363 - protein, 17 aa, synthetic construct
ATGPRGFGSRRSTSYDY

SEQ ID NO: 364 - protein, 8 aa, synthetic construct
GSSSSINA

SEQ ID NO: 365 - protein, 9 aa, synthetic construct
ISRARGDST

SEQ ID NO: 366 - protein, 13 aa, synthetic construct
YVAGNRDLDFRSY

SEQ ID NO: 367 - protein, 8 aa, synthetic construct
ERTFSTYF

SEQ ID NO: 368 - protein, 8 aa, synthetic construct
IGGNGGSR

SEQ ID NO: 369 - protein, 17 aa, synthetic construct
ATGPRGFGSRKSTSYDY

SEQ ID NO: 370 - protein, 8 aa, synthetic construct
GRTSSTYF

SEQ ID NO: 371 - protein, 8 aa, synthetic construct
IGGSGGSR

SEQ ID NO: 372 - protein, 17 aa, synthetic construct
ATGPRGFGSRKSTSYDY

SEQ ID NO: 373 - protein, 8 aa, synthetic construct
GRPFSSYA

SEQ ID NO: 374 - protein, 8 aa, synthetic construct
IGGDGSTT

SEQ ID NO: 375 - protein, 17 aa, synthetic construct
ALDFSLNRIVFGTRADY

SEQ ID NO: 376 - protein, 8 aa, synthetic construct
ATISSTDF

SEQ ID NO: 377 - protein, 7 aa, synthetic construct
ITSSDLT

SEQ ID NO: 378 - protein, 9 aa, synthetic construct
RVWGLGYYY

SEQ ID NO: 379 - protein, 8 aa, synthetic construct
GRTFSMYN

SEQ ID NO: 380 - protein, 8 aa, synthetic construct
IGGNGGSR

SEQ ID NO: 381 - protein, 17 aa, synthetic construct
ATGPRGFGSQKSNSYDY

SEQ ID NO: 382 - protein, 8 aa, synthetic construct
GRTVSTYF

SEQ ID NO: 383 - protein, 8 aa, synthetic construct
IGGSGGSR

SEQ ID NO: 384 - protein, 17 aa, synthetic construct
ATGPAGFGSRKTTSYDY

SEQ ID NO: 385 - protein, 5 aa, synthetic construct
FSSSY

SEQ ID NO: 386 - protein, 8 aa, synthetic construct
IGGNGGSR

SEQ ID NO: 387 - protein, 17 aa, synthetic construct
ATGPRGFGSRKSTSYDY

SEQ ID NO: 388 - protein, 8 aa, synthetic construct
GRAFSTYF

SEQ ID NO: 389 - protein, 8 aa, synthetic construct
IGGSGGSR

SEQ ID NO: 390 - protein, 17 aa, synthetic construct
ATGPRGFGSRKTTSYDY

SEQ ID NO: 391 - protein, 8 aa, synthetic construct
GRTFSSYA

SEQ ID NO: 392 - protein, 8 aa, synthetic construct
ISWTGGRT

SEQ ID NO: 393 - protein, 16 aa, synthetic construct
TARLLGGGWSGEEYDY

SEQ ID NO: 394 - protein, 8 aa, synthetic construct
GRSFITYF

SEQ ID NO: 395 - protein, 8 aa, synthetic construct
IGGNGGSR

SEQ ID NO: 396 - protein, 17 aa, synthetic construct
ATGPRGFGSRKSTSYDY

SEQ ID NO: 397 - protein, 8 aa, synthetic construct
GRTASSYA

SEQ ID NO: 398 - protein, 8 aa, synthetic construct
MPWTGGRT

SEQ ID NO: 399 - protein, 16 aa, synthetic construct
AARLLGGGWSGEEYDY

SEQ ID NO: 400 - protein, 8 aa, synthetic construct
GRSFSTYF

SEQ ID NO: 401 - protein, 8 aa, synthetic construct
IGGNGGSR

SEQ ID NO: 402 - protein, 17 aa, synthetic construct
ATGPRGFGSRKSTSYDY

SEQ ID NO: 403 - protein, 8 aa, synthetic construct
GLTFSSYS

SEQ ID NO: 404 - protein, 8 aa, synthetic construct
ISWSGGRT

SEQ ID NO: 405 - protein, 15 aa, synthetic construct
AAERAPSRQVAAYEF

SEQ ID NO: 406 - protein, 8 aa, synthetic construct
GSISSTHF

SEQ ID NO: 407 - protein, 7 aa, synthetic construct
ITESSLT

SEQ ID NO: 408 - protein, 9 aa, synthetic construct
GVWGLGYAY

SEQ ID NO: 409 - protein, 8 aa, synthetic construct
GSIASTHF

SEQ ID NO: 410 - protein, 7 aa, synthetic construct
LTESSLT

SEQ ID NO: 411 - protein, 9 aa, synthetic construct
GVWGLGFAY

SEQ ID NO: 412 - protein, 8 aa, synthetic construct
GSIFSTNI

SEQ ID NO: 413 - protein, 7 aa, synthetic construct
ITGSDLT

SEQ ID NO: 414 - protein, 9 aa, synthetic construct
RVWGLGYYY

SEQ ID NO: 415 - protein, 8 aa, synthetic construct
GGTFSTYF

SEQ ID NO: 416 - protein, 8 aa, synthetic construct
IGGSGGSR

SEQ ID NO: 417 - protein, 17 aa, synthetic construct
ATGPRGFGSRKSTSYDY

SEQ ID NO: 418 - protein, 8 aa, synthetic construct
GSISSTHF

SEQ ID NO: 419 - protein, 7 aa, synthetic construct
ITTSSAT

SEQ ID NO: 420 - protein, 9 aa, synthetic construct
GVWGVGYAY

SEQ ID NO: 421 - protein, 8 aa, synthetic construct
GYISSTHF

SEQ ID NO: 422 - protein, 7 aa, synthetic construct
ITGSDLT

SEQ ID NO: 423 - protein, 11 aa, synthetic construct
RLWGLGLGYGY

SEQ ID NO: 424 - protein, 8 aa, synthetic construct
GRPFSSYA

SEQ ID NO: 425 - protein, 8 aa, synthetic construct
IGGDGSIT

SEQ ID NO: 426 - protein, 17 aa, synthetic construct
ALDFSFNRIVLGTRADY

SEQ ID NO: 427 - protein, 8 aa, synthetic construct
ERTFSTYF

SEQ ID NO: 428 - protein, 8 aa, synthetic construct
IGGNGGSR

SEQ ID NO: 429 - protein, 17 aa, synthetic construct
ATGPRGFGSRKSTSYDY

SEQ ID NO: 430 - protein, 8 aa, synthetic construct
GRTFSNYD

SEQ ID NO: 431 - protein, 8 aa, synthetic construct
CGWKAEDT

SEQ ID NO: 432 - protein, 19 aa, synthetic construct
AAGLTVNGRLLTRTYEYDI

SEQ ID NO: 433 - protein, 8 aa, synthetic construct
GRTVSTYF

SEQ ID NO: 434 - protein, 8 aa, synthetic construct
IGGSGGSR

SEQ ID NO: 435 - protein, 17 aa, synthetic construct
ATGPAGFGSRKSTSYDY

SEQ ID NO: 436 - protein, 8 aa, synthetic construct
GRSFSTYF

SEQ ID NO: 437 - protein, 8 aa, synthetic construct
IGGNGGSR

SEQ ID NO: 438 - protein, 17 aa, synthetic construct
ATGPRGFGSRKSNSYDY

SEQ ID NO: 439 - protein, 8 aa, synthetic construct
GRSFSTYF

SEQ ID NO: 440 - protein, 8 aa, synthetic construct
IGGNGGSR

SEQ ID NO: 441 - protein, 17 aa, synthetic construct
ATGPRGFGSRKSTSYDY

SEQ ID NO: 442 - protein, 8 aa, synthetic construct
GGTYTTYT

SEQ ID NO: 443 - protein, 8 aa, synthetic construct
IRRTGGDP

SEQ ID NO: 444 - protein, 15 aa, synthetic construct
AGSPLWTSSQDDYRH

SEQ ID NO: 445 - protein, 8 aa, synthetic construct
ERTFSTYF

SEQ ID NO: 446 - protein, 8 aa, synthetic construct
IGGNGGSR

SEQ ID NO: 447 - protein, 17 aa, synthetic construct
ATGPRGFGSRKSTSYDY

SEQ ID NO: 448 - protein, 8 aa, synthetic construct
GSIPSTHF

SEQ ID NO: 449 - protein, 7 aa, synthetic construct
ITGSDVT

SEQ ID NO: 450 - protein, 9 aa, synthetic construct
RVWGVGYDY

SEQ ID NO: 451 - protein, 8 aa, synthetic construct
GSIPSTHF

SEQ ID NO: 452 - protein, 7 aa, synthetic construct
ITGSDLT

SEQ ID NO: 453 - protein, 9 aa, synthetic construct
RVWGVGYDY

SEQ ID NO: 454 - protein, 126 aa, synthetic construct
QVQLQESGGGLVQAGGSLRLSCAASGLTFRNYDLGWFRQAPGKEREFVAGASWSEEDTYYLNSVKGRFTISRDNAKNTVYLQMNSLRPEDTAIYYCAAGLTVNGRLLTRTYEYDHWGQGTQVTVSS

SEQ ID NO: 455 - protein, 120 aa, synthetic construct
QVQLQESGGGLVQPGGSLRLSCVASGSISSIDHVGWYRQAPGKERVMVAFINSGGRTTYPDAVKGRFTISRDGASNTVFLQMDGLKPDDTAVYYCNVLLRDRSGSGRTYWGQGTQVTVSS

SEQ ID NO: 456 - protein, 122 aa, synthetic construct
QVQLQESGGGLVQPGGSLRLSCAASERTAITYSMGWFRQAPGKDRDFVALISGRDGRTAYADSVKGRFTISQNYAANTVWLQMNSLNPEDTAVYYCATSPLVSTDQPDFYSWGQGTQVTVSS

SEQ ID NO: 457 - protein, 111 aa, synthetic construct
QVQLQESGGGLVQAGGSLRLSCAASGSIFRSVPVSWYRQAPEKQREFVARIFVDGSTHLADPVKGRFTISRDNAKKTVYLQMNSLKPEDTAVYYCFMNGDWGQGTQVTVSS

SEQ ID NO: 458 - protein, 113 aa, synthetic construct
QVQLQESGGGLVQAGGSLRLSCAATESRFSIKPMGWYRQAPGKQREFVATITSPGMANYEDSVKGRFTISKDIPKNTVYLQMNSLKPEDTAVYYCRDILSGSWGQGTQVTVSS

SEQ ID NO: 459 - protein, 113 aa, synthetic construct
QVQLQESGGGLVQSGGSLRLSCVASGRLLSDNSMTWYRQAPEKQREFVAHITSGGGTNYADSVKGRFTISRDNAKNTVYSQMISLKPEDTAVYYCNWPRYGDRGQGTQVTVSS

SEQ ID NO: 460 - protein, 124 aa, synthetic construct
QVQLQESGGGLVQAGDSVRLSCAASGRPFSSYAMGWIRQAPGKEREFVAAIGGDGSTTRYTESAKGRFTISRDNAKNTMYLQMNSLIPEDTAVYYCALDFSLNRIVFGTRADYWGQGTQVTVSS

SEQ ID NO: 461 - protein, 124 aa, synthetic construct
QVQLQESGGGLVQGGGSLRLSCAASGRTFSTWHAAWFRQAPGKEREFVATIGGSGGSRYYADPVEGRFTISRDNAKNTVYLQMTALKVEDTAVYYCATGPAAFGSRKGTSYDYWGQGTQVTVSS

SEQ ID NO: 462 - protein, 125 aa, synthetic construct
QVQLQESGGGLVQAGGSLRLSCAASGRAFRINSIGWFRQAPGKEREFVAAISWSGRDTYYDDSVKGRFTISRDNAKNTVYLEMNSLKPADTAVYSCAARVFFDSGSYAASEYSNWGQGTQVTVSS

SEQ ID NO: 463 - protein, 123 aa, synthetic construct
QVQLQESGGGLVQAGGSLRVSCGASGDTFSNYAMAWFRQLAGKEREFVAAISWHGGRATYADSVQGRFTISRDNAKNTVYLQMNSLKPEDTAVYVCAARLLGGGWSGEEYDYWGQGTQVTVSS

SEQ ID NO: 464 - protein, 123 aa, synthetic construct
QVQLQESGGGLVQAGGSLRLSCAFSGRTFSGTAMGWFRQPPGKEREFVASIWWSRYATDYADSVKDRFTVSRDNAANTVYLQMNSLKPEDTAVYYCAAGHRGYSRFAEAYDYWGQGTQVTVSS

SEQ ID NO: 465 - protein, 126 aa, synthetic construct
QVQLQESGGGLVQCGGSLRLSCAASERTFGIRTIGWFRQAPGKEREFVGAIKWAGGNTHYADPVKGRFTISRDNAKNTGYLQMNSLKPEDTAVYVCAAAKVYYYTPTMGPGSYEFWGQGTQVTVSS

SEQ ID NO: 466 - protein, 124 aa, synthetic construct
QVQLQESGGGLVQAGGSLRLSCVVSGRTGSHLDMAWFRQAPGKEREFVATISRDGFRIFYADSVKGRFTMSRDNGKNSVYLQMNSLKPEDTAVYYCAADAAGFGSRFVSSYDYWGQGTQVTVSS

SEQ ID NO: 467 - protein, 126 aa, synthetic construct
QVQLQESGGGLVQAGGSLRLSCAASGRTFDSDRFGMGWFRQAPGKEREFVAQINWRGGGAFYADSVKGRFTISRDTVKNTVTLQMNSLQVEDTAVYFCVADIAAWGARSAASYEYWGRGTQVTVSS

SEQ ID NO: 468 - protein, 117 aa, synthetic construct
QVQLQESGGGLVQPGGSLRLSCVASRAISSRWPMGWYRQAPGKQRELVAQISHGSITNIMDSVKGRFTISRDYAESTVYLQMNSLKPEDTAVYYCYAEDWDTRVQYWGQGTQVTVSS

SEQ ID NO: 469 - protein, 122 aa, synthetic construct
QVQLQESGGGLVQAGDSLRLSCAASGLTFSSYTMAWFRQAPGKEREFVAAASWSGGSTYYADSVEGRFTISRDYAKNTVYLEMNSLKPEDTAVYYCAAEKAPSRTVAAYEYWGQGTQVTVSS

SEQ ID NO: 470 - protein, 123 aa, synthetic construct
QVQLQESGGGLVQAGGSLRLSCAASGRTSSGYAMGWFRQAPGKEREFVAAIAWSAGSTYYADSVQGRFTISRDKPKITVYLQMDSLTPEDTAVYYCAAGTRMPSRMTNAYDYWGRGTQVTVSS

SEQ ID NO: 471 - protein, 123 aa, synthetic construct
QVQLQESGGGLVQAGGSLRLSCAASGRTSSGYAMGWFRRAPGKEREFVAAISWSGGSTYYADSLRGRFTISRDKPKMTVYLQMDSLTPEDTAVYYCAAGTRQVSQTTQAYDYWGRGTQVTVSS

SEQ ID NO: 472 - protein, 130 aa, synthetic construct
QVQLQESGGGLAQPGGSLRLSCAASGRPFSSYILGWFRQAPGKERELVAQINWSGDHTYYANSVKGRFTISRDNAENTGYLQMNSLKPEDTAVYFCAAKLSAGSSTDTVLHNNRWSWDSWGQGTQVTVSS

SEQ ID NO: 473 - protein, 122 aa, synthetic construct
QVQLQESGGGLVQTGGSLRLSCAASGGTYTTWTMGWFRQAPGKDRTIVAAIRRTGGDPYYTTSVEGRFTISRDNAKNTVYLQMNSLKPEDTAVYYCAASPLWTSSQDDYRHWGQGTQVTVSS

SEQ ID NO: 474 - protein, 110 aa, synthetic construct
QVQLQESGGGLVQAGGSLRLSCSASGAYLIVSDMNWYRQSPGKERELVATIGRGGTNYAESVKGRFTISRDNTKNFWYLQMSSLKPEDTAIYYCNIVDYWGQGTQVTVSS

SEQ ID NO: 475 - protein, 125 aa, synthetic construct
QVQLQESGGGLVQPGGSLRLSCVASGFTFSSYWMYWVRQRPGKGLEWVSGMSPEGDMTGYTDSVKGRFTISRDNAKNTLYLQMNSLKSDDTAVYSCVKGRTHGSGLYGARDFESRGQGTQVTVSS

SEQ ID NO: 476 - protein, 117 aa, synthetic construct
QVQLQESGGGLVQAGGSLRLSCAASGYISSTHFMGWYRQAPGKQRELVAGITGSDLTNYADSVKGRFIISRDNAKNTVALEMNSLKPEDTAVYHCRLWGLGLGDGYWGQGTQVTVSS

SEQ ID NO: 477 - protein, 123 aa, synthetic construct
QVQLQESGGGVVQSGGSLRLSCAASGFTLSDYWMSWLRQAPGKGLEWVSVIKAGDDTTYDLGSVKGRFTISRDNAKNTLYLQMNSLESEDTAVYYCARSPYGTYRLDRRYDFRGQGTQVTVSS

SEQ ID NO: 478 - protein, 121 aa, synthetic construct
QVQLQESGGGLVQAGGSLTLSCAASGSSSSINAMAWYRQAPGKQRELVAHISRARGDSTIYADSVKGRFTISRENAKNTVYLQMNSLKPEDTAVYYCYVAGDRSLDFRSYWGQGTQVTVSS

SEQ ID NO: 479 - protein, 115 aa, synthetic construct
QVQLQESGGGLVQAGGSLRLSCAASGTISSTDFMGWYRQAPGKQRELVAGITGSDLTNYADSVKGRFIISRDNAPNTVALQMNSLKPEDTATYYCRVWGLGYYYWGQGTQVTVSS

SEQ ID NO: 480 - protein, 124 aa, synthetic construct
QVQLQESGGGLVQPGGSLRLSCAASGRTFDYHAVAWFRQAPGKERELVATIGGSGGSRYYADPVLGRFTISRDNAKNTVYLQMNALKPEDTPVSYCATGPAGYGSRKSTSYDYWGQGTQVTVSS

SEQ ID NO: 481 - protein, 113 aa, synthetic construct
QVQLQESGGGVVQAGGSLRLSCASSEEWFRINNMGWYRQAPGKQRELVAYITPSGSTNYADFVKGRFTISRDNAKKTVLLQMDSLRPEDTAIYYCRDISGGSWGQGTQVTVSS

SEQ ID NO: 482 - protein, 128 aa, synthetic construct
QVQLQESGGGLVQAGESLRISCAASGRTLTDRTLTDYAVGWFRQPPGKEREFVAAIRWSDYRTDYADSVKDRFTVSRDNAKNTVYLQMNSLKPEDTAVYYCVAGHRLNSRFAEAYNYWGQGTQVTVSS

SEQ ID NO: 483 - protein, 124 aa, synthetic construct
QVQLQESGGGLVQAGGSLRLSCAASERPFSSYAMGWFRQAPGKEREFVAAIGGDGSITQYTESAAGRFTISRDNAKNTMYLQMNSLKPEDTATYYCALDFSFNRIVLGSRADYWGQGTQVTVSS

SEQ ID NO: 484 - protein, 121 aa, synthetic construct
QVQLQESGGGLVQAGGSLRLSCAASGRTFNAMAWFRQPPGKEREFIAAIRWNGYMTDYADSVKGRFTVSRDNAKNTEFLQMNSLKPEDTAVYYCAAGDRGSSRFVAAYDYWGQGTQVTVSS

SEQ ID NO: 485 - protein, 123 aa, synthetic construct
QVQLQESGGGLVQTGGSLRLSCAASGFTFDDYAIGWFRQAPGKDREGVSCISSSRATTSYADAVKGRFRTSIDNAKKTVYLQTNNLEPEDTAVYYCAAGTRMPSRMTNAYDYWGRGTQVTVSS

SEQ ID NO: 486 - protein, 119 aa, synthetic construct
QVQLQESGGGLVQPGGSLRLSCVASRAISSRWPMGWYRQAPGKQRELVAQISHGSITNIMDSVKGRFTISRDYAESTVYLQMNSLKPEDTAVYYCYAAGQSTAPSASYWGQGTQVTVSS

SEQ ID NO: 487 - protein, 122 aa, synthetic construct
QVQLQESGGGLVQPGGSLRLSCAASVRTLTAMGWFRQAPGKERELVGSMRWSDGSTYYTDSVKGRFTISRDNAKNTVYLQMNSLKPEDTAVYYCAADQVIFYSRKPTDYDYWGQGTQVTVSS

SEQ ID NO: 488 - protein, 126 aa, synthetic construct
QVQLQESGGGLVQAGGSLRLSCAASGSISSFNAMGWYRQAPGQQRQLVARITGSGSTNYADSVKGRFTISRVNAKNTVVLQMNSLRSDDTSVYLCYADLSTYNAAWNGGVYRNNYWGQGTQVTVSS

SEQ ID NO: 489 - protein, 124 aa, synthetic construct
QVQLQESGGGLVQAGGSLRLSCAASGLPFSTYFMAWFRQAPGEEREFVASIGGNGGSRYYADPVEGRFTISRDNAKTTVYLQMNALKPEDTAVYYCATGPAAFGSRKTSSYDYWGQGTQVTVSS

SEQ ID NO: 490 - protein, 113 aa, synthetic construct
QVQLQESGGGLVQAGGSLRLSCAATESRFSSKPMGWYRQAPGKQREYVATITSPGMANYADSVRGRFTISKDIPKNTVYLQMDSLKPEDTAVYYCRDILSDSWGQGTQVTVSS

SEQ ID NO: 491 - protein, 124 aa, synthetic construct
QVQLQESGGGSVQAGGSLRLSCAASGRSFSTYFAAWFRQAPGKEREFVATIGGNGGSRYYADPVQGRFTISRDNAKNTVYLQMNALKPEDTAVYYCATGPRGFGSRKSTSYDYWGQGTQVTVSS

SEQ ID NO: 492 - protein, 123 aa, synthetic construct
QVQLQESGGGLVQPGGSLRLSCAASGFTLSDYWMSWLRQAPGKGLEWVSVIASGGSDTYYLNSVKGRFTISRDNDKNTLYLQMNNLKSEDTAVYYCARSPYGTYRLDRRYDTRGQGTQVTVSS

SEQ ID NO: 493 - protein, 117 aa, synthetic construct
QVQLQESGGGLVQAGGSLRLSCAASGYISSTHFMGWYRQAPGKQRELVAGITGSDLTNYADSVKGRFIISRDNAKNTVALEMNSLKPEDTAVYYCRLWGLGLGDGYWGQGTQVTVSS

SEQ ID NO: 494 - protein, 115 aa, synthetic construct
QVQLQESGGGLVQAGGSLGLSCAASGSIPSTHFMGWYRQPPGKQRELVAGITGSDLTNYADSVKGRFIISREHAQNTVALQMNSLKPEDTAVYYCRVWGVGYDYWGQGTQVTVSS

SEQ ID NO: 495 - protein, 124 aa, synthetic construct
QVQLQESGGGLVQAGDSVRLSCAASGRPFSSYAMGWIRQAPGKEREFVAAIGGDGSTTRYTESAKGRFTISRDNAKNTMYLRMNSLIPEDTAVYYCALDFSLNRIVFGTRADYWGQGTQVTVSS

SEQ ID NO: 496 - protein, 113 aa, synthetic construct
QVQLQESGGGLVQSGGSLRLSCVVSGRLISADSITWYRQAPEKQREFVAHITSGGGTNYADFVKGRFAISRDNAKNTVYLQMNSLKPDDTAVYYCHWPRYGDWGQGTQVTVSS

SEQ ID NO: 497 - protein, 122 aa, synthetic construct
QVQLQESGGGLVQAGGSLRLSCAASGLTFSSYSMGWFRQAPGKEREFVGAISWSNGRTYYADFVKGRFTISRDNAKNTVYMQMNSLKPEDTAVYYCAAEKAPSRTVAAYEYWGQGTQVTVSS

SEQ ID NO: 498 - protein, 121 aa, synthetic construct
QVQLQESGGGLLQAGESLRLSCAASGSSSSINAMAWSRQAPGKQREMVAHISRARGDSTIYADSVKGRFTISRENAKNTVYLQMNNLKPEDTAVYFCYVAGNRDLDFRSYWGQGTQVTVSS

SEQ ID NO: 499 - protein, 115 aa, synthetic construct
QVQLQESGGGLVQAGGSLRLSCAASGSISSTDFMGWYRQAPGKQRELVAGITGSDLTNYADSVKGRFIISREHAGNTMALQMNSLKPEDTAVYYCRVWGLGYAYWGQGTQVTVSS

SEQ ID NO: 500 - protein, 122 aa, synthetic construct
QVQLQESGGGLVQAGGSLRLSCAASGLTFSSYSMGWFRQAPGKEREFVGAISWSGGRTYYADSVTGRFTISRDNAKNTVYLQMNNLKPEDTGVYYCAAERAPSRQVAAYEFWGQGTQVTVSS

SEQ ID NO: 501 - protein, 123 aa, synthetic construct
QVQLQESGGGLVQAGGSLRLSCGASGRTVSNYAMGWFRQAAGKEREFVAAVAWTGGRTTYADSVKGRFTLSRNSAKDTVYLQMNSLKPEDTAVYYCAARLLGGGWSGEEYDSWGQGTQVTVSS

SEQ ID NO: 502 - protein, 117 aa, synthetic construct
QVQLQESGGGLVQPGGSLRLSCVTSRSISSRWPMGWYRQAPGKQRELVAQISHGSIINIMDSVKGRFTISRDYAESTVYLQMNSLKPEDTAVYTCYAEDWDTRVQYWGQGTQVTVSS

SEQ ID NO: 503 - protein, 115 aa, synthetic construct
QVQLQDSGGGLVQPGGSLGLSCAASGSIPSTHFMGWYRQPPGKQRELVAGITGSDLTNYADSVKGRFIISREHAQNTVALQMNSLKPEDTAVYYCRVWGVGYDYWGQGTQVTVSS

SEQ ID NO: 504 - protein, 124 aa, synthetic construct
QVQLQESGGGLVQAGGSLRLSCLASERTFSTYFKAWFRQAPGKEREFVATIGGNGGSRYYAEPVEGRFFISRDNAKNTVYLEMNALKPEDTAVYYCATGPRGFGSRKSTSYDYWGQGTQVTVSS

SEQ ID NO: 505 - protein, 124 aa, synthetic construct
QVQLQESGGGLVQAGGSLRLSCAASGRIFSTYSMGWFRQAPGKEREFVATIGGSGGSRYYADPVEGRFTISRDNAKNTMYLQMNALKPEDTAVYYCATGPRGFGSRKTTSYDYWGQGTQVTVSS

SEQ ID NO: 506 - protein, 123 aa, synthetic construct
QVQLQESGGGLVQAGNSLRLSCVASGRTVSSYAMGWFRQALGKEREFVAAMSWTGGRTTYADSVKGRFTMSRDNAKSTAYLQMNNLKPEDTAVYYCAGRLLGGGWSGEEYGYWGQGTQVTVSS

SEQ ID NO: 507 - protein, 124 aa, synthetic construct
QVQLQESGGGLVQDGGSLRLSCAASGRAFSTYFMAWFRQGPGKEREFVATIGGNGGSRYYADPVEGRFTISRDNAKNTMYLQMNALKPEDTDVYYCETGPRGFGSRKNISYDYWGQGTQVTVSS

SEQ ID NO: 508 - protein, 122 aa, synthetic construct
QVQLQESGGGLLQAEGSLRLSCAASGLTFSSYSMGWFRQAPGKEREFVGAISWSGGRTYYADSVQGRFTISRDNSKNTVYLQMNSLKPEDTAVYYCAAERAPSRQVAAYEYWGQGTQVTVSS

SEQ ID NO: 509 - protein, 122 aa, synthetic construct
QVQLQESGGGLVQAGGSLRLSCAASGFTFSTYSMGWFRQAAGKEREFVGAISWSNGRTYYADSVEGRFTISRDNAKNTVYLQMNSLKPEDTAVYYCAAEKAPSRKVSAYEYWGQGTQVTVSS

SEQ ID NO: 510 - protein, 124 aa, synthetic construct
QVQLQESGGGLVQAGGSVRLSCLASERTFSTYFKAWFRQAPGKEREFVATIGGNGGSRYYAEPVEGRFFISRDNAKNTVYLEMNALKPEDTAVYYCATGPRGFGSRKSTSYDYWGQGTQVTVSS

SEQ ID NO: 511 - protein, 122 aa, synthetic construct
QVQLQESGGGLVPAGGSLRLSCAASGLTFSSYSMGWFRQAPGKEREFVGAISWSGGRTYYADSVTGRFTISRDNAKNTVYLQMNNLKPEDTGVYYCAAERAPSRQVAAYEFWGQGTQVTVSS

SEQ ID NO: 512 - protein, 115 aa, synthetic construct
QVQLQDSGGGLVQAGGSLGISCAAYGSIPSTHFMGWYRQPPGKQRELVAGITGSDLTNYADSVKGRFIISREHAQNTVALQMNSLKPEDTAVYYCRVWGVGYDYWGQGTQVTVSS

SEQ ID NO: 513 - protein, 124 aa, synthetic construct
QVQLQESGGGLVQAGGSLRLSCAASGRTTSTYFMAWFRQAPGKEREFVATIGGSGGSRHYADPVQGRFTISRDNAKSTMYLQMNALKPEDTAAYYCATGPRGFGSRKSTSYDYWGQGTQVTVSS

SEQ ID NO: 514 - protein, 115 aa, synthetic construct
QVQLQESGGGLVQAGGSLRLSCAVSGSIPSTHFMGWYRQPPGKQRELVAGITGSDLTNYADSVKGRFIISREHAKNTVALQMNSLKPEDTAVYYCRVWGVGYDYWGQGTQVTVSS

SEQ ID NO: 515 - protein, 123 aa, synthetic construct
QVQLQESGGGLVQAGDSLRVSCGASGGTFSSYAMAWFRQAAGKEREFVAAIAWHGGRTSYADSVRGRFTISRDNAKNTGYLQMNSLKPADTAVYYCAARLLGGGWSGEEYEYWGQGTQVTVSS

SEQ ID NO: 516 - protein, 121 aa, synthetic construct
QVQLQESGGGVVQAGGSLTLSCAASGSSSSINAMAWYRQAPGKQRELVAHISRARGDSTIYADSVKGRFTISRENAKNTVYLQMNSLKPEDTAVYYCYVAGDRSLDFRSYWGQGTQVTVSS

SEQ ID NO: 517 - protein, 124 aa, synthetic construct
QVQLQESGGGLVQAGGSLRLSCAASGRSFSTYFMAWFRQAPGKEREFVATIGGNGGSRYYADPVSGRFTISRDNAKNTVYLQMNALKPEDTAVYYCATGPRGFGSRKSTSYDYWGQGTQVTVSS

SEQ ID NO: 518 - protein, 124 aa, synthetic construct
QVQLQESGGGLVQAGGSLRLSCAASGRTVSTYFMTWFRQAPGKEREFVATIGGSGGSRYYADPVQGRFTISRDNAKNTVYLQMNALKPEDTAVYYCATGPAGFGSRKSTSYDYWGQGTQVTVSS

SEQ ID NO: 519 - protein, 122 aa, synthetic construct
QVQLQESGGGLVQAGDSLRLSCAASGLTFSSYSMGWFRQAPGKEREFVGAISWSGGRTYYADSVQGRFTISRDNSKNTVYLQMNSLKPEDTAVYYCAAERAPSRQVAAYEYWGQGTQVTVSS

SEQ ID NO: 520 - protein, 114 aa, synthetic construct
QVQLQESGGGSVQAGGSLLSCAACGSIASTHFMGWYRQAPGKQRELVAGLTESSLTNYADSVKGRFIISREHAKNTVALQMNSLKPEDTAVYYCGVWGLGFAYWGQGTQVTVSS

SEQ ID NO: 521 - protein, 124 aa, synthetic construct
QVQLQESGGGLVQAGGSLRLSCAYSERTFDTYTMAWFRQAPGKEREFVATIGGSGGSRYYTDPVMGRFTISRDNAKNTVYLQMNALKPEDTAVYYCATGPAGFGSRKTTSYDYWGQGTQVTVSS

SEQ ID NO: 522 - protein, 122 aa, synthetic construct
QVQLQESGGGLVQAGGSLRLSCAASGLTFSSYSMGWFRQAPGKEREFVGAISWSAGRTYYADSVQGRFTISRDNSKNTVYLKMNSLKPEDTAKYYCAAERAPSRQVAAYEYWGQGTQVTVSS

SEQ ID NO: 523 - protein, 122 aa, synthetic construct
QVQLQESGGGLVQAGASLRLSCAASGLTFSSYSMGWFRQAPGKEREFVGAISWSAGRTYYADSVQGRFTISRDNSKNTVYLQMNSLKPEDTAKYYCAAERAPSRQVAAYEYWGQGTQVTVSS

SEQ ID NO: 524 - protein, 117 aa, synthetic construct
QVQLQESGGGFVQPGGSLRLSCVTSRSISSRWPMGWYRQAPGKQRELVAQISHGSIINIMDSVKGRFTISRDYAESTVYLQMNSLKPEDTAVYTCYAEDWDTRVQYWGQGTQVTVSS

SEQ ID NO: 525 - protein, 124 aa, synthetic construct
QVQLQESGGGLVQAGDSVRLSCAASGRPFSSYAMGRIRQAPGKEREFVAAIGGDGSTTRYTESAKGRFTISRANAKNTMYLQMNSLIPEDTAVYSCALDFSLNRIVFGTRADYWGQGTQVTVSS

SEQ ID NO: 526 - protein, 122 aa, synthetic construct
QVQLQESGGGLVQAGGSLRLSCAASGLTFSSYSLGWFRQAPGKEREFVGAISWSGGRTYYADSVTGRFTISRDNAKNTVYLQMNNLKPEDTGVYYCAAERAPSRQVAAYEFWGQGTQVTVSS

SEQ ID NO: 527 - protein, 113 aa, synthetic construct
QVQLQESGGGLVQAGGSLRLSCAATGDRFSIKPMGWYRQAPGKQREFVATITSPGTANYEDSVKGRFTISKDIAKNTVYLQMNSLKPEDTGVYYCRDILSDSWGQGTQVTVSS

SEQ ID NO: 528 - protein, 127 aa, synthetic construct
QVQLQESGGGLVQAGGSLRLSCAASGRTFSNYDMGWFRQAPGKEREFVAACSWGGENTAYYVNSVKGRFTISRDNAKNTVYLQMNSLKPEDTAIYYCVAGLTVNGRLLTRTYEYDNWGQGTQVTVSS

SEQ ID NO: 529 - protein, 123 aa, synthetic construct
QVQLQESGGGLVQAGGSLRLSCAASGRTFSSTAMGWFRQPPGKEREFVAAIRWSDYRTDYADSVKDRFTVSRDNAKNTVYLQMNSLKPEDTAVYYCVAGHRLNSRFAEAYDYWGQGTQVTVSS

SEQ ID NO: 530 - protein, 124 aa, synthetic construct
QVQLQESGGGLVQAGGSLRLSCAASGRAFSTYHVAWFRQAPGKEREFVATIGGSGGSRYYADPVKGRFTISRDNAKNTVYLQMNALKPEDTAVYYCATGPAGFGSRRSTSYDYWGQGTQVTVSS

SEQ ID NO: 531 - protein, 115 aa, synthetic construct
QVQLQESGGGLVQAGGSLRLSCAASGSISSTHFMGWYRQAPGKQRELVAGITESSLTSYATSVKGRFIISREHAKNTVALQMNSLEPEDTAVYYCGVWGLGYAYWGQGTQVTVSS

SEQ ID NO: 532 - protein, 124 aa, synthetic construct
QVQLQESGGGLVQAGGSLRLSCAASGRTTSTYFMAWFRQAPGKEREFVATIGGSGGSRHYADPVQGRFTISRDNAKSTMYLQMNALKPEDTAVYYCATGPRGFGSRKSTSYDYWGQGTQVTVSS

SEQ ID NO: 533 - protein, 113 aa, synthetic construct
QVQLQESGGGLVQAGGSLRLSCAATGSRFSTKPMGWYRQAPGKQRDYVATITTPGMANYAASVKGRFTISKDITKNTVYLQMNSLKPEDTATYYCRDILSDDWGQGTQVTVSS

SEQ ID NO: 534 - protein, 124 aa, synthetic construct
QVQLQESGGGLVQPGGSLRLSCAASGRSFSTYFMAWFRQAPGKEREFVATIGGNGGSRYYADPVEGRFTISRDNAKNTVYLQMNALKPEDTAVYYCATGPRGFGSRKSTSYDYWGQGTQVTVSS

SEQ ID NO: 535 - protein, 123 aa, synthetic construct
QVQLQESGGGLVQAGGSLRLSCAASGRTFSSTAMGWFRQPPGEEREFVAAIRWSDYRTDYADSVKDRFTVSRDNAKNTVYLQMNSLKPEDTAVYYCVAGHRLNSRFAEAYDYWGQGTQVTVSS

SEQ ID NO: 536 - protein, 124 aa, synthetic construct
QVQLQESGGGLVQAGGSLRLSCAASGLPFSTYFMAWFRQAPGKEREFVASIGGNGGSRYYADPVEGRFTISRDNAKTTVYLQMNALKPEDTAVYYCATGPAAFGSRKTSSYDYWGQGTQVTVSS

SEQ ID NO: 537 - protein, 124 aa, synthetic construct
QVQLQESGGGLVQAGESLRLSCAASGRSFSTDFMAWFRQAPGKEREFVATIGGNGGSRYYADPVQGRFTISRDNAKNTVYLQMNALKPEDTAVYYCATGPRGFGSRKSTSYDYWGQGTQVTVSS

SEQ ID NO: 538 - protein, 113 aa, synthetic construct
QVQLQESGGGLVQSGGSLRLSCVASGRLISANSMGWYRQAPEKQREFVAHITSGGGTSYADSVKGRFTISRDNAKNTVYLQMNSLKPEDTAVYYCNWPRYGDWGQGTQVTVSS

SEQ ID NO: 539 - protein, 122 aa, synthetic construct
QVQLQESGGGLVQAGGSLRLSCAASGLTFSSYSMGWFRQAPGKEREFVGAISWSAGRTYYSDSVEGRFTISRDNAKNTVYLQMNSLKAEDTAVYYCAAERAPSRRVDAYEYWGQGTQVTVSS

SEQ ID NO: 540 - protein, 124 aa, synthetic construct
QVQLQESGGGLVEAGGSLRLSCAASGRSFSTYFAAWFRQAPGKEREFVATIGGNGGSRYYADPVQGRFTISRDNAKNTVYLQMNALKPEDTAVYYCATGPRGFGSRKSTSYDYWGQGTQVTVSS

SEQ ID NO: 541 - protein, 122 aa, synthetic construct
QVQLQESGGGLVQPGGSLRLSCAASGLTFSSYSMGWFRQAPGKEREFVGAISWSGGRTYYADSVTGRFTISRDNAKNTVYLQMNNLKPEDTGVYYCAAERAPSRQVAAYEFWGQGTQVTVSS

SEQ ID NO: 542 - protein, 122 aa, synthetic construct
QVQLQESGGGLVQAGGSLRLSCAASGLTFSSYSMGWFRQAPGKEREFVGAISWSYGSTYYADSVKGRFAISRDNAQNTVYLQMNSLKPEDTAVYYCAAEKAPSRRVAAYEYWGQGTQVTVSS

SEQ ID NO: 543 - protein, 124 aa, synthetic construct
QVQLQESGGGLVQAGGSLRLSCAASGRSFSTYFMAWFRQAPGKEREFVATIGGNGGSRYYADPVEGRFTISRDNAKNTVYLQMNALKPEDTAVYYCATGPRGFGSRKSTSYDYRGQGTQVTVSS

SEQ ID NO: 544 - protein, 117 aa, synthetic construct
QVQLQESGGGLVQAGGSLRLSCAASGYISSTHFMGWYRQAPGEQRELVAGITGSDLTNYADSVKGRFIISRDNAKNTVALEMNSLKPEDTAVYYCRLWGLGLGDGYWGQGTQVTVSS

SEQ ID NO: 545 - protein, 123 aa, synthetic construct
QVQLQESGGGLVQAGGSLRLSCAASGRTFSSTAMGWFRQPPGKEREFVAAIRWSDYRTDYADSVKDRFTVSRDNAKNTVYLQMNSLKPEDTAVYYCVAGHGLNSRFAEAYDYWGQGTQVTVSS

SEQ ID NO: 546 - protein, 122 aa, synthetic construct
QVQLQESGGGLVQAGGSLRLSCAARGLTFSSYSMAWFRQAPGKEREFVGAISWSAGRTYYSDSVEGRFTITRDNAKNTVYLQMNSLKPEDTAVYYCAAERAPSRRVDAYEYWGQGTQVTVSS

SEQ ID NO: 547 - protein, 122 aa, synthetic construct
QVQLQESGGGLVQAGGSLRLSCAASGLTFSSYSMGWFRQAPGKEREFVGAISWSAGRTYYADSVQGRFTISRDNSKNTVYLQMNSLKPEDTAKYYCAAERAPSRQVAAYEYWGQGTQVTVSS

SEQ ID NO: 548 - protein, 124 aa, synthetic construct
QVQLQESGGGLVQAGGSLRLSCAASGGTFSTYFMTWFRQAPGKEREFVATIGGSGGSRHYADPVQGRFTISRDNAKSTMYLQMNALKPEDTAVYYCATGPRGFGSRKSTSYDYWGQGTQVTVSS

SEQ ID NO: 549 - protein, 130 aa, synthetic construct
QVQLQESGGGLAQPGGSMRLSCAASGRPFSSYILGWFRQAPGKERELVSQINWSGDHTYYANSVKGRFTISRDNAENTGYLQVNSLKPEDTAVYFCAAKLSAGSSTDTVLHNNRWSWDYWGQGTQVTVSS

SEQ ID NO: 550 - protein, 124 aa, synthetic construct
QVQLQESGGGLVQPGGSLRLSCAASGLPFSTYFMAWFRQAPGKEREFVASIGGNGGSRYYADPVEGRFTISRDNAKTTVCLQMNALKPEDTAVYYCATGPAAFGSRKTSSYDYWGQGTQVTVSS

SEQ ID NO: 551 - protein, 122 aa, synthetic construct
QVQLQESGGGLVQAGGSLRLSCAASERTAFTYSMGWFRQAPGKDRDFVALISGRDGRTAYADSVKGRFTISQNYAANTVWLQMNSLKPEDTAVYYCATSPLVSTDQPDFYSWGQGTQVTVSS

SEQ ID NO: 552 - protein, 122 aa, synthetic construct
QVQLQESGGGLVQAGDSLRLSCAASGLTFSSYTMAWFRQAPGKEREFVAAASWSGGNTYYADSVEGRFTISRDYAKNTVYLEMNSLKPEDTAVYYCAAEKAPSRTVAAYEYWGQGTQVTVSS

SEQ ID NO: 553 - protein, 124 aa, synthetic construct
QVQLQESGGGLVQAGGSLRLSCSASGRSFITYFMAWFRQFPGKEREFVATIGGNGGSRYYADPVQGRFTISRDNAKNTGYLQMNALQPEDTAVYYCATGPRGFGSRKSTSYDYRGQGTQVTVSS

SEQ ID NO: 554 - protein, 124 aa, synthetic construct
QVQLQESGGGLVQAGGSLRLSCAASGRSFITYFMAWFRQAPGREREFVATIGGNGGSRYYADPVEGRFTISRDNAKNTLYLQMNALEPEDTAIYYCATGPRGFGSRKSNSYDYWGQGTQVTVSS

SEQ ID NO: 555 - protein, 124 aa, synthetic construct
QVQLQESGGGLVQAGGSLRLSCAASGRSFITYFMAWFRQTPGKEREFVATIGGNGGSRYYADPVEGRLTISRDNAKNTVYLQMNALKPEDTAVYYCATGPRGFGSRKSTSYDYWGQGTQVTVSS

SEQ ID NO: 556 - protein, 124 aa, synthetic construct
QVQLQESGGGLVQAGGSLTLSCAASGRSFITYFMAWFRQAPGKEREFVATIGGNGGSRYYADPVQGRFTISRDNAKNTMYLQMNALKPEDTAVYYCATGPRGFGSRKSTSYDYWGQGTQVTVSS

SEQ ID NO: 557 - protein, 126 aa, synthetic construct
QVQLQESGGGLVQAGGSLSLSCAASGSITSFNAMGWYRQAPGQQRQLVARITGSGSTNYADSVKGRFTISRVGAKNTVVLQMNSLKSEDTSVYLCYADLSTYNAEWNGGAYRNNYWGQGTQVTVSS

SEQ ID NO: 558 - protein, 121 aa, synthetic construct
QVQLQESGGGLVQAGGSLRLSCAASGSSSSINAMAWYRQAPGKQREMVAHISRARGDSTIYADSVKGRFTISRENAKNTVYLQMNNLKPEDTAAYFCYVAGNRDLDFRSYWGQGTQVTVSS

SEQ ID NO: 559 - protein, 122 aa, synthetic construct
QVQLQESGGGLVQAGGSLRLSCAASGLTFSSYSMGWFRQAPGKEREFVGAISWSGGRTYYADSVQGRFTISRDNSKNTVYLQMNSLKPEDTAVYYCAAERAPSRQVAAYEYWGQGTQVTVSS

SEQ ID NO: 560 - protein, 122 aa, synthetic construct
QVQLQESGGGLVQAGDSLRLSCAASGPTFGTYAMGWFRQAPGKERDFVAAMSWSNGRTYYADSVEGRFTISRDNAKNTVYLQMNSLKPEDTAVYYCAAEKAPSRKVSAYEYWGQGTQVTVSS

SEQ ID NO: 561 - protein, 123 aa, synthetic construct
QVQLQESGGGLVQAGGSLRLSCAASGRTFSSTAMGWFRQPPGKEREFVAAIRWSDYRTDYADSVKDRFTVSRDNAKNTVYLQMNSLEPEDTAVYYCVAGHRLNSRFAEAYDYWGQGTQVTVSS

SEQ ID NO: 562 - protein, 124 aa, synthetic construct
QVQLQESGGGLVQAGGSLRLSCAASGRSFISYFMAWFRQAPGTEREFVATIGGNGGSRYYADPVEGRFTISRDNAKNTLYLQMNALKPEDTAVYYCATGPRGFSSRKSTSYDYWSQGTQVTVSS

SEQ ID NO: 563 - protein, 124 aa, synthetic construct
QVQLQESGGGLVQGGGSLRLSCAASGRTFSTWHAAWFRQAPGKEREFVATIGGSGGSRYYADPVEGRFTISRDNAKNTVYLQMTALKVEDTAVYYCATGPAAFGSRKSTSYDYWGQGTQVTVSS

SEQ ID NO: 564 - protein, 124 aa, synthetic construct
QVQLQESGGGLVQAGGSLRLSCAYSERTFDTYTMAWFRQAPGKEREFVATIGGSGGSRYYTDPVMSRFTISRDNAKNTVYLQMNALKPEDTAVYYCATGPAGFGSRKTTSYDYWGQGTQVTVSS

SEQ ID NO: 565 - protein, 123 aa, synthetic construct
QVQLQESGGGLVQAGGSLRLSCAASGRTSSGYAMGWFRQAPGKEREFVAAIAWSAGSTYYADSVQGRFTISRDKPKIMVYLQMDSLTPEDTAVYYCAAGTRMPSRMTNAYDYWGRGTQVTVSS

SEQ ID NO: 566 - protein, 123 aa, synthetic construct
QVQLQESGGGLVQAGGSLRLSCAASGRTFSSTAMGWFRRPPGKEREFVAAIRWSDYRTDYADSVKDRFTVSRDNAKNTVYLQMNSLKPEDTAVYYCVAGHRLNSRFAEAYDYWGQGTQVTVSS

SEQ ID NO: 567 - protein, 124 aa, synthetic construct
QVQLQESGGGLMQAGGSLRLSCAASGRTVSTYFMSWFRQAPGKEREFVATIGGSGGSRYYADPVQGRFTISRDNAKNTVYLQMTALKPEDTAVYYCATGPAGFGSRKTTSYDYWGQGTQVTVSS

SEQ ID NO: 568 - protein, 123 aa, synthetic construct
QVQLQESGGGLVQAGGSLRLSCAAFGRTFSSTAMGWFRQPPGKEREFVAAIRWSDYRTDYADSVKDRFTVSRDNAKNTVYLQMNSLKPEDTAVYYCVAGHRLNSRFAEAYDYWGQGTQVTVSS

SEQ ID NO: 569 - protein, 123 aa, synthetic construct
QVQLQESGGGLVQAGGSLRLSCAASGRTFSSTAMGWFRQAPGKEREFVAAIRWSDYRTYYADSVKDRFTVSRDNAKNTVYLQMNSLKPEDTAVYYCVAGHRLITRITEAYDYWGQGTQVTVSS

SEQ ID NO: 570 - protein, 113 aa, synthetic construct
QVQLQESGGGLVQSGGSLRLSCVVSGRLISADSMTWYRQAPEKQREFVAHITSGGGTNYADSVQGRFTISRDNAKNTVYLQMNSLKPEDTAVYYCNCPRYGDWGQGTQVTVSS

SEQ ID NO: 571 - protein, 122 aa, synthetic construct
QVQLQESGGGLVQAGDSLRLSCAASGLTFSSYTMAWFRQAPGKEREFVAAASWSGGSTYYADSVEGRFTISRDYAKNTVYLEMNSLKPEDTAVYYCAAEKAPSQTVAAYEYWGQGTQVTVSS

SEQ ID NO: 572 - protein, 113 aa, synthetic construct
QVQLQESGGGLVQSGGSLRLSCVASGRLISANSMGWYRQAPEKQREFVAHITSGGGTNYADSVKGRFTISRDNAKNTVYLQMNSLKPEDTAVYYCNWPRYGDWGQGTQVTVSS

SEQ ID NO: 573 - protein, 126 aa, synthetic construct
QVQLQESGGGLVQAGGSLRLSCAASGRAFRINSIGWFRQAPGKEREFVAAISWSGRDTYYDDSVKGRFTISRDNAKNTVYLEMNSLKPADTAVYSCAARVFFDSGSYAASEYSNWGQGTQVTVFSS

SEQ ID NO: 574 - protein, 124 aa, synthetic construct
QVQLQESGGGLVQAGGSLRLSCAASGRTTSTYFMAWFRQAPGKEREFVATIGGSGGSRHYADPVQGRFTISRDNAKSTMYLQMNALKPEDTAVYYCATGPRGFGSRRSTSYDYWGQGTQVTVSS

SEQ ID NO: 575 - protein, 121 aa, synthetic construct
QVQLQESGGGLVQAGGSLRLSCAASGSSSSINAMAWYRQAPGKQREMVAHISRARGDSTIYADSVKGRFTISRENAKNTVYLQMNNLKPEDTAVYFCYVAGNRDLDFRSYWGQGTQVTVSS

SEQ ID NO: 576 - protein, 124 aa, synthetic construct
QVQLQESGGGLVQAGGSLRLSCSASERTFSTYFEAWFRQTPGKEREFVATIGGNGGSRYYAEPVEGRFFISRDNAKNTVYLEMSALKPEDTAVYYCATGPRGFGSRKSTSYDYWGQGTQVTVSS

SEQ ID NO: 577 - protein, 124 aa, synthetic construct
QVQLQESGGGLVQAGDSLRLSCAASGRTSSTYFMTWFRQAPGKEREFVATIGGSGGSRHYADPVQGRFTISRDNAKSTMYLQMNALKPEDTAVYYCATGPRGFGSRKSTSYDYWGQGTQVTVSS

SEQ ID NO: 578 - protein, 124 aa, synthetic construct
QVQLQESGGGLVQAGGSLRLSCAASGRPFSSYAMGWIRQAPGKEREFVAAIGGDGSTTRYTESAKGRFTISRDNAKNTMYLQMNSLIPEDTAVYYCALDFSLNRIVFGTRADYWGQGTQVTVSS

SEQ ID NO: 579 - protein, 115 aa, synthetic construct
QVQLQESGGGLVQAGGSLRLSCAASATISSTDFMGWYRQAPGKQRELVAGITSSDLTNYADSVKGRFIISRDNAKNTVALQMNSLKPEDTATYYCRVWGLGYYYWGQGTQVTVSS

SEQ ID NO: 580 - protein, 124 aa, synthetic construct
QVQLQESGGGLVQAGGSLRLSCAASGRTFSMYNMGWFRQAPGKEREFVATIGGNGGSRYYADPVEGRFTISRDNAKNTLYLQMNALEPEDTAIYYCATGPRGFGSQKSNSYDYWGQGTQVTVSS

SEQ ID NO: 581 - protein, 124 aa, synthetic construct
QVQLQESGGGLVQAGGSLRLSCAASGRTVSTYFMSWFRQAPGKEREFVATIGGSGGSRHYADPVQGRFTISRDNAKNTVYLQMTALKPEDTAVYYCATGPAGFGSRKTTSYDYWGQGTQVTVSS

SEQ ID NO: 582 - protein, 121 aa, synthetic construct
QVQLQESGGGLVRAGGSLRLSCAASFSSSYMGWFRQAPGKEREFVATIGGNGGSRYYADPVEGRFTISRDNAKNTVYLQMNALRPEDTAVYYCATGPRGFGSRKSTSYDYWGQGTQVTVSS

SEQ ID NO: 583 - protein, 124 aa, synthetic construct
QVQLQESGGGLVQAGGSLRLSCAASGRAFSTYFMAWFRQGPGKEREFVATIGGSGGSRYYADPVEGRFTISRDNAKNTMYLQMNALKPEDTAVYYCATGPRGFGSRKTTSYDYWGQGTQVTVSS

SEQ ID NO: 584 - protein, 123 aa, synthetic construct
QVQLQESGGGLVQAGGSLRLSCGASGRTFSSYAVGWFRQAAGKEREFVGAISWTGGRTTYADSVKGRFTISRDNAKNTLYLQMNSLKPEDTAVYYCTARLLGGGWSGEEYDYWGQGTQVTVSS

SEQ ID NO: 585 - protein, 124 aa, synthetic construct
QVQLQESGGGLVQAGGSLRLSCAASGRSFITYFMAWFRQAPGKEREFVATIGGNGGSRYYADPVEGRFTISRDNAKNTVYLQMNALKPEDTAVYYCATGPRGFGSRKSTSYDYWGQGTQVTVSS

SEQ ID NO: 586 - protein, 123 aa, synthetic construct
QVQLQESGGGLVQAGGSLRLSCGASGRTASSYAMGWFRQAAGKEREFVAAMPWTGGRTTYADSVKGRFTISRDNAKNTVFLQMNSLKPEDTGVYYCAARLLGGGWSGEEYDYWGQGTQVTVSS

SEQ ID NO: 587 - protein, 124 aa, synthetic construct
QVQLQESGGGLVQTGGSLRLSCAASGRSFSTYFMAWFRQFPGKEREFVATIGGNGGSRYYADPVQGRFTISRDNAKNTGYLQMNALQPEDTAVYYCATGPRGFGSRKSTSYDYWGQGTQVTVSS

SEQ ID NO: 588 - protein, 122 aa, synthetic construct
QVQLQESGGGLVRAGGSLRLSCAASGLTFSSYSMGWFRQAPGKEREFVGAISWSGGRTYYADSVTGRFTISRDNAKNTVYLQMNNLKPEDTGVYYCAAERAPSRQVAAYEFWGQGTQVTVSS

SEQ ID NO: 589 - protein, 115 aa, synthetic construct
QVQLQESGGGLVQAGGSLRLSCAASGSISSTHFMGWYRQAPGKQRELVAGITESSLTNYADSVKGRFIISREHAKNTVALQMNSLEPEDTAVYYCGVWGLGYAYWGQGTQVTVSS

SEQ ID NO: 590 - protein, 115 aa, synthetic construct
QVQLQESGGGLVQAGGSLRLSCAASGSIASTHFMGWYRQAPGKQRELVAGLTESSLTNYADSVKGRFIISREHAKNTVALQMNSLKPEDTAVYYCGVWGLGFAYWGQGTQVTVSS

SEQ ID NO: 591 - protein, 115 aa, synthetic construct
QVQLQESGGGLVQAGGSLRLSCAASGSIFSTNIMGWYRQAPGKQRELVAGITGSDLTNYADSVKGRFIISRDNAPNTVALRMNSLKPEDTATYYCRVWGLGYYYWGRGTQVTVSS

SEQ ID NO: 592 - protein, 124 aa, synthetic construct
QVQLQESGGGLVQAGGSLRLSCAASGGTFSTYFMTWFRQAPGKEREFVATIGGSGGSRHYADPVQGRFTISRDNAKSTMYLQMNALEPEDTAVYYCATGPRGFGSRKSTSYDYWGQGTQVTVSS

SEQ ID NO: 593 - protein, 115 aa, synthetic construct
QVQLQESGGGLVQAGGSLRLSCTASGSISSTHFMGWYRQAPGNQRELVAGITTSSATSYADSVKGRFIISREHAKNTVALQMNSLKPEDTAVYYCGVWGVGYAYWGQGTQVTVSS

SEQ ID NO: 594 - protein, 117 aa, synthetic construct
QVQLQESGGGLVQAGGSLRLSCAASGYISSTHFMGWYRQAPGKQRELVAGITGSDLTNYADSVKGRFIISRDNAKNTVALEMNSLKPEDTAVYYCRLWGLGLGYGYWGQGTQVTVSS

SEQ ID NO: 595 - protein, 124 aa, synthetic construct
QVQLQESGGGLVQAGGSLRLSCAASGRPFSSYAMGWFRQAPGKEREFVAAIGGDGSITQYTESAKGRFTISRDNAKNTIYLQMNSLKPEDTAVYVCALDFSFNRIVLGTRADYWGQGTQVTVSS

SEQ ID NO: 596 - protein, 124 aa, synthetic construct
QVQLQESGGGLVQAGGSLRLSCSASERTFSTYFKAWFRQAPGKGREFVATIGGNGGSRYYADPVKGRFFISRDNAKNTVYLEMNALKPEDTAVYYCATGPRGFGSRKSTSYDYWGQGTQVTVSS

SEQ ID NO: 597 - protein, 126 aa, synthetic construct
QVQLQESGGGLVQAGGSLRLSCAASGRTFSNYDLGWFRQAPGKEREFIAACGWKAEDTYYLNSVKGRFTISRDNAKNTVTLQMNSLNPEDTAIYYCAAGLTVNGRLLTRTYEYDIWGQGTQVTVSS

SEQ ID NO: 598 - protein, 124 aa, synthetic construct
QVQLQESGGGLVQAGGSLRLSCAASGRTVSTYFMSWFRQAPGKEREFVATIGGSGGSRYYADPVKGRFTISRDNAKNTVYLQMTALEPEDTAVYYCATGPAGFGSRKSTSYDYWGQGTQVTVSS

SEQ ID NO: 599 - protein, 124 aa, synthetic construct
QVQLQESGGGLVQAGGSLRLSCAASGRSFSTYFMAWFRQAPGKEREFVATIGGNGGSRYYADPVEGRFTISRDNAKNTLYLQMNALEPEDTAIYYCATGPRGFGSRKSNSYDYWGQGTQVTVSS

SEQ ID NO: 600 - protein, 124 aa, synthetic construct
QVQLQESGGGLVQAGGSLRLSCAASGRSFSTYFMAWFRQAPGKEREFVATIGGNGGSRYYADPVEGRFTISRDNAKNTVYLQMNALKPEDTAVYYCATGPRGFGSRKSTSYDYWGQGTQVTVSS

SEQ ID NO: 601 - protein, 122 aa, synthetic construct
QVQLQESGGGLVQAGGSLRLSCAASGGTYTTYTMGWFRQAPGKDRTIVAAIRRTGGDPYYSTSVKGRFTISRDNAKNTVYLQMNSLKPEDTAVYYCAGSPLWTSSQDDYRHWGQGTQVTVSS

SEQ ID NO: 602 - protein, 124 aa, synthetic construct
QVQLQESGGGLVQAGGSLRLSCSASERTFSTYFKVWFRQAPGKEREFVATIGGNGGSRYYADSVEGRFFISRDNAKNTVYLEMNALKPEDTAVYYCATGPRGFGSRKSTSYDYWGQGTQVTVSS

SEQ ID NO: 603 - protein, 115 aa, synthetic construct
QVQLQESGGGLVQAGGSLGLSCAAYGSIPSTHFMGWYRQPPGKQRELVAGITGSDVTNYADSVKGRFIISREHAQNTVALQMNSLKPEDTAVYYCRVWGVGYDYWGQGTQVTVSS

SEQ ID NO: 604 - protein, 115 aa, synthetic construct
QVQPQESGGGLVQAGGSLGLSCAASGSIPSTHFMGWYRQPPGKQRELVAGITGSDLTNYADSVKGRFIISREHAQNTVALQMNSLKPEDTAVYYCRVWGVGYDYWGQGTQVTVSS

(19) United States
(12) Patent Application Publication
(10) Pub. No.: US 2025/0340642 A1
(43) Pub. Date: Nov. 6, 2025

(54) ANTI-CD28 ANTIBODIES

(71) Applicant: SANOFI, Paris (FR)

(72) Inventors: Klervi DESRUMEAUX, Paris (FR); Charlotte LAHOUTE, Paris (FR); Emmanuelle VIGNE, Paris (FR)

(21) Appl. No.: 19/079,824

(22) Filed: Mar. 14, 2025

Related U.S. Application Data
(63) Continuation of application No. PCT/EP2023/077092, filed on Sep. 29, 2023.

(30) Foreign Application Priority Data
Sep. 30, 2022 (EP) 22315222.4

(51) Int. Cl. C07K 16/28; A61P 37/04

(52) U.S. Cl. CPC C07K 16/2818; A61P 37/04; C07K 2317/24; C07K 2317/31; C07K 2317/522; C07K 2317/565; C07K 2317/569; C07K 2317/76

(57) ABSTRACT

Antibodies and antigen-binding fragments thereof that bind to CD28, and methods of using the same, are provided.

### RELATED APPLICATIONS

This application is a continuation of International Patent Application No. PCT/EP2023/077092, filed Sep. 29, 2023, which claims priority to European Patent Application No. 22315222.4, filed Sep. 30, 2022, the disclosures of which are hereby incorporated by reference in their entirety.

### SEQUENCE LISTING

The content of the electronically submitted sequence listing in XML format (Name: 762927_SA9-345PCCON_ST26.xml; Size: 541,756 bytes; and Date of Creation: Mar. 13, 2025) is incorporated herein by reference in its entirety.

### FIELD OF THE INVENTION

This disclosure relates to novel antibodies and antigen-binding fragments thereof that specifically bind to CD28, and methods of using the same.

### BACKGROUND

The cluster of differentiation 28 (CD28) is a member of costimulatory proteins expressed on the surface of T-receptor cells. Since costimulatory receptors can regulate T-cell activation and thus the course of a specific immune response, they have been tested to control T-cell responses in both oncology and inflammation settings. As such, CD28 antagonists have been investigated for treating autoimmune and inflammatory diseases and CD28 agonists have been studied for oncology diseases. However, the administration of agonistic anti-CD28 antibodies has been associated with severe systemic inflammatory responses, including cytokine storm. These dangerous inflammatory side effects have severely limited the therapeutic window of and the therapeutic use of anti-CD28 antibodies. Therefore, there is a need for novel anti-CD28 antibodies that are not associated with severe inflammatory responses.

### SUMMARY

The subject specification provides anti-CD28 antibodies (e.g., anti-CD28 VHH antibodies) and antigen-binding fragments thereof. Methods of inhibiting the activity of CD28 or treating a CD28-associated disease are also provided.

In one aspect, the disclosure provides an antibody or antigen-binding fragment thereof that specifically binds to CD28, comprising an immunoglobulin single variable domain (ISVD), wherein the ISVD domain comprises a CDR-H1 amino acid sequence, a CDR-H2 amino acid sequence, and a CDR-H3 amino acid sequence selected from any one of the CDR-H3, CDR-H2, and CDR-H3 amino acid sequences of Table 1.

In certain embodiments, the ISVD domain comprises any one of the ISVD amino acid sequences of Table 2.

In another aspect, the disclosure provides an antibody or antigen-binding fragment thereof that specifically binds to CD28, comprising an immunoglobulin single variable domain (ISVD) comprising a CDR-H1 sequence, a CDR-H2 sequence, and a CDR-H3 sequence, wherein:
a) the CDR-H1 sequence comprises an amino acid sequence set forth in SEQ ID NO: 1, the CDR-H2 sequence comprises an amino acid sequence set forth in SEQ ID NO: 2, and the CDR-H3 sequence comprises an amino acid sequence set forth in SEQ ID NO: 3;
b) the CDR-H1 sequence comprises an amino acid sequence set forth in SEQ ID NO: 4, the CDR-H2 sequence comprises an amino acid sequence set forth in SEQ ID NO: 5, and the CDR-H3 sequence comprises an amino acid sequence set forth in SEQ ID NO: 6;
c) the CDR-H1 sequence comprises an amino acid sequence set forth in SEQ ID NO: 7, the CDR-H2 sequence comprises an amino acid sequence set forth in SEQ ID NO: 8, and the CDR-H3 sequence comprises an amino acid sequence set forth in SEQ ID NO: 9;
d) the CDR-H1 sequence comprises an amino acid sequence set forth in SEQ ID NO: 10, the CDR-H2 sequence comprises an amino acid sequence set forth in SEQ ID NO: 11, and the CDR-H3 sequence comprises an amino acid sequence set forth in SEQ ID NO: 12;
e) the CDR-H1 sequence comprises an amino acid sequence set forth in SEQ ID NO: 13, the CDR-H2 sequence comprises an amino acid sequence set forth in SEQ ID NO: 14, and the CDR-H3 sequence comprises an amino acid sequence set forth in SEQ ID NO: 15;
f) the CDR-H1 sequence comprises an amino acid sequence set forth in SEQ ID NO: 16, the CDR-H2 sequence comprises an amino acid sequence set forth in SEQ ID NO: 17, and the CDR-H3 sequence comprises an amino acid sequence set forth in SEQ ID NO: 18;
g) the CDR-H1 sequence comprises an amino acid sequence set forth in SEQ ID NO: 19, the CDR-H2 sequence comprises an amino acid sequence set forth in SEQ ID NO: 20, and the CDR-H3 sequence comprises an amino acid sequence set forth in SEQ ID NO: 21;
h) the CDR-H1 sequence comprises an amino acid sequence set forth in SEQ ID NO: 22, the CDR-H2 sequence comprises an amino acid sequence set forth in SEQ ID NO: 23, and the CDR-H3 sequence comprises an amino acid sequence set forth in SEQ ID NO: 24;
i) the CDR-H1 sequence comprises an amino acid sequence set forth in SEQ ID NO: 25, the CDR-H2 sequence comprises an amino acid sequence set forth in SEQ ID NO: 26, and the CDR-H3 sequence comprises an amino acid sequence set forth in SEQ ID NO: 27;
j) the CDR-H1 sequence comprises an amino acid sequence set forth in SEQ ID NO: 28, the CDR-H2 sequence comprises an amino acid sequence set forth in SEQ ID NO: 29, and the CDR-H3 sequence comprises an amino acid sequence set forth in SEQ ID NO: 30;
k) the CDR-H1 sequence comprises an amino acid sequence set forth in SEQ ID NO: 31, the CDR-H2 sequence comprises an amino acid sequence set forth in SEQ ID NO: 32, and the CDR-H3 sequence comprises an amino acid sequence set forth in SEQ ID NO: 33;
l) the CDR-H1 sequence comprises an amino acid sequence set forth in SEQ ID NO: 34, the CDR-H2 sequence comprises an amino acid sequence set forth in SEQ ID NO: 35, and the CDR-H3 sequence comprises an amino acid sequence set forth in SEQ ID NO: 36;
m) the CDR-H1 sequence comprises an amino acid sequence set forth in SEQ ID NO: 37, the CDR-H2 sequence comprises an amino acid sequence set forth in SEQ ID NO: 38, and the CDR-H3 sequence comprises an amino acid sequence set forth in SEQ ID NO: 39;
n) the CDR-H1 sequence comprises an amino acid sequence set forth in SEQ ID NO: 40, the CDR-H2 sequence comprises an amino acid sequence set forth in SEQ ID NO: 41, and the CDR-H3 sequence comprises an amino acid sequence set forth in SEQ ID NO: 42;
o) the CDR-H1 sequence comprises an amino acid sequence set forth in SEQ ID NO: 43, the CDR-H2 sequence comprises an amino acid sequence set forth in SEQ ID NO: 44, and the CDR-H3 sequence comprises an amino acid sequence set forth in SEQ ID NO: 45;
p) the CDR-H1 sequence comprises an amino acid sequence set forth in SEQ ID NO: 46, the CDR-H2 sequence comprises an amino acid sequence set forth in SEQ ID NO: 47, and the CDR-H3 sequence comprises an amino acid sequence set forth in SEQ ID NO: 48;
q) the CDR-H1 sequence comprises an amino acid sequence set forth in SEQ ID NO: 49, the CDR-H2 sequence comprises an amino acid sequence set forth in SEQ ID NO: 50, and the CDR-H3 sequence comprises an amino acid sequence set forth in SEQ ID NO: 51;
r) the CDR-H1 sequence comprises an amino acid sequence set forth in SEQ ID NO: 52, the CDR-H2 sequence comprises an amino acid sequence set forth in SEQ ID NO: 53, and the CDR-H3 sequence comprises an amino acid sequence set forth in SEQ ID NO: 54;
s) the CDR-H1 sequence comprises an amino acid sequence set forth in SEQ ID NO: 55, the CDR-H2 sequence comprises an amino acid sequence set forth in SEQ ID NO: 56, and the CDR-H3 sequence comprises an amino acid sequence set forth in SEQ ID NO: 57;
t) the CDR-H1 sequence comprises an amino acid sequence set forth in SEQ ID NO: 58, the CDR-H2 sequence comprises an amino acid sequence set forth in SEQ ID NO: 59, and the CDR-H3 sequence comprises an amino acid sequence set forth in SEQ ID NO: 60;
u) the CDR-H1 sequence comprises an amino acid sequence set forth in SEQ ID NO: 61, the CDR-H2 sequence comprises an amino acid sequence set forth in SEQ ID NO: 62, and the CDR-H3 sequence comprises an amino acid sequence set forth in SEQ ID NO: 63;
v) the CDR-H1 sequence comprises an amino acid sequence set forth in SEQ ID NO: 64, the CDR-H2 sequence comprises an amino acid sequence set forth in SEQ ID NO: 65, and the CDR-H3 sequence comprises an amino acid sequence set forth in SEQ ID NO: 66;
w) the CDR-H1 sequence comprises an amino acid sequence set forth in SEQ ID NO: 67, the CDR-H2 sequence comprises an amino acid sequence set forth in SEQ ID NO: 68, and the CDR-H3 sequence comprises an amino acid sequence set forth in SEQ ID NO: 69;
x) the CDR-H1 sequence comprises an amino acid sequence set forth in SEQ ID NO: 70, the CDR-H2 sequence comprises an amino acid sequence set forth in SEQ ID NO: 71, and the CDR-H3 sequence comprises an amino acid sequence set forth in SEQ ID NO: 72;
y) the CDR-H1 sequence comprises an amino acid sequence set forth in SEQ ID NO: 73, the CDR-H2 sequence comprises an amino acid sequence set forth in SEQ ID NO: 74, and the CDR-H3 sequence comprises an amino acid sequence set forth in SEQ ID NO: 75;
z) the CDR-H1 sequence comprises an amino acid sequence set forth in SEQ ID NO: 76, the CDR-H2 sequence comprises an amino acid sequence set forth in SEQ ID NO: 77, and the CDR-H3 sequence comprises an amino acid sequence set forth in SEQ ID NO: 78;
aa) the CDR-H1 sequence comprises an amino acid sequence set forth in SEQ ID NO: 79, the CDR-H2 sequence comprises an amino acid sequence set forth in SEQ ID NO: 80, and the CDR-H3 sequence comprises an amino acid sequence set forth in SEQ ID NO: 81;
ab) the CDR-H1 sequence comprises an amino acid sequence set forth in SEQ ID NO: 82, the CDR-H2 sequence comprises an amino acid sequence set forth in SEQ ID NO: 83, and the CDR-H3 sequence comprises an amino acid sequence set forth in SEQ ID NO: 84;
ac) the CDR-H1 sequence comprises an amino acid sequence set forth in SEQ ID NO: 85, the CDR-H2 sequence comprises an amino acid sequence set forth in SEQ ID NO: 86, and the CDR-H3 sequence comprises an amino acid sequence set forth in SEQ ID NO: 87;
ad) the CDR-H1 sequence comprises an amino acid sequence set forth in SEQ ID NO: 88, the CDR-H2 sequence comprises an amino acid sequence set forth in SEQ ID NO: 89, and the CDR-H3 sequence comprises an amino acid sequence set forth in SEQ ID NO: 90;
ae) the CDR-H1 sequence comprises an amino acid sequence set forth in SEQ ID NO: 91, the CDR-H2 sequence comprises an amino acid sequence set forth in SEQ ID NO: 92, and the CDR-H3 sequence comprises an amino acid sequence set forth in SEQ ID NO: 93;
af) the CDR-H1 sequence comprises an amino acid sequence set forth in SEQ ID NO: 94, the CDR-H2 sequence comprises an amino acid sequence set forth in SEQ ID NO: 95, and the CDR-H3 sequence comprises an amino acid sequence set forth in SEQ ID NO: 96;
ag) the CDR-H1 sequence comprises an amino acid sequence set forth in SEQ ID NO: 97, the CDR-H2 sequence comprises an amino acid sequence set forth in SEQ ID NO: 98, and the CDR-H3 sequence comprises an amino acid sequence set forth in SEQ ID NO: 99;
ah) the CDR-H1 sequence comprises an amino acid sequence set forth in SEQ ID NO: 100, the CDR-H2 sequence comprises an amino acid sequence set forth in SEQ ID NO: 101, and the CDR-H3 sequence comprises an amino acid sequence set forth in SEQ ID NO: 102;
ai) the CDR-H1 sequence comprises an amino acid sequence set forth in SEQ ID NO: 103, the CDR-H2 sequence comprises an amino acid sequence set forth in SEQ ID NO: 104, and the CDR-H3 sequence comprises an amino acid sequence set forth in SEQ ID NO: 105;
aj) the CDR-H1 sequence comprises an amino acid sequence set forth in SEQ ID NO: 106, the CDR-H2 sequence comprises an amino acid sequence set forth in SEQ ID NO: 107, and the CDR-H3 sequence comprises an amino acid sequence set forth in SEQ ID NO: 108;
ak) the CDR-H1 sequence comprises an amino acid sequence set forth in SEQ ID NO: 109, the CDR-H2 sequence comprises an amino acid sequence set forth in SEQ ID NO: 110, and the CDR-H3 sequence comprises an amino acid sequence set forth in SEQ ID NO: 111;
al) the CDR-H1 sequence comprises an amino acid sequence set forth in SEQ ID NO: 112, the CDR-H2 sequence comprises an amino acid sequence set forth in SEQ ID NO: 113, and the CDR-H3 sequence comprises an amino acid sequence set forth in SEQ ID NO: 114;
am) the CDR-H1 sequence comprises an amino acid sequence set forth in SEQ ID NO: 115, the CDR-H2 sequence comprises an amino acid sequence set forth in SEQ ID NO: 116, and the CDR-H3 sequence comprises an amino acid sequence set forth in SEQ ID NO: 117;
an) the CDR-H1 sequence comprises an amino acid sequence set forth in SEQ ID NO: 118, the CDR-H2 sequence comprises an amino acid sequence set forth in SEQ ID NO: 119, and the CDR-H3 sequence comprises an amino acid sequence set forth in SEQ ID NO: 120;
ao) the CDR-H1 sequence comprises an amino acid sequence set forth in SEQ ID NO: 121, the CDR-H2 sequence comprises an amino acid sequence set forth in SEQ ID NO: 122, and the CDR-H3 sequence comprises an amino acid sequence set forth in SEQ ID NO: 123;
ap) the CDR-H1 sequence comprises an amino acid sequence set forth in SEQ ID NO: 124, the CDR-H2 sequence comprises an amino acid sequence set forth in SEQ ID NO: 125, and the CDR-H3 sequence comprises an amino acid sequence set forth in SEQ ID NO: 126;
aq) the CDR-H1 sequence comprises an amino acid sequence set forth in SEQ ID NO: 127, the CDR-H2 sequence comprises an amino acid sequence set forth in SEQ ID NO: 128, and the CDR-H3 sequence comprises an amino acid sequence set forth in SEQ ID NO: 129;
ar) the CDR-H1 sequence comprises an amino acid sequence set forth in SEQ ID NO: 130, the CDR-H2 sequence comprises an amino acid sequence set forth in SEQ ID NO: 131, and the CDR-H3 sequence comprises an amino acid sequence set forth in SEQ ID NO: 132;
as) the CDR-H1 sequence comprises an amino acid sequence set forth in SEQ ID NO: 133, the CDR-H2 sequence comprises an amino acid sequence set forth in SEQ ID NO: 134, and the CDR-H3 sequence comprises an amino acid sequence set forth in SEQ ID NO: 135;
at) the CDR-H1 sequence comprises an amino acid sequence set forth in SEQ ID NO: 136, the CDR-H2 sequence comprises an amino acid sequence set forth in SEQ ID NO: 137, and the CDR-H3 sequence comprises an amino acid sequence set forth in SEQ ID NO: 138;
au) the CDR-H1 sequence comprises an amino acid sequence set forth in SEQ ID NO: 139, the CDR-H2 sequence comprises an amino acid sequence set forth in SEQ ID NO: 140, and the CDR-H3 sequence comprises an amino acid sequence set forth in SEQ ID NO: 141;
av) the CDR-H1 sequence comprises an amino acid sequence set forth in SEQ ID NO: 142, the CDR-H2 sequence comprises an amino acid sequence set forth in SEQ ID NO: 143, and the CDR-H3 sequence comprises an amino acid sequence set forth in SEQ ID NO: 144;
aw) the CDR-H1 sequence comprises an amino acid sequence set forth in SEQ ID NO: 145, the CDR-H2 sequence comprises an amino acid sequence set forth in SEQ ID NO: 146, and the CDR-H3 sequence comprises an amino acid sequence set forth in SEQ ID NO: 147;
ax) the CDR-H1 sequence comprises an amino acid sequence set forth in SEQ ID NO: 148, the CDR-H2 sequence comprises an amino acid sequence set forth in SEQ ID NO: 149, and the CDR-H3 sequence comprises an amino acid sequence set forth in SEQ ID NO: 150;
ay) the CDR-H1 sequence comprises an amino acid sequence set forth in SEQ ID NO: 151, the CDR-H2 sequence comprises an amino acid sequence set forth in SEQ ID NO: 152, and the CDR-H3 sequence comprises an amino acid sequence set forth in SEQ ID NO: 153;
az) the CDR-H1 sequence comprises an amino acid sequence set forth in SEQ ID NO: 154, the CDR-H2 sequence comprises an amino acid sequence set forth in SEQ ID NO: 155, and the CDR-H3 sequence comprises an amino acid sequence set forth in SEQ ID NO: 156;
ba) the CDR-H1 sequence comprises an amino acid sequence set forth in SEQ ID NO: 157, the CDR-H2 sequence comprises an amino acid sequence set forth in SEQ ID NO: 158, and the CDR-H3 sequence comprises an amino acid sequence set forth in SEQ ID NO: 159;
bb) the CDR-H1 sequence comprises an amino acid sequence set forth in SEQ ID NO: 160, the CDR-H2 sequence comprises an amino acid sequence set forth in SEQ ID NO: 161, and the CDR-H3 sequence comprises an amino acid sequence set forth in SEQ ID NO: 162;
bc) the CDR-H1 sequence comprises an amino acid sequence set forth in SEQ ID NO: 163, the CDR-H2 sequence comprises an amino acid sequence set forth in SEQ ID NO: 164, and the CDR-H3 sequence comprises an amino acid sequence set forth in SEQ ID NO: 165;
bd) the CDR-H1 sequence comprises an amino acid sequence set forth in SEQ ID NO: 166, the CDR-H2 sequence comprises an amino acid sequence set forth in SEQ ID NO: 167, and the CDR-H3 sequence comprises an amino acid sequence set forth in SEQ ID NO: 168;
be) the CDR-H1 sequence comprises an amino acid sequence set forth in SEQ ID NO: 169, the CDR-H2 sequence comprises an amino acid sequence set forth in SEQ ID NO: 170, and the CDR-H3 sequence comprises an amino acid sequence set forth in SEQ ID NO: 171;
bf) the CDR-H1 sequence comprises an amino acid sequence set forth in SEQ ID NO: 172, the CDR-H2 sequence comprises an amino acid sequence set forth in SEQ ID NO: 173, and the CDR-H3 sequence comprises an amino acid sequence set forth in SEQ ID NO: 174;
bg) the CDR-H1 sequence comprises an amino acid sequence set forth in SEQ ID NO: 175, the CDR-H2 sequence comprises an amino acid sequence set forth in SEQ ID NO: 176, and the CDR-H3 sequence comprises an amino acid sequence set forth in SEQ ID NO: 177;
bh) the CDR-H1 sequence comprises an amino acid sequence set forth in SEQ ID NO: 178, the CDR-H2 sequence comprises an amino acid sequence set forth in SEQ ID NO: 179, and the CDR-H3 sequence comprises an amino acid sequence set forth in SEQ ID NO: 180;
bi) the CDR-H1 sequence comprises an amino acid sequence set forth in SEQ ID NO: 181, the CDR-H2 sequence comprises an amino acid sequence set forth in SEQ ID NO: 182, and the CDR-H3 sequence comprises an amino acid sequence set forth in SEQ ID NO: 183;
bj) the CDR-H1 sequence comprises an amino acid sequence set forth in SEQ ID NO: 184, the CDR-H2 sequence comprises an amino acid sequence set forth in SEQ ID NO: 185, and the CDR-H3 sequence comprises an amino acid sequence set forth in SEQ ID NO: 186;
bk) the CDR-H1 sequence comprises an amino acid sequence set forth in SEQ ID NO: 187, the CDR-H2 sequence comprises an amino acid sequence set forth in SEQ ID NO: 188, and the CDR-H3 sequence comprises an amino acid sequence set forth in SEQ ID NO: 189;
bl) the CDR-H1 sequence comprises an amino acid sequence set forth in SEQ ID NO: 190, the CDR-H2 sequence comprises an amino acid sequence set forth in SEQ ID NO: 191, and the CDR-H3 sequence comprises an amino acid sequence set forth in SEQ ID NO: 192;
bm) the CDR-H1 sequence comprises an amino acid sequence set forth in SEQ ID NO: 193, the CDR-H2 sequence comprises an amino acid sequence set forth in SEQ ID NO: 194, and the CDR-H3 sequence comprises an amino acid sequence set forth in SEQ ID NO: 195;
bn) the CDR-H1 sequence comprises an amino acid sequence set forth in SEQ ID NO: 196, the CDR-H2 sequence comprises an amino acid sequence set forth in SEQ ID NO: 197, and the CDR-H3 sequence comprises an amino acid sequence set forth in SEQ ID NO: 198;
bo) the CDR-H1 sequence comprises an amino acid sequence set forth in SEQ ID NO: 199, the CDR-H2 sequence comprises an amino acid sequence set forth in SEQ ID NO: 200, and the CDR-H3 sequence comprises an amino acid sequence set forth in SEQ ID NO: 201;
bp) the CDR-H1 sequence comprises an amino acid sequence set forth in SEQ ID NO: 202, the CDR-H2 sequence comprises an amino acid sequence set forth in SEQ ID NO: 203, and the CDR-H3 sequence comprises an amino acid sequence set forth in SEQ ID NO: 204;
bq) the CDR-H1 sequence comprises an amino acid sequence set forth in SEQ ID NO: 205, the CDR-H2 sequence comprises an amino acid sequence set forth in SEQ ID NO: 206, and the CDR-H3 sequence comprises an amino acid sequence set forth in SEQ ID NO: 207;
br) the CDR-H1 sequence comprises an amino acid sequence set forth in SEQ ID NO: 208, the CDR-H2 sequence comprises an amino acid sequence set forth in SEQ ID NO: 209, and the CDR-H3 sequence comprises an amino acid sequence set forth in SEQ ID NO: 210;
bs) the CDR-H1 sequence comprises an amino acid sequence set forth in SEQ ID NO: 211, the CDR-H2 sequence comprises an amino acid sequence set forth in SEQ ID NO: 212, and the CDR-H3 sequence comprises an amino acid sequence set forth in SEQ ID NO: 213;
bt) the CDR-H1 sequence comprises an amino acid sequence set forth in SEQ ID NO: 214, the CDR-H2 sequence comprises an amino acid sequence set forth in SEQ ID NO: 215, and the CDR-H3 sequence comprises an amino acid sequence set forth in SEQ ID NO: 216;
bu) the CDR-H1 sequence comprises an amino acid sequence set forth in SEQ ID NO: 217, the CDR-H2 sequence comprises an amino acid sequence set forth in SEQ ID NO: 218, and the CDR-H3 sequence comprises an amino acid sequence set forth in SEQ ID NO: 219;
by) the CDR-H1 sequence comprises an amino acid sequence set forth in SEQ ID NO: 220, the CDR-H2 sequence comprises an amino acid sequence set forth in SEQ ID NO: 221, and the CDR-H3 sequence comprises an amino acid sequence set forth in SEQ ID NO: 222;
bw) the CDR-H1 sequence comprises an amino acid sequence set forth in SEQ ID NO: 223, the CDR-H2 sequence comprises an amino acid sequence set forth in SEQ ID NO: 224, and the CDR-H3 sequence comprises an amino acid sequence set forth in SEQ ID NO: 225;
bx) the CDR-H1 sequence comprises an amino acid sequence set forth in SEQ ID NO: 226, the CDR-H2 sequence comprises an amino acid sequence set forth in SEQ ID NO: 227, and the CDR-H3 sequence comprises an amino acid sequence set forth in SEQ ID NO: 228;
by) the CDR-H1 sequence comprises an amino acid sequence set forth in SEQ ID NO: 229, the CDR-H2 sequence comprises an amino acid sequence set forth in SEQ ID NO: 230, and the CDR-H3 sequence comprises an amino acid sequence set forth in SEQ ID NO: 231;
bz) the CDR-H1 sequence comprises an amino acid sequence set forth in SEQ ID NO: 232, the CDR-H2 sequence comprises an amino acid sequence set forth in SEQ ID NO: 233, and the CDR-H3 sequence comprises an amino acid sequence set forth in SEQ ID NO: 234;
ca) the CDR-H1 sequence comprises an amino acid sequence set forth in SEQ ID NO: 235, the CDR-H2 sequence comprises an amino acid sequence set forth in SEQ ID NO: 236, and the CDR-H3 sequence comprises an amino acid sequence set forth in SEQ ID NO: 237;
cb) the CDR-H1 sequence comprises an amino acid sequence set forth in SEQ ID NO: 238, the CDR-H2 sequence comprises an amino acid sequence set forth in SEQ ID NO: 239, and the CDR-H3 sequence comprises an amino acid sequence set forth in SEQ ID NO: 240;
cc) the CDR-H1 sequence comprises an amino acid sequence set forth in SEQ ID NO: 241, the CDR-H2 sequence comprises an amino acid sequence set forth in SEQ ID NO: 242, and the CDR-H3 sequence comprises an amino acid sequence set forth in SEQ ID NO: 243;
cd) the CDR-H1 sequence comprises an amino acid sequence set forth in SEQ ID NO: 244, the CDR-H2 sequence comprises an amino acid sequence set forth in SEQ ID NO: 245, and the CDR-H3 sequence comprises an amino acid sequence set forth in SEQ ID NO: 246;
ce) the CDR-H1 sequence comprises an amino acid sequence set forth in SEQ ID NO: 247, the CDR-H2 sequence comprises an amino acid sequence set forth in SEQ ID NO: 248, and the CDR-H3 sequence comprises an amino acid sequence set forth in SEQ ID NO: 249;
cf) the CDR-H1 sequence comprises an amino acid sequence set forth in SEQ ID NO: 250, the CDR-H2 sequence comprises an amino acid sequence set forth in SEQ ID NO: 251, and the CDR-H3 sequence comprises an amino acid sequence set forth in SEQ ID NO: 252;
cg) the CDR-H1 sequence comprises an amino acid sequence set forth in SEQ ID NO: 253, the CDR-H2 sequence comprises an amino acid sequence set forth in SEQ ID NO: 254, and the CDR-H3 sequence comprises an amino acid sequence set forth in SEQ ID NO: 255;
ch) the CDR-H1 sequence comprises an amino acid sequence set forth in SEQ ID NO: 256, the CDR-H2 sequence comprises an amino acid sequence set forth in SEQ ID NO: 257, and the CDR-H3 sequence comprises an amino acid sequence set forth in SEQ ID NO: 258;
ci) the CDR-H1 sequence comprises an amino acid sequence set forth in SEQ ID NO: 259, the CDR-H2 sequence comprises an amino acid sequence set forth in SEQ ID NO: 260, and the CDR-H3 sequence comprises an amino acid sequence set forth in SEQ ID NO: 261;
cj) the CDR-H1 sequence comprises an amino acid sequence set forth in SEQ ID NO: 262, the CDR-H2 sequence comprises an amino acid sequence set forth in SEQ ID NO: 263, and the CDR-H3 sequence comprises an amino acid sequence set forth in SEQ ID NO: 264;
ck) the CDR-H1 sequence comprises an amino acid sequence set forth in SEQ ID NO: 265, the CDR-H2 sequence comprises an amino acid sequence set forth in SEQ ID NO: 266, and the CDR-H3 sequence comprises an amino acid sequence set forth in SEQ ID NO: 267;
cl) the CDR-H1 sequence comprises an amino acid sequence set forth in SEQ ID NO: 268, the CDR-H2 sequence comprises an amino acid sequence set forth in SEQ ID NO: 269, and the CDR-H3 sequence comprises an amino acid sequence set forth in SEQ ID NO: 270;
cm) the CDR-H1 sequence comprises an amino acid sequence set forth in SEQ ID NO: 271, the CDR-H2 sequence comprises an amino acid sequence set forth in SEQ ID NO: 272, and the CDR-H3 sequence comprises an amino acid sequence set forth in SEQ ID NO: 273;
cn) the CDR-H1 sequence comprises an amino acid sequence set forth in SEQ ID NO: 274, the CDR-H2 sequence comprises an amino acid sequence set forth in SEQ ID NO: 275, and the CDR-H3 sequence comprises an amino acid sequence set forth in SEQ ID NO: 276;
co) the CDR-H1 sequence comprises an amino acid sequence set forth in SEQ ID NO: 277, the CDR-H2 sequence comprises an amino acid sequence set forth in SEQ ID NO: 278, and the CDR-H3 sequence comprises an amino acid sequence set forth in SEQ ID NO: 279;
cp) the CDR-H1 sequence comprises an amino acid sequence set forth in SEQ ID NO: 280, the CDR-H2 sequence comprises an amino acid sequence set forth in SEQ ID NO: 281, and the CDR-H3 sequence comprises an amino acid sequence set forth in SEQ ID NO: 282;
cq) the CDR-H1 sequence comprises an amino acid sequence set forth in SEQ ID NO: 283, the CDR-H2 sequence comprises an amino acid sequence set forth in SEQ ID NO: 284, and the CDR-H3 sequence comprises an amino acid sequence set forth in SEQ ID NO: 285;
cr) the CDR-H1 sequence comprises an amino acid sequence set forth in SEQ ID NO: 286, the CDR-H2 sequence comprises an amino acid sequence set forth in SEQ ID NO: 287, and the CDR-H3 sequence comprises an amino acid sequence set forth in SEQ ID NO: 288;
cs) the CDR-H1 sequence comprises an amino acid sequence set forth in SEQ ID NO: 289, the CDR-H2 sequence comprises an amino acid sequence set forth in SEQ ID NO: 290, and the CDR-H3 sequence comprises an amino acid sequence set forth in SEQ ID NO: 291;
ct) the CDR-H1 sequence comprises an amino acid sequence set forth in SEQ ID NO: 292, the CDR-H2 sequence comprises an amino acid sequence set forth in SEQ ID NO: 293, and the CDR-H3 sequence comprises an amino acid sequence set forth in SEQ ID NO: 294;
cu) the CDR-H1 sequence comprises an amino acid sequence set forth in SEQ ID NO: 295, the CDR-H2 sequence comprises an amino acid sequence set forth in SEQ ID NO: 296, and the CDR-H3 sequence comprises an amino acid sequence set forth in SEQ ID NO: 297;
cv) the CDR-H1 sequence comprises an amino acid sequence set forth in SEQ ID NO: 298, the CDR-H2 sequence comprises an amino acid sequence set forth in SEQ ID NO: 299, and the CDR-H3 sequence comprises an amino acid sequence set forth in SEQ ID NO: 300;
cw) the CDR-H1 sequence comprises an amino acid sequence set forth in SEQ ID NO: 301, the CDR-H2 sequence comprises an amino acid sequence set forth in SEQ ID NO: 302, and the CDR-H3 sequence comprises an amino acid sequence set forth in SEQ ID NO: 303;
cx) the CDR-H1 sequence comprises an amino acid sequence set forth in SEQ ID NO: 304, the CDR-H2 sequence comprises an amino acid sequence set forth in SEQ ID NO: 305, and the CDR-H3 sequence comprises an amino acid sequence set forth in SEQ ID NO: 306;
cy) the CDR-H1 sequence comprises an amino acid sequence set forth in SEQ ID NO: 307, the CDR-H2 sequence comprises an amino acid sequence set forth in SEQ ID NO: 308, and the CDR-H3 sequence comprises an amino acid sequence set forth in SEQ ID NO: 309;
cz) the CDR-H1 sequence comprises an amino acid sequence set forth in SEQ ID NO: 310, the CDR-H2 sequence comprises an amino acid sequence set forth in SEQ ID NO: 311, and the CDR-H3 sequence comprises an amino acid sequence set forth in SEQ ID NO: 312;
da) the CDR-H1 sequence comprises an amino acid sequence set forth in SEQ ID NO: 313, the CDR-H2 sequence comprises an amino acid sequence set forth in SEQ ID NO: 314, and the CDR-H3 sequence comprises an amino acid sequence set forth in SEQ ID NO: 315;
db) the CDR-H1 sequence comprises an amino acid sequence set forth in SEQ ID NO: 316, the CDR-H2 sequence comprises an amino acid sequence set forth in SEQ ID NO: 317, and the CDR-H3 sequence comprises an amino acid sequence set forth in SEQ ID NO: 318;
dc) the CDR-H1 sequence comprises an amino acid sequence set forth in SEQ ID NO: 319, the CDR-H2 sequence comprises an amino acid sequence set forth in SEQ ID NO: 320, and the CDR-H3 sequence comprises an amino acid sequence set forth in SEQ ID NO: 321;
dd) the CDR-H1 sequence comprises an amino acid sequence set forth in SEQ ID NO: 322, the CDR-H2 sequence comprises an amino acid sequence set forth in SEQ ID NO: 323, and the CDR-H3 sequence comprises an amino acid sequence set forth in SEQ ID NO: 324;
de) the CDR-H1 sequence comprises an amino acid sequence set forth in SEQ ID NO: 325, the CDR-H2 sequence comprises an amino acid sequence set forth in SEQ ID NO: 326, and the CDR-H3 sequence comprises an amino acid sequence set forth in SEQ ID NO: 327;
df) the CDR-H1 sequence comprises an amino acid sequence set forth in SEQ ID NO: 328, the CDR-H2 sequence comprises an amino acid sequence set forth in SEQ ID NO: 329, and the CDR-H3 sequence comprises an amino acid sequence set forth in SEQ ID NO: 330;
dg) the CDR-H1 sequence comprises an amino acid sequence set forth in SEQ ID NO: 331, the CDR-H2 sequence comprises an amino acid sequence set forth in SEQ ID NO: 332, and the CDR-H3 sequence comprises an amino acid sequence set forth in SEQ ID NO: 333;
dh) the CDR-H1 sequence comprises an amino acid sequence set forth in SEQ ID NO: 334, the CDR-H2 sequence comprises an amino acid sequence set forth in SEQ ID NO: 335, and the CDR-H3 sequence comprises an amino acid sequence set forth in SEQ ID NO: 336;
di) the CDR-H1 sequence comprises an amino acid sequence set forth in SEQ ID NO: 337, the CDR-H2 sequence comprises an amino acid sequence set forth in SEQ ID NO: 338, and the CDR-H3 sequence comprises an amino acid sequence set forth in SEQ ID NO: 339;
dj) the CDR-H1 sequence comprises an amino acid sequence set forth in SEQ ID NO: 340, the CDR-H2 sequence comprises an amino acid sequence set forth in SEQ ID NO: 341, and the CDR-H3 sequence comprises an amino acid sequence set forth in SEQ ID NO: 342;
dk) the CDR-H1 sequence comprises an amino acid sequence set forth in SEQ ID NO: 343, the CDR-H2 sequence comprises an amino acid sequence set forth in SEQ ID NO: 344, and the CDR-H3 sequence comprises an amino acid sequence set forth in SEQ ID NO: 345;
dl) the CDR-H1 sequence comprises an amino acid sequence set forth in SEQ ID NO: 346, the CDR-H2 sequence comprises an amino acid sequence set forth in SEQ ID NO: 347, and the CDR-H3 sequence comprises an amino acid sequence set forth in SEQ ID NO: 348;
dm) the CDR-H1 sequence comprises an amino acid sequence set forth in SEQ ID NO: 349, the CDR-H2 sequence comprises an amino acid sequence set forth in SEQ ID NO: 350, and the CDR-H3 sequence comprises an amino acid sequence set forth in SEQ ID NO: 351;
dn) the CDR-H1 sequence comprises an amino acid sequence set forth in SEQ ID NO: 352, the CDR-H2 sequence comprises an amino acid sequence set forth in SEQ ID NO: 353, and the CDR-H3 sequence comprises an amino acid sequence set forth in SEQ ID NO: 354;
do) the CDR-H1 sequence comprises an amino acid sequence set forth in SEQ ID NO: 355, the CDR-H2 sequence comprises an amino acid sequence set forth in SEQ ID NO: 356, and the CDR-H3 sequence comprises an amino acid sequence set forth in SEQ ID NO: 357;
dp) the CDR-H1 sequence comprises an amino acid sequence set forth in SEQ ID NO: 358, the CDR-H2 sequence comprises an amino acid sequence set forth in SEQ ID NO: 359, and the CDR-H3 sequence comprises an amino acid sequence set forth in SEQ ID NO: 360;
dq) the CDR-H1 sequence comprises an amino acid sequence set forth in SEQ ID NO: 361, the CDR-H2 sequence comprises an amino acid sequence set forth in SEQ ID NO: 362, and the CDR-H3 sequence comprises an amino acid sequence set forth in SEQ ID NO: 363;
dr) the CDR-H1 sequence comprises an amino acid sequence set forth in SEQ ID NO: 364, the CDR-H2 sequence comprises an amino acid sequence set forth in SEQ ID NO: 365, and the CDR-H3 sequence comprises an amino acid sequence set forth in SEQ ID NO: 366;
ds) the CDR-H1 sequence comprises an amino acid sequence set forth in SEQ ID NO: 367, the CDR-H2 sequence comprises an amino acid sequence set forth in SEQ ID NO: 368, and the CDR-H3 sequence comprises an amino acid sequence set forth in SEQ ID NO: 369;
dt) the CDR-H1 sequence comprises an amino acid sequence set forth in SEQ ID NO: 370, the CDR-H2 sequence comprises an amino acid sequence set forth in SEQ ID NO: 371, and the CDR-H3 sequence comprises an amino acid sequence set forth in SEQ ID NO: 372;
du) the CDR-H1 sequence comprises an amino acid sequence set forth in SEQ ID NO: 373, the CDR-H2 sequence comprises an amino acid sequence set forth in SEQ ID NO: 374, and the CDR-H3 sequence comprises an amino acid sequence set forth in SEQ ID NO: 375;
dv) the CDR-H1 sequence comprises an amino acid sequence set forth in SEQ ID NO: 376, the CDR-H2 sequence comprises an amino acid sequence set forth in SEQ ID NO: 377, and the CDR-H3 sequence comprises an amino acid sequence set forth in SEQ ID NO: 378;
dw) the CDR-H1 sequence comprises an amino acid sequence set forth in SEQ ID NO: 379, the CDR-H2 sequence comprises an amino acid sequence set forth in SEQ ID NO: 380, and the CDR-H3 sequence comprises an amino acid sequence set forth in SEQ ID NO: 381;
dx) the CDR-H1 sequence comprises an amino acid sequence set forth in SEQ ID NO: 382, the CDR-H2 sequence comprises an amino acid sequence set forth in SEQ ID NO: 383, and the CDR-H3 sequence comprises an amino acid sequence set forth in SEQ ID NO: 384;
dy) the CDR-H1 sequence comprises an amino acid sequence set forth in SEQ ID NO: 385, the CDR-H2 sequence comprises an amino acid sequence set forth in SEQ ID NO: 386, and the CDR-H3 sequence comprises an amino acid sequence set forth in SEQ ID NO: 387;
dz) the CDR-H1 sequence comprises an amino acid sequence set forth in SEQ ID NO: 388, the CDR-H2 sequence comprises an amino acid sequence set forth in SEQ ID NO: 389, and the CDR-H3 sequence comprises an amino acid sequence set forth in SEQ ID NO: 390;
ea) the CDR-H1 sequence comprises an amino acid sequence set forth in SEQ ID NO: 391, the CDR-H2 sequence comprises an amino acid sequence set forth in SEQ ID NO: 392, and the CDR-H3 sequence comprises an amino acid sequence set forth in SEQ ID NO: 393;
eb) the CDR-H1 sequence comprises an amino acid sequence set forth in SEQ ID NO: 394, the CDR-H2 sequence comprises an amino acid sequence set forth in SEQ ID NO: 395, and the CDR-H3 sequence comprises an amino acid sequence set forth in SEQ ID NO: 396;
ec) the CDR-H1 sequence comprises an amino acid sequence set forth in SEQ ID NO: 397, the CDR-H2 sequence comprises an amino acid sequence set forth in SEQ ID NO: 398, and the CDR-H3 sequence comprises an amino acid sequence set forth in SEQ ID NO: 399;
ed) the CDR-H1 sequence comprises an amino acid sequence set forth in SEQ ID NO: 400, the CDR-H2 sequence comprises an amino acid sequence set forth in SEQ ID NO: 401, and the CDR-H3 sequence comprises an amino acid sequence set forth in SEQ ID NO: 402;
ee) the CDR-H1 sequence comprises an amino acid sequence set forth in SEQ ID NO: 403, the CDR-H2 sequence comprises an amino acid sequence set forth in SEQ ID NO: 404, and the CDR-H3 sequence comprises an amino acid sequence set forth in SEQ ID NO: 405;
ef) the CDR-H1 sequence comprises an amino acid sequence set forth in SEQ ID NO: 406, the CDR-H2 sequence comprises an amino acid sequence set forth in SEQ ID NO: 407, and the CDR-H3 sequence comprises an amino acid sequence set forth in SEQ ID NO: 408;
eg) the CDR-H1 sequence comprises an amino acid sequence set forth in SEQ ID NO: 409, the CDR-H2 sequence comprises an amino acid sequence set forth in SEQ ID NO: 410, and the CDR-H3 sequence comprises an amino acid sequence set forth in SEQ ID NO: 411;
eh) the CDR-H1 sequence comprises an amino acid sequence set forth in SEQ ID NO: 412, the CDR-H2 sequence comprises an amino acid sequence set forth in SEQ ID NO: 413, and the CDR-H3 sequence comprises an amino acid sequence set forth in SEQ ID NO: 414;
ei) the CDR-H1 sequence comprises an amino acid sequence set forth in SEQ ID NO: 415, the CDR-H2 sequence comprises an amino acid sequence set forth in SEQ ID NO: 416, and the CDR-H3 sequence comprises an amino acid sequence set forth in SEQ ID NO: 417;
ej) the CDR-H1 sequence comprises an amino acid sequence set forth in SEQ ID NO: 418, the CDR-H2 sequence comprises an amino acid sequence set forth in SEQ ID NO: 419, and the CDR-H3 sequence comprises an amino acid sequence set forth in SEQ ID NO: 420;
ek) the CDR-H1 sequence comprises an amino acid sequence set forth in SEQ ID NO: 421, the CDR-H2 sequence comprises an amino acid sequence set forth in SEQ ID NO: 422, and the CDR-H3 sequence comprises an amino acid sequence set forth in SEQ ID NO: 423;
el) the CDR-H1 sequence comprises an amino acid sequence set forth in SEQ ID NO: 424, the CDR-H2 sequence comprises an amino acid sequence set forth in SEQ ID NO: 425, and the CDR-H3 sequence comprises an amino acid sequence set forth in SEQ ID NO: 426;
em) the CDR-H1 sequence comprises an amino acid sequence set forth in SEQ ID NO: 427, the CDR-H2 sequence comprises an amino acid sequence set forth in SEQ ID NO: 428, and the CDR-H3 sequence comprises an amino acid sequence set forth in SEQ ID NO: 429;
en) the CDR-H1 sequence comprises an amino acid sequence set forth in SEQ ID NO: 430, the CDR-H2 sequence comprises an amino acid sequence set forth in SEQ ID NO: 431, and the CDR-H3 sequence comprises an amino acid sequence set forth in SEQ ID NO: 432;
eo) the CDR-H1 sequence comprises an amino acid sequence set forth in SEQ ID NO: 433, the CDR-H2 sequence comprises an amino acid sequence set forth in SEQ ID NO: 434, and the CDR-H3 sequence comprises an amino acid sequence set forth in SEQ ID NO: 435;
ep) the CDR-H1 sequence comprises an amino acid sequence set forth in SEQ ID NO: 436, the CDR-H2 sequence comprises an amino acid sequence set forth in SEQ ID NO: 437, and the CDR-H3 sequence comprises an amino acid sequence set forth in SEQ ID NO: 438;
eq) the CDR-H1 sequence comprises an amino acid sequence set forth in SEQ ID NO: 439, the CDR-H2 sequence comprises an amino acid sequence set forth in SEQ ID NO: 440, and the CDR-H3 sequence comprises an amino acid sequence set forth in SEQ ID NO: 441;
er) the CDR-H1 sequence comprises an amino acid sequence set forth in SEQ ID NO: 442, the CDR-H2 sequence comprises an amino acid sequence set forth in SEQ ID NO: 443, and the CDR-H3 sequence comprises an amino acid sequence set forth in SEQ ID NO: 444;
es) the CDR-H1 sequence comprises an amino acid sequence set forth in SEQ ID NO: 445, the CDR-H2 sequence comprises an amino acid sequence set forth in SEQ ID NO: 446, and the CDR-H3 sequence comprises an amino acid sequence set forth in SEQ ID NO: 447;
et) the CDR-H1 sequence comprises an amino acid sequence set forth in SEQ ID NO: 448, the CDR-H2 sequence comprises an amino acid sequence set forth in SEQ ID NO: 449, and the CDR-H3 sequence comprises an amino acid sequence set forth in SEQ ID NO: 450; or
eu) the CDR-H1 sequence comprises an amino acid sequence set forth in SEQ ID NO: 451, the CDR-H2 sequence comprises an amino acid sequence set forth in SEQ ID NO: 452, and the CDR-H3 sequence comprises an amino acid sequence set forth in SEQ ID NO: 453.

In certain embodiments:
a) the ISVD domain comprises an amino acid sequence set forth in SEQ ID NO: 454;
b) the ISVD domain comprises an amino acid sequence set forth in SEQ ID NO: 455;
c) the ISVD domain comprises an amino acid sequence set forth in SEQ ID NO: 456;
d) the ISVD domain comprises an amino acid sequence set forth in SEQ ID NO: 457;
e) the ISVD domain comprises an amino acid sequence set forth in SEQ ID NO: 458;
f) the ISVD domain comprises an amino acid sequence set forth in SEQ ID NO: 459;
g) the ISVD domain comprises an amino acid sequence set forth in SEQ ID NO: 460;
h) the ISVD domain comprises an amino acid sequence set forth in SEQ ID NO: 461;
i) the ISVD domain comprises an amino acid sequence set forth in SEQ ID NO: 462;
j) the ISVD domain comprises an amino acid sequence set forth in SEQ ID NO: 463;
k) the ISVD domain comprises an amino acid sequence set forth in SEQ ID NO: 464;
l) the ISVD domain comprises an amino acid sequence set forth in SEQ ID NO: 465;
m) the ISVD domain comprises an amino acid sequence set forth in SEQ ID NO: 466;
n) the ISVD domain comprises an amino acid sequence set forth in SEQ ID NO: 467;
o) the ISVD domain comprises an amino acid sequence set forth in SEQ ID NO: 468;
p) the ISVD domain comprises an amino acid sequence set forth in SEQ ID NO: 469;
q) the ISVD domain comprises an amino acid sequence set forth in SEQ ID NO: 470;
r) the ISVD domain comprises an amino acid sequence set forth in SEQ ID NO: 471;
s) the ISVD domain comprises an amino acid sequence set forth in SEQ ID NO: 472;
t) the ISVD domain comprises an amino acid sequence set forth in SEQ ID NO: 473;
u) the ISVD domain comprises an amino acid sequence set forth in SEQ ID NO: 474;
v) the ISVD domain comprises an amino acid sequence set forth in SEQ ID NO: 475;
w) the ISVD domain comprises an amino acid sequence set forth in SEQ ID NO: 476;
x) the ISVD domain comprises an amino acid sequence set forth in SEQ ID NO: 477;
y) the ISVD domain comprises an amino acid sequence set forth in SEQ ID NO: 478;
z) the ISVD domain comprises an amino acid sequence set forth in SEQ ID NO: 479;
aa) the ISVD domain comprises an amino acid sequence set forth in SEQ ID NO: 480;
ab) the ISVD domain comprises an amino acid sequence set forth in SEQ ID NO: 481;
ac) the ISVD domain comprises an amino acid sequence set forth in SEQ ID NO: 482;
ad) the ISVD domain comprises an amino acid sequence set forth in SEQ ID NO: 483;
ae) the ISVD domain comprises an amino acid sequence set forth in SEQ ID NO: 484;
af) the ISVD domain comprises an amino acid sequence set forth in SEQ ID NO: 485;
ag) the ISVD domain comprises an amino acid sequence set forth in SEQ ID NO: 486;
ah) the ISVD domain comprises an amino acid sequence set forth in SEQ ID NO: 487;
ai) the ISVD domain comprises an amino acid sequence set forth in SEQ ID NO: 488;
aj) the ISVD domain comprises an amino acid sequence set forth in SEQ ID NO: 489;
ak) the ISVD domain comprises an amino acid sequence set forth in SEQ ID NO: 490;
al) the ISVD domain comprises an amino acid sequence set forth in SEQ ID NO: 491;
am) the ISVD domain comprises an amino acid sequence set forth in SEQ ID NO: 492;
an) the ISVD domain comprises an amino acid sequence set forth in SEQ ID NO: 493;
ao) the ISVD domain comprises an amino acid sequence set forth in SEQ ID NO: 494;
ap) the ISVD domain comprises an amino acid sequence set forth in SEQ ID NO: 495;
aq) the ISVD domain comprises an amino acid sequence set forth in SEQ ID NO: 496;
ar) the ISVD domain comprises an amino acid sequence set forth in SEQ ID NO: 497;
as) the ISVD domain comprises an amino acid sequence set forth in SEQ ID NO: 498;
at) the ISVD domain comprises an amino acid sequence set forth in SEQ ID NO: 499;
au) the ISVD domain comprises an amino acid sequence set forth in SEQ ID NO: 500;
av) the ISVD domain comprises an amino acid sequence set forth in SEQ ID NO: 501;
aw) the ISVD domain comprises an amino acid sequence set forth in SEQ ID NO: 502;
ax) the ISVD domain comprises an amino acid sequence set forth in SEQ ID NO: 503;
ay) the ISVD domain comprises an amino acid sequence set forth in SEQ ID NO: 504;
az) the ISVD domain comprises an amino acid sequence set forth in SEQ ID NO: 505;
ba) the ISVD domain comprises an amino acid sequence set forth in SEQ ID NO: 506;
bb) the ISVD domain comprises an amino acid sequence set forth in SEQ ID NO: 507;
bc) the ISVD domain comprises an amino acid sequence set forth in SEQ ID NO: 508;
bd) the ISVD domain comprises an amino acid sequence set forth in SEQ ID NO: 509;
be) the ISVD domain comprises an amino acid sequence set forth in SEQ ID NO: 510;
bf) the ISVD domain comprises an amino acid sequence set forth in SEQ ID NO: 511;
bg) the ISVD domain comprises an amino acid sequence set forth in SEQ ID NO: 512;
bh) the ISVD domain comprises an amino acid sequence set forth in SEQ ID NO: 513;
bi) the ISVD domain comprises an amino acid sequence set forth in SEQ ID NO: 514;
bj) the ISVD domain comprises an amino acid sequence set forth in SEQ ID NO: 515;
bk) the ISVD domain comprises an amino acid sequence set forth in SEQ ID NO: 516;
bl) the ISVD domain comprises an amino acid sequence set forth in SEQ ID NO: 517;
bm) the ISVD domain comprises an amino acid sequence set forth in SEQ ID NO: 518;
bn) the ISVD domain comprises an amino acid sequence set forth in SEQ ID NO: 519;
bo) the ISVD domain comprises an amino acid sequence set forth in SEQ ID NO: 520;
bp) the ISVD domain comprises an amino acid sequence set forth in SEQ ID NO: 521;
bq) the ISVD domain comprises an amino acid sequence set forth in SEQ ID NO: 522;
br) the ISVD domain comprises an amino acid sequence set forth in SEQ ID NO: 523;
bs) the ISVD domain comprises an amino acid sequence set forth in SEQ ID NO: 524;
bt) the ISVD domain comprises an amino acid sequence set forth in SEQ ID NO: 525;
bu) the ISVD domain comprises an amino acid sequence set forth in SEQ ID NO: 526;
by) the ISVD domain comprises an amino acid sequence set forth in SEQ ID NO: 527;
bw) the ISVD domain comprises an amino acid sequence set forth in SEQ ID NO: 528;
bx) the ISVD domain comprises an amino acid sequence set forth in SEQ ID NO: 529;
by) the ISVD domain comprises an amino acid sequence set forth in SEQ ID NO: 530;
bz) the ISVD domain comprises an amino acid sequence set forth in SEQ ID NO: 531;
ca) the ISVD domain comprises an amino acid sequence set forth in SEQ ID NO: 532;
cb) the ISVD domain comprises an amino acid sequence set forth in SEQ ID NO: 533;
cc) the ISVD domain comprises an amino acid sequence set forth in SEQ ID NO: 534;
cd) the ISVD domain comprises an amino acid sequence set forth in SEQ ID NO: 535;
ce) the ISVD domain comprises an amino acid sequence set forth in SEQ ID NO: 536;
cf) the ISVD domain comprises an amino acid sequence set forth in SEQ ID NO: 537;
cg) the ISVD domain comprises an amino acid sequence set forth in SEQ ID NO: 538;
ch) the ISVD domain comprises an amino acid sequence set forth in SEQ ID NO: 539;
ci) the ISVD domain comprises an amino acid sequence set forth in SEQ ID NO: 540;
cj) the ISVD domain comprises an amino acid sequence set forth in SEQ ID NO: 541;
ck) the ISVD domain comprises an amino acid sequence set forth in SEQ ID NO: 542;
cl) the ISVD domain comprises an amino acid sequence set forth in SEQ ID NO: 543;
cm) the ISVD domain comprises an amino acid sequence set forth in SEQ ID NO: 544;
cn) the ISVD domain comprises an amino acid sequence set forth in SEQ ID NO: 545;
co) the ISVD domain comprises an amino acid sequence set forth in SEQ ID NO: 546;
cp) the ISVD domain comprises an amino acid sequence set forth in SEQ ID NO: 547;
cq) the ISVD domain comprises an amino acid sequence set forth in SEQ ID NO: 548;
cr) the ISVD domain comprises an amino acid sequence set forth in SEQ ID NO: 549;
cs) the ISVD domain comprises an amino acid sequence set forth in SEQ ID NO: 550;
ct) the ISVD domain comprises an amino acid sequence set forth in SEQ ID NO: 551;
cu) the ISVD domain comprises an amino acid sequence set forth in SEQ ID NO: 552;
cv) the ISVD domain comprises an amino acid sequence set forth in SEQ ID NO: 553;
cw) the ISVD domain comprises an amino acid sequence set forth in SEQ ID NO: 554;
cx) the ISVD domain comprises an amino acid sequence set forth in SEQ ID NO: 555;
cy) the ISVD domain comprises an amino acid sequence set forth in SEQ ID NO: 556;
cz) the ISVD domain comprises an amino acid sequence set forth in SEQ ID NO: 557;
da) the ISVD domain comprises an amino acid sequence set forth in SEQ ID NO: 558;
db) the ISVD domain comprises an amino acid sequence set forth in SEQ ID NO: 559;
dc) the ISVD domain comprises an amino acid sequence set forth in SEQ ID NO: 560;
dd) the ISVD domain comprises an amino acid sequence set forth in SEQ ID NO: 561;
de) the ISVD domain comprises an amino acid sequence set forth in SEQ ID NO: 562;
df) the ISVD domain comprises an amino acid sequence set forth in SEQ ID NO: 563;
dg) the ISVD domain comprises an amino acid sequence set forth in SEQ ID NO: 564;
dh) the ISVD domain comprises an amino acid sequence set forth in SEQ ID NO: 565;
di) the ISVD domain comprises an amino acid sequence set forth in SEQ ID NO: 566;
dj) the ISVD domain comprises an amino acid sequence set forth in SEQ ID NO: 567;
dk) the ISVD domain comprises an amino acid sequence set forth in SEQ ID NO: 568;
dl) the ISVD domain comprises an amino acid sequence set forth in SEQ ID NO: 569;
dm) the ISVD domain comprises an amino acid sequence set forth in SEQ ID NO: 570;
dn) the ISVD domain comprises an amino acid sequence set forth in SEQ ID NO: 571;
do) the ISVD domain comprises an amino acid sequence set forth in SEQ ID NO: 572;
dp) the ISVD domain comprises an amino acid sequence set forth in SEQ ID NO: 573;
dq) the ISVD domain comprises an amino acid sequence set forth in SEQ ID NO: 574;
dr) the ISVD domain comprises an amino acid sequence set forth in SEQ ID NO: 575;
ds) the ISVD domain comprises an amino acid sequence set forth in SEQ ID NO: 576;
dt) the ISVD domain comprises an amino acid sequence set forth in SEQ ID NO: 577;
du) the ISVD domain comprises an amino acid sequence set forth in SEQ ID NO: 578;
dv) the ISVD domain comprises an amino acid sequence set forth in SEQ ID NO: 579;
dw) the ISVD domain comprises an amino acid sequence set forth in SEQ ID NO: 580;
dx) the ISVD domain comprises an amino acid sequence set forth in SEQ ID NO: 581;
dy) the ISVD domain comprises an amino acid sequence set forth in SEQ ID NO: 582;
dz) the ISVD domain comprises an amino acid sequence set forth in SEQ ID NO: 583;
ea) the ISVD domain comprises an amino acid sequence set forth in SEQ ID NO: 584;
eb) the ISVD domain comprises an amino acid sequence set forth in SEQ ID NO: 585;
ec) the ISVD domain comprises an amino acid sequence set forth in SEQ ID NO: 586;
ed) the ISVD domain comprises an amino acid sequence set forth in SEQ ID NO: 587;
ee) the ISVD domain comprises an amino acid sequence set forth in SEQ ID NO: 588;
ef) the ISVD domain comprises an amino acid sequence set forth in SEQ ID NO: 589;
eg) the ISVD domain comprises an amino acid sequence set forth in SEQ ID NO: 590;
eh) the ISVD domain comprises an amino acid sequence set forth in SEQ ID NO: 591;
ei) the ISVD domain comprises an amino acid sequence set forth in SEQ ID NO: 592;
ej) the ISVD domain comprises an amino acid sequence set forth in SEQ ID NO: 593;
ek) the ISVD domain comprises an amino acid sequence set forth in SEQ ID NO: 594;
el) the ISVD domain comprises an amino acid sequence set forth in SEQ ID NO: 595;
em) the ISVD domain comprises an amino acid sequence set forth in SEQ ID NO: 596;
en) the ISVD domain comprises an amino acid sequence set forth in SEQ ID NO: 597;
eo) the ISVD domain comprises an amino acid sequence set forth in SEQ ID NO: 598;
ep) the ISVD domain comprises an amino acid sequence set forth in SEQ ID NO: 599;
eq) the ISVD domain comprises an amino acid sequence set forth in SEQ ID NO: 600;
er) the ISVD domain comprises an amino acid sequence set forth in SEQ ID NO: 601;
es) the ISVD domain comprises an amino acid sequence set forth in SEQ ID NO: 602;
et) the ISVD domain comprises an amino acid sequence set forth in SEQ ID NO: 603; or
eu) the ISVD domain comprises an amino acid sequence set forth in SEQ ID NO: 604.

In certain embodiments, the antibody or antigen-binding fragment thereof is a chimeric or humanized antibody or antigen-binding fragment thereof.

In certain embodiments, the antibody or antigen-binding fragment thereof is a monoclonal antibody or antigen-binding fragment thereof.

In certain embodiments, the antibody or antigen-binding fragment thereof is a bispecific antibody.

In certain embodiments, the bispecific antibody comprises an antigen binding domain comprising binding affinity to a tumor associated antigen (TAA).

In certain embodiments, the ISVD is operatively linked to a CH1 domain and/or a CL domain.

In certain embodiments of the bispecific antibody, the ISVD with binding affinity to CD28 is operatively linked to a CH1 domain and the antigen binding domain with binding affinity to the TAA is operatively linked to a CL domain.

In certain embodiments of the bispecific antibody, the ISVD with binding affinity to CD28 is operatively linked to a CL domain and the antigen binding domain with binding affinity to the TAA is operatively linked to a CH1 domain.

In certain embodiments, the antibody or antigen-binding fragment thereof is operatively linked to an Fc region.

In certain embodiments, the Fc region is a human IgG1 Fc region.

In certain embodiments, the antibody or antigen-binding fragment thereof comprises an antagonistic antibody or antigen-binding fragment thereof.

In one aspect, the disclosure provides an isolated nucleic acid molecule encoding the antibody or antigen-binding fragment thereof of described above or the bispecific antibody described above.

In one aspect, the disclosure provides an expression vector comprising the nucleic acid molecule described above.

In one aspect, the disclosure provides a host cell comprising the expression vector described above.

In one aspect, the disclosure provides a method for inhibiting CD28 activity in a subject, comprising administering to a subject the antibody or antigen-binding fragment thereof described above, thereby inhibiting CD28 activity in the subject.

In one aspect, the disclosure provides a method for treating diseases associated with CD28 activity in a subject, comprising administering to a subject in need thereof the antibody or antigen-binding fragment thereof described above.

In certain embodiments, the disease is an autoimmune disease.

In certain embodiments, the disease is cancer.

The summary of the disclosure described above is non-limiting and other features and advantages of the disclosed antigen-binding proteins and methods will be apparent from the following brief description of the drawings, detailed description of the disclosure, and claims.

### BRIEF DESCRIPTION OF THE DRAWINGS

The foregoing and other features and advantages of the present invention will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1 shows the general workflow for CD28 x TAA bispecific screening.

FIG. 2 shows the sequence diversity of all CD28 binders identified in the primary screening.

FIGS. 3A-3B show the epitope binning of CD28 bispecific Fabs (bsFabs). Competition between anti-CD28 VHH was explored with SPR. FIG. 3A shows the experimental scheme of the competition between anti-CD28 VHH explored with SPR. FIG. 3B shows the competition data collected in a heatmap.

FIGS. 4A-4D show the evaluation of CD28 bsFabs and bivalent Fabs (bvFabs) in the IL-2 luciferase reporter assay with CD3 preactivation. FIG. 4A shows monovalent anti-CD28 x anti-foot-and-mouth disease virus (FMDV) bsFab with and without cross-linking.

FIG. 4B shows bivalent CD28 bvFab with and without cross-linking. FIG. 4C shows monovalent CD28xFMDV bsFab versus bivalent CD28 bvFab without cross-linking. FIG. 4D shows monovalent CD28xFMDV bsFab versus bivalent CD28 bvFab with cross-linking.

FIGS. 5A-5B show the evaluation of CD28 bsFabs and bvFabs in a T-cell activation assay. FIG. 5A shows the experimental setting. FIG. 5B shows IFNγ secretion on day 6 after treatment at 100 nM.

FIGS. 6A-6B show the evaluation of CD28 bvFabs in an MLR assay. FIG. 6A shows the experimental setting of the MLR assay. FIG. 6B shows IFNγ secretion on day 4 after treatment at 10 nM.

FIG. 7 displays a table containing full epitope binning results on the 48 CD28 VHHs.

FIG. 8 shows the competition of CD28 bvFabs with CD80 using flow cytometry.

FIG. 9 shows CD28 x TAA bsFabs simultaneously binding two cells expressing either CD28 or TAA.

FIGS. 10A-10F show the evaluation of CD28 bsFabs and bvFabs in the IL-2 luciferase reporter assay. FIG. 10A shows monovalent CD28 x FMDV bsFab with and without cross-linking in the absence of CD3 preactivation. FIG. 10B shows bivalent CD28 bvFab with and without cross-linking in the absence of CD3 preactivation. FIG. 10C shows monovalent CD28 x FMDV bsFab with and without cross-linking after CD3 preactivation. FIG. 10D shows bivalent CD28 bvFab with and without cross-linking after CD3 preactivation. FIG. 10E shows monovalent CD28 x FMDV bsFab versus bivalent CD28 bvFab without cross-linking after CD3 preactivation. FIG. 10F shows monovalent CD28 x FMDV bsFab versus bivalent CD28 bvFab with cross-linking after CD3 preactivation.

FIG. 11 shows the full epitope binning results of the 48 CD28 VHHs.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

Although any methods and materials similar or equivalent to those described herein can be used in the practice of the present disclosure, exemplary methods and materials are now described. All publications mentioned herein are incorporated herein by reference to describe in their entirety.

The term “about” or “approximately” means within about 20%, such as within about 10%, within about 5%, or within about 1% or less of a given value or range.

As used herein, the term “antibody” or “antigen-binding protein” refers to an immunoglobulin molecule that specifically binds to, or is immunologically reactive with an antigen or epitope, and includes both polyclonal and monoclonal antibodies, as well as functional antibody fragments thereof. The term “antibody” or “antigen-binding protein” includes immunoglobulin single variable domain (ISVD or ISV) antibodies (e.g., sdAb, sdFv, Nanobody®, VHH). The term “antibody” includes genetically engineered or otherwise modified forms of immunoglobulins, such as intrabodies, peptibodies, chimeric antibodies, fully human antibodies, humanized antibodies, meditope-enabled antibodies, heteroconjugate antibodies (e.g., multispecific antibodies, bispecific antibodies, diabodies, triabodies, tetrabodies, tandem di-scFv, tandem tri-scFv) and the like.

As used herein, the term “functional antibody fragment” refers to an antibody fragment having at least 80%, at least 85%, at least 90%, or at least 95% affinity as the antibody of interest from which the fragment is derived from.

The term “multispecific antibody” as used herein refers to bispecific, trispecific or multispecific antibodies, and antigen-binding fragments thereof. Multispecific antibodies may be specific for different epitopes of one target polypeptide or may contain antigen-binding domains specific for epitopes of more than one target polypeptide. A multispecific antibody can be a single multifunctional polypeptide, or it can be a multimeric complex of two or more polypeptides that are covalently or non-covalently associated with one another. The term “multispecific antibodies” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bispecific or a multispecific antibody with a second binding specificity. In certain exemplary embodiments, an antibody of the present disclosure is functionally linked to another antibody or antigen-binding fragment thereof to produce a bispecific antibody with a second binding specificity. In certain embodiments, the second binding specificity is to a tumor associated antigen (TAA).

As used herein, “monovalent” with reference to an antibody refers to antibodies that have a single antigen recognition site that is specific for a target antigen. Examples of monovalent antibodies include, a monovalent immunoglobulin single variable domain antibody (e.g., VHH) or a monovalent antibody fragment. Examples of monovalent antibody fragments include, but are not limited to, a Fab fragment, an Fv fragment, and a single-chain Fv fragment (scFv). Moreover, a multispecific antibody can have multiple antigen binding sites, each antigen binding site recognizing a different target antigen. Each antigen binding site would therefore be monovalent for the target antigen.

As used herein, “multivalent” with reference to an antibody refers to an antibody that has multiple (more than one) antigen recognition sites that are specific for a target antigen.

As used herein, the term “complementarity determining region” or “CDR” refers to sequences of amino acids within antibody variable regions, which confer antigen specificity and binding affinity. In general, there are three CDRs in each heavy chain variable region (CDR-H1, CDR-H2, CDR-H3). “Framework regions” or “FR” are known in the art to refer to the non-CDR portions of the variable regions of the heavy chain. In general, there are four FRs in each heavy chain variable region (FR-H1, FR-H2, FR-H3, and FR-H4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Pluckthun A, “Yet another numbering scheme for immunoglobulin variable domains: an automatic modeling and analysis tool,” J Mol Biol, 2001 Jun. 8; 309(3):657-70, (AHo numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

A “CDR” or “complementarity determining region,” or individual specified CDRs (e.g., “CDR-H1,” “CDR-H2,” “CDR-H3”), of a given antibody or region thereof, such as a variable region thereof, should be understood to encompass a (or the specific) complementarity determining region as defined by any of the known schemes. Likewise, an “FR” or “framework region,” or individual specified FRs (e.g., “FR-H1,” “FR-H2”) of a given antibody or region thereof, such as a variable region thereof, should be understood to encompass a (or the specific) framework region as defined by any of the known schemes. In some instances, the scheme for identification of a particular CDR or FR is specified, such as the CDR as defined by the IMGT, Kabat, Chothia, AbM, or Contact method. In other cases, the particular amino acid sequence of a CDR or FR is given. Unless otherwise specified, all particular CDR amino acid sequences mentioned in the disclosure are IMGT CDRs. However, alternative CDRs defined by other schemes are also encompassed by the present disclosure, such as those determined by abYsis Key Annotation (Website: abysis.org/abysis/sequence_input/key_annotation/key_annotation.cgi). Exemplary heavy chain CDR (HCDR) sequences of anti-CD28 VHH antibodies are recited in Table 1 below.

TABLE 1
Antibody HCDR amino acid sequences for each VHH antibody.

| | HCDR1- | HCDR1 | HCDR2- | HCDR2 | HCDR3- | HCDR3 |
|---|---|---|---|---|---|---|
| VHH ID | IMGT | SEQ ID NO: | IMGT | SEQ ID NO: | IMGT | SEQ ID NO: |
| 1 | GLTFRNYD | 1 | ASWSEEDT | 2 | AAGLTVN | 3 |
| | | | | | GRLLTRTY | |
| | | | | | EYDH | |
| 2 | GSISSIDH | 4 | INSGGRT | 5 | NVLLRDRS | 6 |
| | | | | | GSGRTY | |
| 3 | ERTAITYS | 7 | ISGRDGRT | 8 | ATSPLVST | 9 |
| | | | | | DQPDFYS | |
| 4 | GSIFRSVP | 10 | IFVDGST | 11 | FMNGD | 12 |
| 5 | ESRFSIKP | 13 | ITSPGMA | 14 | RDILSGS | 15 |
| 6 | GRLLSDNS | 16 | ITSGGGT | 17 | NWPRYGD | 18 |
| 7 | GRPFSSYA | 19 | IGGDGSTT | 20 | ALDFSLNR | 21 |
| | | | | | IVFGTRAD | |
| | | | | | Y | |
| 8 | GRTFSTW | 22 | IGGSGGSR | 23 | ATGPAAFG | 24 |
| | H | | | | SRKGTSYD | |
| | | | | | Y | |
| 9 | GRAFRINS | 25 | ISWSGRDT | 26 | AARVFFDS | 27 |
| | | | | | GSYAASEY | |
| | | | | | SN | |
| 10 | GDTFSNYA | 28 | ISWHGGR | 29 | AARLLGG | 30 |
| | | | A | | GWSGEEY | |
| | | | | | DY | |
| 11 | GRTFSGTA | 31 | IWWSRYA | 32 | AAGHRGY | 33 |
| | | | T | | SRFAEAYD | |
| | | | | | Y | |
| 12 | ERTFGIRT | 34 | IKWAGGN | 35 | AAAKVYY | 36 |
| | | | T | | YTPTMGP | |
| | | | | | GSYEF | |
| 13 | GRTGSHLD | 37 | ISRDGFRI | 38 | AADAAGF | 39 |
| | | | | | GSRFVSSY | |
| | | | | | DY | |
| 14 | GRTFDSDR | 40 | INWRGGG | 41 | VADIAAW | 42 |
| | FG | | A | | GARSAAS | |
| | | | | | YEY | |
| 15 | RAISSRWP | 43 | ISHGSIT | 44 | YAEDWDT | 45 |
| | | | | | RVQY | |
| 16 | GLTFSSYT | 46 | ASWSGGS | 47 | AAEKAPSR | 48 |
| | | | T | | TVAAYEY | |
| 17 | GRTSSGYA | 49 | IAWSAGST | 50 | AAGTRMP | 51 |
| | | | | | SRMTNAY | |
| | | | | | DY | |
| 18 | GRTSSGYA | 52 | ISWSGGST | 53 | AAGTRQV | 54 |
| | | | | | SQTTQAY | |
| | | | | | DY | |
| 19 | GRPFSSYI | 55 | INWSGDHT | 56 | AAKLSAGS | 57 |
| | | | | | STDTVLHN | |
| | | | | | NRWSWDS | |
| 20 | GGTYTTW | 58 | IRRTGGDP | 59 | AASPLWTS | 60 |
| | T | | | | SQDDYRH | |
| 21 | GAYLIVSD | 61 | IGRGGT | 62 | NIVDY | 63 |
| 22 | GFTFSSYW | 64 | MSPEGDM | 65 | VKGRTHG | 66 |
| | | | T | | SGLYGAR | |
| | | | | | DFES | |
| 23 | GYISSTHF | 67 | ITGSDLT | 68 | RLWGLGL | 69 |
| | | | | | GDGY | |
| 24 | GFTLSDY | 70 | IKAGDDTT | 71 | ARSPYGTY | 72 |
| | W | | | | RLDRRYDF | |
| 25 | GSSSSINA | 73 | ISRARGDS | 74 | YVAGDRS | 75 |
| | | | T | | LDFRSY | |
| 26 | GTISSTDF | 76 | ITGSDLT | 77 | RVWGLGY | 78 |
| | | | | | YY | |
| 27 | GRTFDYH | 79 | IGGSGGSR | 80 | ATGPAGY | 81 |
| | A | | | | GSRKSTSY | |
| | | | | | DY | |
| 28 | EEWFRINN | 82 | ITPSGST | 83 | RDISGGS | 84 |
| 29 | GRTLTDRT | 85 | IRWSDYRT | 86 | VAGHRLN | 87 |
| | LTDYA | | | | SRFAEAYN | |
| | | | | | Y | |
| 30 | ERPFSSYA | 88 | IGGDGSIT | 89 | ALDFSFNR | 90 |
| | | | | | IVLGSRAD | |
| | | | | | Y | |
| 31 | GRTFNA | 91 | IRWNGYM | 92 | AAGDRGS | 93 |
| | | | T | | SRFVAAY | |
| | | | | | DY | |
| 32 | GFTFDDYA | 94 | ISSSRATT | 95 | AAGTRMP | 96 |
| | | | | | SRMTNAY | |
| | | | | | DY | |
| 33 | RAISSRWP | 97 | ISHGSIT | 98 | YAAGQST | 99 |
| | | | | | APSASY | |
| 34 | VRTLTA | 100 | MRWSDGS | 101 | AADQVIFY | 102 |
| | | | T | | SRKPTDYD | |
| | | | | | Y | |
| 35 | GSISSFNA | 103 | ITGSGST | 104 | YADLSTY | 105 |
| | | | | | NAAWNGG | |
| | | | | | VYRNNY | |
| 36 | GLPFSTYF | 106 | IGGNGGSR | 107 | ATGPAAFG | 108 |
| | | | | | SRKTSSYD | |
| | | | | | Y | |
| 37 | ESRFSSKP | 109 | ITSPGMA | 110 | RDILSDS | 111 |
| 38 | GRSFSTYF | 112 | IGGNGGSR | 113 | ATGPRGFG | 114 |
| | | | | | SRKSTSYD | |
| | | | | | Y | |
| 39 | GFTLSDY | 115 | IASGGSDT | 116 | ARSPYGTY | 117 |
| | W | | | | RLDRRYD | |
| | | | | | T | |
| 40 | GYISSTHF | 118 | ITGSDLT | 119 | RLWGLGL | 120 |
| | | | | | GDGY | |
| 41 | GSIPSTHF | 121 | ITGSDLT | 122 | RVWGVGY | 123 |
| | | | | | DY | |
| 42 | GRPFSSYA | 124 | IGGDGSTT | 125 | ALDFSLNR | 126 |
| | | | | | IVFGTRAD | |
| | | | | | Y | |
| 43 | GRLISADS | 127 | ITSGGGT | 128 | HWPRYGD | 129 |
| 44 | GLTFSSYS | 130 | ISWSNGRT | 131 | AAEKAPSR | 132 |
| | | | | | TVAAYEY | |
| 45 | GSSSSINA | 133 | ISRARGDS | 134 | YVAGNRD | 135 |
| | | | T | | LDFRSY | |
| 46 | GSISSTDF | 136 | ITGSDLT | 137 | RVWGLGY | 138 |
| | | | | | AY | |
| 47 | GLTFSSYS | 139 | ISWSGGRT | 140 | AAERAPSR | 141 |
| | | | | | QVAAYEF | |
| 48 | GRTVSNY | 142 | VAWTGGR | 143 | AARLLGG | 144 |
| | A | | T | | GWSGEEY | |
| | | | | | DS | |
| 49 | RSISSRWP | 145 | ISHGSII | 146 | YAEDWDT | 147 |
| | | | | | RVQY | |
| 50 | GSIPSTHF | 148 | ITGSDLT | 149 | RVWGVGY | 150 |
| | | | | | DY | |
| 51 | ERTFSTYF | 151 | IGGNGGSR | 152 | ATGPRGFG | 153 |
| | | | | | SRKSTSYD | |
| | | | | | Y | |
| 52 | GRIFSTYS | 154 | IGGSGGSR | 155 | ATGPRGFG | 156 |
| | | | | | SRKTTSYD | |
| | | | | | Y | |
| 53 | GRTVSSYA | 157 | MSWTGGR | 158 | AGRLLGG | 159 |
| | | | T | | GWSGEEY | |
| | | | | | GY | |
| 54 | GRAFSTYF | 160 | IGGNGGSR | 161 | ETGPRGFG | 162 |
| | | | | | SRKNISYD | |
| | | | | | Y | |
| 55 | GLTFSSYS | 163 | ISWSGGRT | 164 | AAERAPSR | 165 |
| | | | | | QVAAYEY | |
| 56 | GFTFSTYS | 166 | ISWSNGRT | 167 | AAEKAPSR | 168 |
| | | | | | KVSAYEY | |
| 57 | ERTFSTYF | 169 | IGGNGGSR | 170 | ATGPRGFG | 171 |
| | | | | | SRKSTSYD | |
| | | | | | Y | |
| 58 | GLTFSSYS | 172 | ISWSGGRT | 173 | AAERAPSR | 174 |
| | | | | | QVAAYEF | |
| 59 | GSIPSTHF | 175 | ITGSDLT | 176 | RVWGVGY | 177 |
| | | | | | DY | |
| 60 | GRTTSTYF | 178 | IGGSGGSR | 179 | ATGPRGFG | 180 |
| | | | | | SRKSTSYD | |
| | | | | | Y | |
| 61 | GSIPSTHF | 181 | ITGSDLT | 182 | RVWGVGY | 183 |
| | | | | | DY | |
| 62 | GGTFSSYA | 184 | IAWHGGR | 185 | AARLLGG | 186 |
| | | | T | | GWSGEEY | |
| | | | | | EY | |
| 63 | GSSSSINA | 187 | ISRARGDS | 188 | YVAGDRS | 189 |
| | | | T | | LDFRSY | |
| 64 | GRSFSTYF | 190 | IGGNGGSR | 191 | ATGPRGFG | 192 |
| | | | | | SRKSTSYD | |
| | | | | | Y | |
| 65 | GRTVSTYF | 193 | IGGSGGSR | 194 | ATGPAGFG | 195 |
| | | | | | SRKSTSYD | |
| | | | | | Y | |
| 66 | GLTFSSYS | 196 | ISWSGGRT | 197 | AAERAPSR | 198 |
| | | | | | QVAAYEY | |
| 67 | GSIASTHF | 199 | LTESSLT | 200 | GVWGLGF | 201 |
| | | | | | AY | |
| 68 | ERTFDTYT | 202 | IGGSGGSR | 203 | ATGPAGFG | 204 |
| | | | | | SRKTTSYD | |
| | | | | | Y | |
| 69 | GLTFSSYS | 205 | ISWSAGRT | 206 | AAERAPSR | 207 |
| | | | | | QVAAYEY | |
| 70 | GLTFSSYS | 208 | ISWSAGRT | 209 | AAERAPSR | 210 |
| | | | | | QVAAYEY | |
| 71 | RSISSRWP | 211 | ISHGSII | 212 | YAEDWDT | 213 |
| | | | | | RVQY | |
| 72 | GRPFSSYA | 214 | IGGDGSTT | 215 | ALDFSLNR | 216 |
| | | | | | IVFGTRAD | |
| | | | | | Y | |
| 73 | GLTFSSYS | 217 | ISWSGGRT | 218 | AAERAPSR | 219 |
| | | | | | QVAAYEF | |
| 74 | GDRFSIKP | 220 | ITSPGTA | 221 | RDILSDS | 222 |
| 75 | GRTFSNYD | 223 | CSWGGEN | 224 | VAGLTVN | 225 |
| | | | TA | | GRLLTRTY | |
| | | | | | EYDN | |
| 76 | GRTFSSTA | 226 | IRWSDYRT | 227 | VAGHRLN | 228 |
| | | | | | SRFAEAYD | |
| | | | | | Y | |
| 77 | GRAFSTYH | 229 | IGGSGGSR | 230 | ATGPAGFG | 231 |
| | | | | | SRRSTSYD | |
| | | | | | Y | |
| 78 | GSISSTHF | 232 | ITESSLT | 233 | GVWGLGY | 234 |
| | | | | | AY | |
| 79 | GRTTSTYF | 235 | IGGSGGSR | 236 | ATGPRGFG | 237 |
| | | | | | SRKSTSYD | |
| | | | | | Y | |
| 80 | GSRFSTKP | 238 | ITTPGMA | 239 | RDILSDD | 240 |
| 81 | GRSFSTYF | 241 | IGGNGGSR | 242 | ATGPRGFG | 243 |
| | | | | | SRKSTSYD | |
| | | | | | Y | |
| 82 | GRTFSSTA | 244 | IRWSDYRT | 245 | VAGHRLN | 246 |
| | | | | | SRFAEAYD | |
| | | | | | Y | |
| 83 | GLPFSTYF | 247 | IGGNGGSR | 248 | ATGPAAFG | 249 |
| | | | | | SRKTSSYD | |
| | | | | | Y | |
| 84 | GRSFSTDF | 250 | IGGNGGSR | 251 | ATGPRGFG | 252 |
| | | | | | SRKSTSYD | |
| | | | | | Y | |
| 85 | GRLISANS | 253 | ITSGGGT | 254 | NWPRYGD | 255 |
| 86 | GLTFSSYS | 256 | ISWSAGRT | 257 | AAERAPSR | 258 |
| | | | | | RVDAYEY | |
| 87 | GRSFSTYF | 259 | IGGNGGSR | 260 | ATGPRGFG | 261 |
| | | | | | SRKSTSYD | |
| | | | | | Y | |
| 88 | GLTFSSYS | 262 | ISWSGGRT | 263 | AAERAPSR | 264 |
| | | | | | QVAAYEF | |
| 89 | GLTFSSYS | 265 | ISWSYGST | 266 | AAEKAPSR | 267 |
| | | | | | RVAAYEY | |
| 90 | GRSFSTYF | 268 | IGGNGGSR | 269 | ATGPRGFG | 270 |
| | | | | | SRKSTSYD | |
| | | | | | Y | |
| 91 | GYISSTHF | 271 | ITGSDLT | 272 | RLWGLGL | 273 |
| | | | | | GDGY | |
| 92 | GRTFSSTA | 274 | IRWSDYRT | 275 | VAGHGLN | 276 |
| | | | | | SRFAEAYD | |
| | | | | | Y | |
| 93 | GLTFSSYS | 277 | ISWSAGRT | 278 | AAERAPSR | 279 |
| | | | | | RVDAYEY | |
| 94 | GLTFSSYS | 280 | ISWSAGRT | 281 | AAERAPSR | 282 |
| | | | | | QVAAYEY | |
| 95 | GGTFSTYF | 283 | IGGSGGSR | 284 | ATGPRGFG | 285 |
| | | | | | SRKSTSYD | |
| | | | | | Y | |
| 96 | GRPFSSYI | 286 | INWSGDHT | 287 | AAKLSAGS | 288 |
| | | | | | STDTVLHN | |
| | | | | | NRWSWDY | |
| 97 | GLPFSTYF | 289 | IGGNGGSR | 290 | ATGPAAFG | 291 |
| | | | | | SRKTSSYD | |
| | | | | | Y | |
| 98 | ERTAFTYS | 292 | ISGRDGRT | 293 | ATSPLVST | 294 |
| | | | | | DQPDFYS | |
| 99 | GLTFSSYT | 295 | ASWSGGN | 296 | AAEKAPSR | 297 |
| | | | T | | TVAAYEY | |
| 100 | GRSFITYF | 298 | IGGNGGSR | 299 | ATGPRGFG | 300 |
| | | | | | SRKSTSYD | |
| | | | | | Y | |
| 101 | GRSFITYF | 301 | IGGNGGSR | 302 | ATGPRGFG | 303 |
| | | | | | SRKSNSYD | |
| | | | | | Y | |
| 102 | GRSFITYF | 304 | IGGNGGSR | 305 | ATGPRGFG | 306 |
| | | | | | SRKSTSYD | |
| | | | | | Y | |
| 103 | GRSFITYF | 307 | IGGNGGSR | 308 | ATGPRGFG | 309 |
| | | | | | SRKSTSYD | |
| | | | | | Y | |
| 104 | GSITSFNA | 310 | ITGSGST | 311 | YADLSTY | 312 |
| | | | | | NAEWNGG | |
| | | | | | AYRNNY | |
| 105 | GSSSSINA | 313 | ISRARGDS | 314 | YVAGNRD | 315 |
| | | | T | | LDFRSY | |
| 106 | GLTFSSYS | 316 | ISWSGGRT | 317 | AAERAPSR | 318 |
| | | | | | QVAAYEY | |
| 107 | GPTFGTYA | 319 | MSWSNGR | 320 | AAEKAPSR | 321 |
| | | | T | | KVSAYEY | |
| 108 | GRTFSSTA | 322 | IRWSDYRT | 323 | VAGHRLN | 324 |
| | | | | | SRFAEAYD | |
| | | | | | Y | |
| 109 | GRSFISYF | 325 | IGGNGGSR | 326 | ATGPRGFS | 327 |
| | | | | | SRKSTSYD | |
| | | | | | Y | |
| 110 | GRTFSTW | 328 | IGGSGGSR | 329 | ATGPAAFG | 330 |
| | H | | | | SRKSTSYD | |
| | | | | | Y | |
| 111 | ERTFDTYT | 331 | IGGSGGSR | 332 | ATGPAGFG | 333 |
| | | | | | SRKTTSYD | |
| | | | | | Y | |
| 112 | GRTSSGYA | 334 | IAWSAGST | 335 | AAGTRMP | 336 |
| | | | | | SRMTNAY | |
| | | | | | DY | |
| 113 | GRTFSSTA | 337 | IRWSDYRT | 338 | VAGHRLN | 339 |
| | | | | | SRFAEAYD | |
| | | | | | Y | |
| 114 | GRTVSTYF | 340 | IGGSGGSR | 341 | ATGPAGFG | 342 |
| | | | | | SRKTTSYD | |
| | | | | | Y | |
| 115 | GRTFSSTA | 343 | IRWSDYRT | 344 | VAGHRLN | 345 |
| | | | | | SRFAEAYD | |
| | | | | | Y | |
| 116 | GRTFSSTA | 346 | IRWSDYRT | 347 | VAGHRLIT | 348 |
| | | | | | RITEAYDY | |
| 117 | GRLISADS | 349 | ITSGGGT | 350 | NCPRYGD | 351 |
| 118 | GLTFSSYT | 352 | ASWSGGS | 353 | AAEKAPSQ | 354 |
| | | | T | | TVAAYEY | |
| 119 | GRLISANS | 355 | ITSGGGT | 356 | NWPRYGD | 357 |
| 120 | GRAFRINS | 358 | ISWSGRDT | 359 | AARVFFDS | 360 |
| | | | | | GSYAASEY | |
| | | | | | SN | |
| 121 | GRTTSTYF | 361 | IGGSGGSR | 362 | ATGPRGFG | 363 |
| | | | | | SRRSTSYD | |
| | | | | | Y | |
| 122 | GSSSSINA | 364 | ISRARGDS | 365 | YVAGNRD | 366 |
| | | | T | | LDFRSY | |
| 123 | ERTFSTYF | 367 | IGGNGGSR | 368 | ATGPRGFG | 369 |
| | | | | | SRKSTSYD | |
| | | | | | Y | |
| 124 | GRTSSTYF | 370 | IGGSGGSR | 371 | ATGPRGFG | 372 |
| | | | | | SRKSTSYD | |
| | | | | | Y | |
| 125 | GRPFSSYA | 373 | IGGDGSTT | 374 | ALDFSLNR | 375 |
| | | | | | IVFGTRAD | |
| | | | | | Y | |
| 126 | ATISSTDF | 376 | ITSSDLT | 377 | RVWGLGY | 378 |
| | | | | | YY | |
| 127 | GRTFSMY | 379 | IGGNGGSR | 380 | ATGPRGFG | 381 |
| | N | | | | SQKSNSYD | |
| | | | | | Y | |
| 128 | GRTVSTYF | 382 | IGGSGGSR | 383 | ATGPAGFG | 384 |
| | | | | | SRKTTSYD | |
| | | | | | Y | |
| 129 | FSSSY | 385 | IGGNGGSR | 386 | ATGPRGFG | 387 |
| | | | | | SRKSTSYD | |
| | | | | | Y | |
| 130 | GRAFSTYF | 388 | IGGSGGSR | 389 | ATGPRGFG | 390 |
| | | | | | SRKTTSYD | |
| | | | | | Y | |
| 131 | GRTFSSYA | 391 | ISWTGGRT | 392 | TARLLGG | 393 |
| | | | | | GWSGEEY | |
| | | | | | DY | |
| 132 | GRSFITYF | 394 | IGGNGGSR | 395 | ATGPRGFG | 396 |
| | | | | | SRKSTSYD | |
| | | | | | Y | |
| 133 | GRTASSYA | 397 | MPWTGGR | 398 | AARLLGG | 399 |
| | | | T | | GWSGEEY | |
| | | | | | DY | |
| 134 | GRSFSTYF | 400 | IGGNGGSR | 401 | ATGPRGFG | 402 |
| | | | | | SRKSTSYD | |
| | | | | | Y | |
| 135 | GLTFSSYS | 403 | ISWSGGRT | 404 | AAERAPSR | 405 |
| | | | | | QVAAYEF | |
| 136 | GSISSTHF | 406 | ITESSLT | 407 | GVWGLGY | 408 |
| | | | | | AY | |
| 137 | GSIASTHF | 409 | LTESSLT | 410 | GVWGLGF | 411 |
| | | | | | AY | |
| 138 | GSIFSTNI | 412 | ITGSDLT | 413 | RVWGLGY | 414 |
| | | | | | YY | |
| 139 | GGTFSTYF | 415 | IGGSGGSR | 416 | ATGPRGFG | 417 |
| | | | | | SRKSTSYD | |
| | | | | | Y | |
| 140 | GSISSTHF | 418 | ITTSSAT | 419 | GVWGVGY | 420 |
| | | | | | AY | |
| 141 | GYISSTHF | 421 | ITGSDLT | 422 | RLWGLGL | 423 |
| | | | | | GYGY | |
| 142 | GRPFSSYA | 424 | IGGDGSIT | 425 | ALDFSFNR | 426 |
| | | | | | IVLGTRAD | |
| | | | | | Y | |
| 143 | ERTFSTYF | 427 | IGGNGGSR | 428 | ATGPRGFG | 429 |
| | | | | | SRKSTSYD | |
| | | | | | Y | |
| 144 | GRTFSNYD | 430 | CGWKAED | 431 | AAGLTVN | 432 |
| | | | T | | GRLLTRTY | |
| | | | | | EYDI | |
| 145 | GRTVSTYF | 433 | IGGSGGSR | 434 | ATGPAGFG | 435 |
| | | | | | SRKSTSYD | |
| | | | | | Y | |
| 146 | GRSFSTYF | 436 | IGGNGGSR | 437 | ATGPRGFG | 438 |
| | | | | | SRKSNSYD | |
| | | | | | Y | |
| 147 | GRSFSTYF | 439 | IGGNGGSR | 440 | ATGPRGFG | 441 |
| | | | | | SRKSTSYD | |
| | | | | | Y | |
| 148 | GGTYTTYT | 442 | IRRTGGDP | 443 | AGSPLWTS | 444 |
| | | | | | SQDDYRH | |
| 149 | ERTFSTYF | 445 | IGGNGGSR | 446 | ATGPRGFG | 447 |
| | | | | | SRKSTSYD | |
| | | | | | Y | |
| 150 | GSIPSTHF | 448 | ITGSDVT | 449 | RVWGVGY | 450 |
| | | | | | DY | |
| 151 | GSIPSTHF | 451 | ITGSDLT | 452 | RVWGVGY | 453 |
| | | | | | DY | |

“Humanized” forms of non-human antibodies are chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, llama, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. A humanized sequence can be identified by its primary sequence and does not necessarily denote the process by which the antibody was created.

As used herein, the term “specifically binds,” “specifically binding,” “binding specificity” or “specifically recognized” refers that an antigen binding protein or antigen-binding fragment thereof that exhibits appreciable affinity for an antigen (e.g., a CD28 antigen) and does not exhibit significant cross reactivity to a target that is not a CD28 protein. As used herein, the term “affinity” refers to the strength of the interaction between an antigen binding protein or antigen-binding fragment thereof antigen binding site and the epitope to which it binds. In certain exemplary embodiments, affinity is measured by surface plasmon resonance (SPR), e.g., in a Biacore instrument. As readily understood by those skilled in the art, an antigen binding protein affinity may be reported as a dissociation constant (KD) in molarity (M).

Specific binding can be determined according to any art-recognized means for determining such binding. In some embodiments, specific binding is determined by competitive binding assays (e.g., ELISA) or Biacore assays. In certain embodiments, the assay is conducted at about 20° C., 25° C., 30° C., or 37° C.

The term “agonist” as used herein in reference to an antibody means that upon binding to the target protein expressed on the surface of a cell the antibody stimulates or activates signaling through the target protein.

The term “antagonist” as used herein in reference to an antibody means that upon binding to the target protein expressed on the surface of a cell the antibody inhibits signaling through the target protein.

### Immunoglobulin Single Variable Domain (ISVD)

The term “immunoglobulin single variable domain” (ISV or ISVD), interchangeably used with “single variable domain”, defines immunoglobulin molecules wherein the antigen binding site is present on, and formed by, a single immunoglobulin domain. This sets immunoglobulin single variable domains apart from “conventional” immunoglobulins (e.g. monoclonal antibodies) or their fragments (such as Fab, Fab′, F(ab′)₂, scFv, di-scFv), wherein two immunoglobulin domains, in particular two variable domains, interact to form an antigen binding site. Typically, in conventional immunoglobulins, a heavy chain variable domain (V_{H}) and a light chain variable domain (V_{L}) interact to form an antigen binding site. In this case, the complementarity determining regions (CDRs) of both V_{H} and V_{L} will contribute to the antigen binding site, i.e. a total of 6 CDRs will be involved in antigen binding site formation.

In view of the above definition, the antigen-binding domain of a conventional 4-chain antibody or of a Fab fragment, a F(ab′)₂ fragment, an Fv fragment such as a disulfide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e., by a V_{H}-V_{L} pair of immunoglobulin domains, which jointly bind to an epitope of the respective antigen.

In contrast, immunoglobulin single variable domains are capable of specifically binding to an epitope of the antigen without pairing with an additional immunoglobulin variable domain. The binding site of an immunoglobulin single variable domain is formed by a single V_{H}, a single VHH or single V_{L} domain.

As such, the single variable domain may be a light chain variable domain sequence (e.g., a V_{L}-sequence) or a suitable fragment thereof; or a heavy chain variable domain sequence (e.g., a V_{H}-sequence or V_{HH} sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

An immunoglobulin single variable domain (ISV) can for example be a heavy chain ISV, such as a V_{H}, V_{HH}, including a camelized V_{H} or humanized V_{HH}. In one embodiment, it is a V_{HH}, including a camelized V_{H} or humanized V_{HH}. Heavy chain ISVs can be derived from a conventional four-chain antibody or from a heavy chain antibody.

For example, the immunoglobulin single variable domain may be a single domain antibody (or an amino acid sequence that is suitable for use as a single domain antibody), a “dAb” or dAb (or an amino acid sequence that is suitable for use as a dAb) or a Nanobody® ISV (as defined herein, and including but not limited to a VHH); other single variable domains, or any suitable fragment of any one thereof.

In particular, the immunoglobulin single variable domain may be a Nanobody® ISV (such as a V_{HH}, including a humanized V_{HH} or camelized V_{H}) or a suitable fragment thereof. [Note: Nanobody® and Nanobodies® are registered trademarks of Ablynx N.V.]

“VHH domains”, also known as VHHS, VHH antibody fragments, and V_{HH} antibodies, have originally been described as the antigen binding immunoglobulin variable domain of “heavy chain antibodies” (i.e., of “antibodies devoid of light chains”; Hamers-Casterman et al. Nature 363: 446-448, 1993). The term “VHH domain” has been chosen in order to distinguish these variable domains from the heavy chain variable domains that are present in conventional 4-chain antibodies (which are referred to herein as “V_{H} domains”) and from the light chain variable domains that are present in conventional 4-chain antibodies (which are referred to herein as “V_{L} domains”). For a further description of V_{HH}'s, reference is made to the review article by Muyldermans (Reviews in Molecular Biotechnology 74: 277-302, 2001).

The generation of immunoglobulin sequences, such as VHHs, has been described extensively in various publications, among which WO 94/04678, Hamers-Casterman et al. 1993 and Muyldermans et al. 2001 (Reviews in Molecular Biotechnology 74: 277-302, 2001). In these methods, camelids are immunized with the target antigen in order to induce an immune response against said target antigen. The repertoire of VHHs obtained from said immunization is further screened for VHHs that bind the target antigen.

In these instances, the generation of antibodies requires purified antigen for immunization and/or screening. Antigens can be purified from natural sources, or in the course of recombinant production. Immunization and/or screening for immunoglobulin sequences can be performed using peptide fragments of such antigens.

Immunoglobulin sequences of different origin, comprising mouse, rat, rabbit, donkey, human and camelid immunoglobulin sequences can be sequenced in the method described herein. Also, fully human, humanized or chimeric sequences can be sequenced in the method described herein. For example, camelid immunoglobulin sequences and humanized camelid immunoglobulin sequences, or camelized domain antibodies, e.g. camelized dAb as described by Ward et al (see for example WO 94/04678 and Riechmann, Febs Lett., 339:285-290, 1994 and Prot. Eng., 9:531-537, 1996) can be sequenced in the method described herein. Moreover, the ISVs are fused forming a multivalent and/or multispecific construct (for multivalent and multispecific polypeptides containing one or more VHH domains and their preparation, reference is also made to Conrath et al., J. Biol. Chem., Vol. 276, 10. 7346-7350, 2001, as well as to for example WO 96/34103 and WO 99/23221).

A “humanized VHH” comprises an amino acid sequence that corresponds to the amino acid sequence of a naturally occurring VHH domain, but that has been “humanized”, i.e. by replacing one or more amino acid residues in the amino acid sequence of said naturally occurring VHH sequence (and in particular in the framework sequences) by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the prior art (e.g. WO 2008/020079). Again, it should be noted that such humanized VHHs can be obtained in any suitable manner known per se and thus are not strictly limited to polypeptides that have been obtained using a polypeptide that comprises a naturally occurring VHH domain as a starting material.

A “camelized V_{H}” comprises an amino acid sequence that corresponds to the amino acid sequence of a naturally occurring V_{H} domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a VHH domain of a (camelid) heavy chain antibody. This can be performed in a manner known per se, which will be clear to the skilled person, for example on the basis of the description in the prior art (e.g. Davies and Riechman (1994 and 1996), supra). Such “camelizing” substitutions are inserted at amino acid positions that form and/or are present at the V_{H}-V_{L} interface, and/or at the so-called Camelidae hallmark residues, as defined herein (see for example WO 94/04678 and Davies and Riechmann (1994 and 1996), supra). In one embodiment, the V_{H} sequence that is used as a starting material or starting point for generating or designing the camelized V_{H} is a V_{H} sequence from a mammal, such as the V_{H} sequence of a human being, such as a V_{H}3 sequence. However, it should be noted that such camelized V_{H} can be obtained in any suitable manner known per se and thus are not strictly limited to polypeptides that have been obtained using a polypeptide that comprises a naturally occurring V_{H} domain as a starting material.

The structure of an immunoglobulin single variable domain sequence can be considered to be comprised of four framework regions (“FRs”), which are referred to in the art and herein as “Framework region 1” (“FR1”); as “Framework region 2” (“FR2”); as “Framework region 3” (“FR3”); and as “Framework region 4” (“FR4”), respectively; which framework regions are interrupted by three complementary determining regions (“CDRs”), which are referred to in the art and herein as “Complementarity Determining Region 1” (“CDR1”); as “Complementarity Determining Region 2” (“CDR2”); and as “Complementarity Determining Region 3” (“CDR3”), respectively.

In such an immunoglobulin sequence, the framework sequences may be any suitable framework sequences, and examples of suitable framework sequences will be clear to the skilled person, for example on the basis the standard handbooks and the further disclosure and prior art mentioned herein.

The framework sequences are (a suitable combination of) immunoglobulin framework sequences or framework sequences that have been derived from immunoglobulin framework sequences (for example, by humanization or camelization). For example, the framework sequences may be framework sequences derived from a light chain variable domain (e.g. a V_{L}-sequence) and/or from a heavy chain variable domain (e.g. a V_{H}-sequence or VHH sequence). In one particular aspect, the framework sequences are either framework sequences that have been derived from a VHH-sequence (in which said framework sequences may optionally have been partially or fully humanized) or are conventional V_{H} sequences that have been camelized (as defined herein).

In particular, the framework sequences present in the ISV sequence used in the methods described herein may contain one or more of hallmark residues (as defined herein), such that the ISV sequence is a Nanobody® ISV, such as e.g. a V_{HH}, including a humanized V_{HH} or camelized V_{H}. Non-limiting examples of (suitable combinations of) such framework sequences will become clear from the further disclosure herein.

The total number of amino acid residues in a V_{H} domain and a V_{HH} domain will usually be in the range of from 110 to 120, often between 112 and 115. It should however be noted that smaller and longer sequences may also be suitable for the purposes described herein.

However, it should be noted that the ISVs comprised in the multivalent ISV polypeptide that is sequenced in the present method is not limited as to the origin of the ISV sequence (or of the nucleotide sequence used to express it), nor as to the way that the ISV sequence or nucleotide sequence is (or has been) generated or obtained. Thus, the ISV sequences may be naturally occurring sequences (from any suitable species) or synthetic or semi-synthetic sequences. In a specific but non-limiting aspect, the ISV sequence is a naturally occurring sequence (from any suitable species) or a synthetic or semi-synthetic sequence, including but not limited to “humanized” (as defined herein) immunoglobulin sequences (such as partially or fully humanized mouse or rabbit immunoglobulin sequences, and in particular partially or fully humanized V_{HH} sequences), “camelized” (as defined herein) immunoglobulin sequences (and in particular camelized V_{H} sequences), as well as ISVs that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing.

Similarly, nucleotide sequences may be naturally occurring nucleotide sequences or synthetic or semi-synthetic sequences, and may for example be sequences that are isolated by PCR from a suitable naturally occurring template (e.g. DNA or RNA isolated from a cell), nucleotide sequences that have been isolated from a library (and in particular, an expression library), nucleotide sequences that have been prepared by introducing mutations into a naturally occurring nucleotide sequence (using any suitable technique known per se, such as mismatch PCR), nucleotide sequence that have been prepared by PCR using overlapping primers, or nucleotide sequences that have been prepared using techniques for DNA synthesis known per se.

Generally, Nanobody® ISVs (in particular VHH sequences, including (partially) humanized VHH sequences and camelized V_{H} sequences) can be characterized by the presence of one or more “Hallmark residues” (as described herein) in one or more of the framework sequences (again as further described herein). Thus, generally, a Nanobody® ISV can be defined as an immunoglobulin sequence with the (general) structure
FR1-CDR1-FR2-CDR2-FR3-CDR3-FR4
in which FR1 to FR4 refer to framework regions 1 to 4, respectively, and in which CDR1 to CDR3 refer to the complementarity determining regions 1 to 3, respectively, and in which one or more of the Hallmark residues are as further defined herein.

In particular, a Nanobody® ISV can be an immunoglobulin sequence with the (general) structure
FR1-CDR1-FR2-CDR2-FR3-CDR3-FR4
in which FR1 to FR4 refer to framework regions 1 to 4, respectively, and in which CDR1 to CDR3 refer to the complementarity determining regions 1 to 3, respectively, and in which the framework sequences are as further defined herein.

More in particular, a Nanobody® ISV can be an immunoglobulin sequence with the (general) structure
FR1-CDR1-FR2-CDR2-FR3-CDR3-FR4
in which FR1 to FR4 refer to framework regions 1 to 4, respectively, and in which CDR1 to CDR3 refer to the complementarity determining regions 1 to 3, respectively, and in which:
one or more of the amino acid residues at positions 11, 37, 44, 45, 47, 83, 84, 103, 104 and 108 according to the Kabat numbering are chosen from the Hallmark residues mentioned in Table A below.

TABLE A
Hallmark Residues in Nanobody ® ISVs

| Position | Human V_{H}3 | Hallmark Residues |
|---|---|---|
| 11 | L, V; | L, S, V, M, W, F, T, Q, E, A, R, |
| | predominantly L | G, K, Y, N, P, I; preferably L |
| 37 | V, I, F; | F⁽¹⁾, Y, V, L, A, H, S, I, W, C, N, |
| | usually V | G, D, T, P, preferably F⁽¹⁾ or Y |
| 44⁽⁸⁾ | G | E⁽³⁾, Q⁽³⁾, G⁽²⁾, D, A, K, R, L, |
| | | P, S, V, H, T, N, W, M, I; |
| | | preferably G⁽²⁾, E⁽³⁾ or Q⁽³⁾; most |
| | | preferably G⁽²⁾ or Q⁽³⁾. |
| 45⁽⁸⁾ | L | L⁽²⁾, R⁽³⁾, P, H, F, G, Q, S, E, T, |
| | | Y, C, I, D, V; preferably L⁽²⁾ or R⁽³⁾ |
| 47⁽⁸⁾ | W, Y | F⁽¹⁾, L⁽¹⁾ or W⁽²⁾ G, I, S, A, V, M, |
| | | R, Y, E, P, T, C, H, K, Q, N, D; |
| | | preferably W⁽²⁾, L⁽¹⁾ or F⁽¹⁾ |
| 83 | R or K; | R, K⁽⁵⁾, T, E⁽⁵⁾, Q, N, S, I, V, G, |
| | usually R | M, L, A, D, Y, H; preferably K or |
| | | R; most preferably K |
| 84 | A, T, D; | P⁽⁵⁾, S, H, L, A, V, I, T, F, D, |
| | predominantly A | R, Y, N, Q, G, E; preferably P |
| 103 | W | W⁽⁴⁾, R⁽⁶⁾, G, S, K, A, M, Y, L, |
| | | F, T, N, V, Q, P⁽⁶⁾, E, C; |
| | | preferably W |
| 104 | G | G, A, S, T, D, P, N, E, C, L; |
| | | preferably G |
| 108 | L, M or T; | Q, L⁽⁷⁾, R, P, E, K, S, T, M, |
| | predominantly L | A, H; preferably Q or L⁽⁷⁾ |

Notes:
In particular, but not exclusively, in combination with KERE or KQRE at positions 43-46.
Usually as GLEW at positions 44-47.
Usually as KERE or KQRE at positions 43-46, e.g. as KEREL, KEREF, KQREL, KQREF, KEREG, KQREW or KQREG at positions 43-47. Alternatively, also sequences such as TERE (for example TEREL), TQRE (for example TQREL), KECE (for example KECEL or KECER), KQCE (for example KQCEL), RERE (for example REREG), RQRE (for example RQREL, RQREF or RQREW), QERE (for example QEREG), QQRE, (for example QQREW, QQREL or QQREF), KGRE (for example KGREG), KDRE (for example KDREV) are possible. Some other possible, but less preferred sequences include for example DECKL and NVCEL.
With both GLEW at positions 44-47 and KERE or KQRE at positions 43-46.
Often as KP or EP at positions 83-84 of naturally occurring V_{HH} domains.
In particular, but not exclusively, in combination with GLEW at positions 44-47.
With the proviso that when positions 44-47 are GLEW, position 108 is always Q in (non-humanized) V_{HH} sequences that also contain a W at 103.
The GLEW group also contains GLEW-like sequences at positions 44-47, such as for example GVEW, EPEW, GLER, DQEW, DLEW, GIEW, ELEW, GPEW, EWLP, GPER, GLER and ELEW.

The term “CD28” as used herein refers to the transmembrane co-stimulatory signaling protein expressed on T-cells. CD28 is involved in T-cell activation, proliferation, cytokine production, and survival. An exemplary wild type human CD28 amino acid sequence can be found under NCBI Reference Sequence: NP_006130.1; and Uniprot Reference Number: P10747.

The term “tumor associated antigen” or “TAA” as used herein refers to any antigen that is highly expressed by tumor cells or in the tumor stroma. The term tumor-associated antigen includes TAA that are not completely specific for the tumor but are rather over-expressed on the tumor or its stroma. The term tumor associate antigen also includes tumor specific antigens that are expressed exclusively on the tumor.

As used herein, a “CD28-binding polypeptide” or an “anti-CD28 antibody” refers to any antigen-binding protein having at least one antigen-binding site that specifically binds to CD28. It encompasses antibodies in a divalent form (such as native immunoglobulin molecules or F(ab)′2 fragments) with two CD28-binding sites, as well as antibodies in a monovalent form which have a single CD28-binding site. Herein, a CD28-binding polypeptide typically is an immunoglobulin single variable domain antibody (e.g. VHH)-containing polypeptide having at least one immunoglobulin single variable domain (e.g. VHH domain) that specifically binds to CD28. In certain embodiments, the anti-CD28 antibody or antigen-binding fragment thereof comprises a VHH domain selected from any one of the VHH amino acid sequences of Table 2.

TABLE 2
VHH amino acid sequences.

| VHH ID | VHH sequence | SEQ ID NO: |
|---|---|---|
| 1 | QVQLQESGGGLVQAGGSLRLSCAASGLTFRNYDLGWFRQAPGKERE | 454 |
| | FVAGASWSEEDTYYLNSVKGRFTISRDNAKNTVYLQMNSLRPEDTAI | |
| | YYCAAGLTVNGRLLTRTYEYDHWGQGTQVTVSS | |
| 2 | QVQLQESGGGLVQPGGSLRLSCVASGSISSIDHVGWYRQAPGKERVM | 455 |
| | VAFINSGGRTTYPDAVKGRFTISRDGASNTVFLQMDGLKPDDTAVYY | |
| | CNVLLRDRSGSGRTYWGQGTQVTVSS | |
| 3 | QVQLQESGGGLVQPGGSLRLSCAASERTAITYSMGWFRQAPGKDRDF | 456 |
| | VALISGRDGRTAYADSVKGRFTISQNYAANTVWLQMNSLNPEDTAV | |
| | YYCATSPLVSTDQPDFYSWGQGTQVTVSS | |
| 4 | QVQLQESGGGLVQAGGSLRLSCAASGSIFRSVPVSWYRQAPEKQREF | 457 |
| | VARIFVDGSTHLADPVKGRFTISRDNAKKTVYLQMNSLKPEDTAVYY | |
| | CFMNGDWGQGTQVTVSS | |
| 5 | QVQLQESGGGLVQAGGSLRLSCAATESRFSIKPMGWYRQAPGKQREF | 458 |
| | VATITSPGMANYEDSVKGRFTISKDIPKNTVYLQMNSLKPEDTAVYY | |
| | CRDILSGSWGQGTQVTVSS | |
| 6 | QVQLQESGGGLVQSGGSLRLSCVASGRLLSDNSMTWYRQAPEKQRE | 459 |
| | FVAHITSGGGTNYADSVKGRFTISRDNAKNTVYSQMISLKPEDTAVY | |
| | YCNWPRYGDRGQGTQVTVSS | |
| 7 | QVQLQESGGGLVQAGDSVRLSCAASGRPFSSYAMGWIRQAPGKERE | 460 |
| | FVAAIGGDGSTTRYTESAKGRFTISRDNAKNTMYLQMNSLIPEDTAV | |
| | YYCALDFSLNRIVFGTRADYWGQGTQVTVSS | |
| 8 | QVQLQESGGGLVQGGGSLRLSCAASGRTFSTWHAAWFRQAPGKERE | 461 |
| | FVATIGGSGGSRYYADPVEGRFTISRDNAKNTVYLQMTALKVEDTAV | |
| | YYCATGPAAFGSRKGTSYDYWGQGTQVTVSS | |
| 9 | QVQLQESGGGLVQAGGSLRLSCAASGRAFRINSIGWFRQAPGKEREF | 462 |
| | VAAISWSGRDTYYDDSVKGRFTISRDNAKNTVYLEMNSLKPADTAV | |
| | YSCAARVFFDSGSYAASEYSNWGQGTQVTVSS | |
| 10 | QVQLQESGGGLVQAGGSLRVSCGASGDTFSNYAMAWFRQLAGKER | 463 |
| | EFVAAISWHGGRATYADSVQGRFTISRDNAKNTVYLQMNSLKPEDT | |
| | AVYVCAARLLGGGWSGEEYDYWGQGTQVTVSS | |
| 11 | QVQLQESGGGLVQAGGSLRLSCAFSGRTFSGTAMGWFRQPPGKEREF | 464 |
| | VASIWWSRYATDYADSVKDRFTVSRDNAANTVYLQMNSLKPEDTA | |
| | VYYCAAGHRGYSRFAEAYDYWGQGTQVTVSS | |
| 12 | QVQLQESGGGLVQCGGSLRLSCAASERTFGIRTIGWFRQAPGKEREFV | 465 |
| | GAIKWAGGNTHYADPVKGRFTISRDNAKNTGYLQMNSLKPEDTAVY | |
| | VCAAAKVYYYTPTMGPGSYEFWGQGTQVTVSS | |
| 13 | QVQLQESGGGLVQAGGSLRLSCVVSGRTGSHLDMAWFRQAPGKERE | 466 |
| | FVATISRDGFRIFYADSVKGRFTMSRDNGKNSVYLQMNSLKPEDTAV | |
| | YYCAADAAGFGSRFVSSYDYWGQGTQVTVSS | |
| 14 | QVQLQESGGGLVQAGGSLRLSCAASGRTFDSDRFGMGWFRQAPGKE | 467 |
| | REFVAQINWRGGGAFYADSVKGRFTISRDTVKNTVTLQMNSLQVED | |
| | TAVYFCVADIAAWGARSAASYEYWGRGTQVTVSS | |
| 15 | QVQLQESGGGLVQPGGSLRLSCVASRAISSRWPMGWYRQAPGKQREL | 468 |
| | VAQISHGSITNIMDSVKGRFTISRDYAESTVYLQMNSLKPEDTAVYY | |
| | CYAEDWDTRVQYWGQGTQVTVSS | |
| 16 | QVQLQESGGGLVQAGDSLRLSCAASGLTFSSYTMAWFRQAPGKERE | 469 |
| | FVAAASWSGGSTYYADSVEGRFTISRDYAKNTVYLEMNSLKPEDTA | |
| | VYYCAAEKAPSRTVAAYEYWGQGTQVTVSS | |
| 17 | QVQLQESGGGLVQAGGSLRLSCAASGRTSSGYAMGWFRQAPGKERE | 470 |
| | FVAAIAWSAGSTYYADSVQGRFTISRDKPKITVYLQMDSLTPEDTAV | |
| | YYCAAGTRMPSRMTNAYDYWGRGTQVTVSS | |
| 18 | QVQLQESGGGLVQAGGSLRLSCAASGRTSSGYAMGWFRRAPGKERE | 471 |
| | FVAAISWSGGSTYYADSLRGRFTISRDKPKMTVYLQMDSLTPEDTAV | |
| | YYCAAGTRQVSQTTQAYDYWGRGTQVTVSS | |
| 19 | QVQLQESGGGLAQPGGSLRLSCAASGRPFSSYILGWFRQAPGKEREL | 472 |
| | VAQINWSGDHTYYANSVKGRFTISRDNAENTGYLQMNSLKPEDTAV | |
| | YFCAAKLSAGSSTDTVLHNNRWSWDSWGQGTQVTVSS | |
| 20 | QVQLQESGGGLVQTGGSLRLSCAASGGTYTTWTMGWFRQAPGKDRT | 473 |
| | IVAAIRRTGGDPYYTTSVEGRFTISRDNAKNTVYLQMNSLKPEDTAV | |
| | YYCAASPLWTSSQDDYRHWGQGTQVTVSS | |
| 21 | QVQLQESGGGLVQAGGSLRLSCSASGAYLIVSDMNWYRQSPGKERE | 474 |
| | LVATIGRGGTNYAESVKGRFTISRDNTKNFWYLQMSSLKPEDTAIYY | |
| | CNIVDYWGQGTQVTVSS | |
| 22 | QVQLQESGGGLVQPGGSLRLSCVASGFTFSSYWMYWVRQRPGKGLE | 475 |
| | WVSGMSPEGDMTGYTDSVKGRFTISRDNAKNTLYLQMNSLKSDDTA | |
| | VYSCVKGRTHGSGLYGARDFESRGQGTQVTVSS | |
| 23 | QVQLQESGGGLVQAGGSLRLSCAASGYISSTHFMGWYRQAPGKQRE | 476 |
| | LVAGITGSDLTNYADSVKGRFIISRDNAKNTVALEMNSLKPEDTAVY | |
| | HCRLWGLGLGDGYWGQGTQVTVSS | |
| 24 | QVQLQESGGGVVQSGGSLRLSCAASGFTLSDYWMSWLRQAPGKGLE | 477 |
| | WVSVIKAGDDTTYDLGSVKGRFTISRDNAKNTLYLQMNSLESEDTAV | |
| | YYCARSPYGTYRLDRRYDFRGQGTQVTVSS | |
| 25 | QVQLQESGGGLVQAGGSLTLSCAASGSSSSINAMAWYRQAPGKQRE | 478 |
| | LVAHISRARGDSTIYADSVKGRFTISRENAKNTVYLQMNSLKPEDTAV | |
| | YYCYVAGDRSLDFRSYWGQGTQVTVSS | |
| 26 | QVQLQESGGGLVQAGGSLRLSCAASGTISSTDFMGWYRQAPGKQRE | 479 |
| | LVAGITGSDLTNYADSVKGRFIISRDNAPNTVALQMNSLKPEDTATYY | |
| | CRVWGLGYYYWGQGTQVTVSS | |
| 27 | QVQLQESGGGLVQPGGSLRLSCAASGRTFDYHAVAWFRQAPGKERE | 480 |
| | LVATIGGSGGSRYYADPVLGRFTISRDNAKNTVYLQMNALKPEDTPV | |
| | SYCATGPAGYGSRKSTSYDYWGQGTQVTVSS | |
| 28 | QVQLQESGGGVVQAGGSLRLSCASSEEWFRINNMGWYRQAPGKQRE | 481 |
| | LVAYITPSGSTNYADFVKGRFTISRDNAKKTVLLQMDSLRPEDTAIYY | |
| | CRDISGGSWGQGTQVTVSS | |
| 29 | QVQLQESGGGLVQAGESLRISCAASGRTLTDRTLTDYAVGWFRQPPG | 482 |
| | KEREFVAAIRWSDYRTDYADSVKDRFTvSRDNAKNTVYLQMNSLKP | |
| | EDTAVYYCVAGHRLNSRFAEAYNYWGQGTQVTVSS | |
| 30 | QVQLQESGGGLVQAGGSLRLSCAASERPFSSYAMGWFRQAPGKERE | 483 |
| | FVAAIGGDGSITQYTESAAGRFTISRDNAKNTMYLQMNSLKPEDTAT | |
| | YYCALDFSFNRIVLGSRADYWGQGTQVTVSS | |
| 31 | QVQLQESGGGLVQAGGSLRLSCAASGRTFNAMAWFRQPPGKEREFIA | 484 |
| | AIRWNGYMTDYADSVKGRFTVSRDNAKNTEFLQMNSLKPEDTAVY | |
| | YCAAGDRGSSRFVAAYDYWGQGTQVTVSS | |
| 32 | QVQLQESGGGLVQTGGSLRLSCAASGFTFDDYAIGWFRQAPGKDRE | 485 |
| | GVSCISSSRATTSYADAVKGRFRTSIDNAKKTVYLQTNNLEPEDTAVY | |
| | YCAAGTRMPSRMTNAYDYWGRgTQVTVSS | |
| 33 | QVQLQESGGGLVQPGGSLRLSCVASRAISSRWPMGWYRQAPGKQRE | 486 |
| | LVAQISHGSITNIMDSVKGRFTISRDYAESTVYLQMNSLKPEDTAVYY | |
| | CYAAGQSTAPSASYWGQGTQVTVSS | |
| 34 | QVQLQESGGGLVQPGGSLRLSCAASVRTLTAMGWFRQAPGKERELV | 487 |
| | GSMRWSDGSTYYTDSVKGRFTISRDNAKNTVYLQMNSLKPEDTAVY | |
| | YCAADQVIFYSRKPTDYDYWGQGTQVTVSS | |
| 35 | QVQLQESGGGLVQAGGSLRLSCAASGSISSFNAMGWYRQAPGQQRQ | 488 |
| | LVARITGSGSTNYADSVKGRFTISRVNAKNTVVLQMNSLRSDDTSVY | |
| | LCYADLSTYNAAWNGGVYRNNYWGQGTQVTVSS | |
| 36 | QVQLQESGGGLVQAGGSLRLSCAASGLPFSTYFMAWFRQAPGEEREF | 489 |
| | VASIGGNGGSRYYADPVEGRFTISRDNAKTTVYLQMNALKPEDTAVY | |
| | YCATGPAAFGSRKTSSYDYWGQGTQVTVSS | |
| 37 | QVQLQESGGGLVQAGGSLRLSCAATESRESSKPMGWYRQAPGKQRE | 490 |
| | YVATITSPGMANYADSVRGRFTISKDIPKNTVYLQMDSLKPEDTAVY | |
| | YCRDILSDSWGQGTQVTVSS | |
| 38 | QVQLQESGGGSVQAGGSLRLSCAASGRSFSTYFAAWFRQAPGKEREF | 491 |
| | VATIGGNGGSRYYADPVQGRFTISRDNAKNTVYLQMNALKPEDTAV | |
| | YYCATGPRGFGSRKSTSYDYWGQGTQVTVSS | |
| 39 | QVQLQESGGGLVQPGGSLRLSCAASGFTLSDYWMSWLRQAPGKGLE | 492 |
| | WVSVIASGGSDTYYLNSVKGRFTISRDNDKNTLYLQMNNLKSEDTAV | |
| | YYCARSPYGTYRLDRRYDTRGQGTQVTVSS | |
| 40 | QVQLQESGGGLVQAGGSLRLSCAASGYISSTHFMGWYRQAPGKQRE | 493 |
| | LVAGITGSDLTNYADSVKGRFIISRDNAKNTVALEMNSLKPEDTAVY | |
| | YCRLWGLGLGDGYWGQGTQVTVSS | |
| 41 | QVQLQESGGGLVQAGGSLGLSCAASGSIPSTHFMGWYRQPPGKQREL | 494 |
| | VAGITGSDLTNYADSVKGRFIISREHAQNTVALQMNSLKPEDTAVYY | |
| | CRVWGVGYDYWGQGTQVTVSS | |
| 42 | QVQLQESGGGLVQAGDSVRLSCAASGRPFSSYAMGWIRQAPGKERE | 495 |
| | FVAAIGGDGSTTRYTESAKGRFTISRDNAKNTMYLRMNSLIPEDTAV | |
| | YYCALDFSLNRIVFGTRADYWGQGTQVTVSS | |
| 43 | QVQLQESGGGLVQSGGSLRLSCVVSGRLISADSITWYRQAPEKQREF | 496 |
| | VAHITSGGGTNYADFVKGRFAISRDNAKNTVYLQMNSLKPDDTAVY | |
| | YCHWPRYGDWGQGTQVTVSS | |
| 44 | QVQLQESGGGLVQAGGSLRLSCAASGLTFSSYSMGWFRQAPGKEREF | 497 |
| | VGAISWSNGRTYYADFVKGRFTISRDNAKNTVYMQMNSLKPEDTAV | |
| | YYCAAEKAPSRTVAAYEYWGQGTQVTVSS | |
| 45 | QVQLQESGGGLLQAGESLRLSCAASGSSSSINAMAWSRQAPGKQRE | 498 |
| | MVAHISRARGDSTIYADSVKGRFTISRENAKNTVYLQMNNLKPEDTA | |
| | VYFCYVAGNRDLDFRSYWGQGTQVTVSS | |
| 46 | QVQLQESGGGLVQAGGSLRLSCAASGSISSTDFMGWYRQAPGKQRE | 499 |
| | LVAGITGSDLTNYADSVKGRFIISREHAGNTMALQMNSLKPEDTAVY | |
| | YCRVWGLGYAYWGQGTQVTVSS | |
| 47 | QVQLQESGGGLVQAGGSLRLSCAASGLTFSSYSMGWFRQAPGKEREF | 500 |
| | VGAISWSGGRTYYADSVTGRFTISRDNAKNTVYLQMNNLKPEDTGV | |
| | YYCAAERAPSRQVAAYEFWGQGTQVTVSS | |
| 48 | QVQLQESGGGLVQAGGSLRLSCGASGRTVSNYAMGWFRQAAGKER | 501 |
| | EFVAAVAWTGGRTTYADSVKGRFTLSRNSAKDTVYLQMNSLKPEDT | |
| | AVYYCAARLLGGGWSGEEYDSWGQGTQVTVSS | |
| 49 | QVQLQESGGGLVQPGGSLRLSCVTSRSISSRWPMGWYRQAPGKQREL | 502 |
| | VAQISHGSIINIMDSVKGRFTISRDYAESTVYLQMNSLKPEDTAVYTC | |
| | YAEDWDTRVQYWGQGTQVTVSS | |
| 50 | QVQLQDSGGGLVQPGGSLGLSCAASGSIPSTHFMGWYRQPPGKQREL | 503 |
| | VAGITGSDLTNYADSVKGRFIISREHAQNTVALQMNSLKPEDTAVYY | |
| | CRVWGVGYDYWGQGTQVTVSS | |
| 51 | QVQLQESGGGLVQAGGSLRLSCLASERTFSTYFKAWFRQAPGKEREF | 504 |
| | VATIGGNGGSRYYAEPVEGRFFISRDNAKNTVYLEMNALKPEDTAVY | |
| | YCATGPRGFGSRKSTSYDYWGQGTQVTVSS | |
| 52 | QVQLQESGGGLVQAGGSLRLSCAASGRIFSTYSMGWFRQAPGKEREF | 505 |
| | VATIGGSGGSRYYADPVEGRFTISRDNAKNTMYLQMNALKPEDTAV | |
| | YYCATGPRGFGSRKTTSYDYWGQGTQVTVSS | |
| 53 | QVQLQESGGGLVQAGNSLRLSCVASGRTVSSYAMGWFRQALGKERE | 506 |
| | FVAAMSWTGGRTTYADSVKGRFTMSRDNAKSTAYLQMNNLKPEDT | |
| | AVYYCAGRLLGGGWSGEEYGYWGQGTQVTVSS | |
| 54 | QVQLQESGGGLVQDGGSLRLSCAASGRAFSTYFMAWFRQGPGKERE | 507 |
| | FVATIGGNGGSRYYADPVEGRFTISRDNAKNTMYLQMNALKPEDTD | |
| | VYYCETGPRGFGSRKNISYDYWGQGTQVTVSS | |
| 55 | QVQLQESGGGLLQAEGSLRLSCAASGLTFSSYSMGWFRQAPGKEREF | 508 |
| | VGAISWSGGRTYYADSVQGRFTISRDNSKNTVYLQMNSLKPEDTAVY | |
| | YCAAERAPSRQVAAYEYWGQGTQVTVSS | |
| 56 | QVQLQESGGGLVQAGGSLRLSCAASGFTFSTYSMGWFRQAAGKERE | 509 |
| | FVGAISWSNGRTYYADSVEGRFTISRDNAKNTVYLQMNSLKPEDTAV | |
| | YYCAAEKAPSRKVSAYEYWGQGTQVTVSS | |
| 57 | QVQLQESGGGLVQAGGSVRLSCLASERTFSTYFKAWFRQAPGKEREF | 510 |
| | VATIGGNGGSRYYAEPVEGRFFISRDNAKNTVYLEMNALKPEDTAVY | |
| | YCATGPRGFGSRKSTSYDYWGQGTQVTVSS | |
| 58 | QVQLQESGGGLVPAGGSLRLSCAASGLTFSSYSMGWFRQAPGKEREF | 511 |
| | VGAISWSGGRTYYADSVTGRFTISRDNAKNTVYLQMNNLKPEDTGV | |
| | YYCAAERAPSRQVAAYEFWGQGTQVTVSS | |
| 59 | QVQLQDSGGGLVQAGGSLGISCAAYGSIPSTHFMGWYRQPPGKQREL | 512 |
| | VAGITGSDLTNYADSVKGRFIISREHAQNTVALQMNSLKPEDTAVYY | |
| | CRVWGVGYDYWGQGTQVTVSS | |
| 60 | QVQLQESGGGLVQAGGSLRLSCAASGRTTSTYFMAWFRQAPGKERE | 513 |
| | FVATIGGSGGSRHYADPVQGRFTISRDNAKSTMYLQMNALKPEDTAA | |
| | YYCATGPRGFGSRKSTSYDYWGQGTQVTVSS | |
| 61 | QVQLQESGGGLVQAGGSLRLSCAVSGSIPSTHFMGWYRQPPGKQREL | 514 |
| | VAGITGSDLTNYADSVKGRFIISREHAKNTVALQMNSLKPEDTAVYY | |
| | CRVWGVGYDYWGQGTQVTVSS | |
| 62 | QVQLQESGGGLVQAGDSLRVSCGASGGTFSSYAMAWFRQAAGKERE | 515 |
| | FVAAIAWHGGRTSYADSVRGRFTISRDNAKNTGYLQMNSLKPADTA | |
| | VYYCAARLLGGGWSGEEYEYWGQGTQVTVSS | |
| 63 | QVQLQESGGGVVQAGGSLTLSCAASGSSSSINAMAWYRQAPGKQRE | 516 |
| | LVAHISRARGDSTIYADSVKGRFTISRENAKNTVYLQMNSLKPEDTAV | |
| | YYCYVAGDRSLDFRSYWGQGTQVTVSS | |
| 64 | QVQLQESGGGLVQAGGSLRLSCAASGRSFSTYFMAWFRQAPGKERE | 517 |
| | FVATIGGNGGSRYYADPVSGRFTISRDNAKNTVYLQMNALKPEDTAV | |
| | YYCATGPRGFGSRKSTSYDYWGQGTQVTVSS | |
| 65 | QVQLQESGGGLVQAGGSLRLSCAASGRTVSTYFMTWFRQAPGKERE | 518 |
| | FVATIGGSGGSRYYADPVQGRFTISRDNAKNTVYLQMNALKPEDTAV | |
| | YYCATGPAGFGSRKSTSYDYWGQGTQVTVSS | |
| 66 | QVQLQESGGGLVQAGDSLRLSCAASGLTFSSYSMGWFRQAPGKEREF | 519 |
| | VGAISWSGGRTYYADSVQGRFTISRDNSKNTVYLQMNSLKPEDTAVY | |
| | YCAAERAPSRQVAAYEYWGQGTQVTVSS | |
| 67 | QVQLQESGGGSVQAGGSLLSCAACGSIASTHFMGWYRQAPGKQREL | 520 |
| | VAGLTESSLTNYADSVKGRFIISREHAKNTVALQMNSLKPEDTAVYY | |
| | CGVWGLGFAYWGQGTQVTVSS | |
| 68 | QVQLQESGGGLVQAGGSLRLSCAYSERTFDTYTMAWFRQAPGKERE | 521 |
| | FVATIGGSGGSRYYTDPVMGRFTISRDNAKNTVYLQMNALKPEDTAV | |
| | YYCATGPAGFGSRKTTSYDYWGQGTQVTVSS | |
| 69 | QVQLQESGGGLVQAGGSLRLSCAASGLTFSSYSMGWFRQAPGKEREF | 522 |
| | VGAISWSAGRTYYADSVQGRFTISRDNSKNTVYLKMNSLKPEDTAKY | |
| | YCAAERAPSRQVAAYEYWGQGTQVTVSS | |
| 70 | QVQLQESGGGLVQAGASLRLSCAASGLTFSSYSMGWFRQAPGKEREF | 523 |
| | VGAISWSAGRTYYADSVQGRFTISRDNSKNTVYLQMNSLKPEDTAKY | |
| | YCAAERAPSRQVAAYEYWGQGTQVTVSS | |
| 71 | QVQLQESGGGFVQPGGSLRLSCVTSRSISSRWPMGWYRQAPGKQREL | 524 |
| | VAQISHGSIINIMDSVKGRFTISRDYAESTVYLQMNSLKPEDTAVYTC | |
| | YAEDWDTRVQYWGQGTQVTVSS | |
| 72 | QVQLQESGGGLVQAGDSVRLSCAASGRPFSSYAMGRIRQAPGKEREF | 525 |
| | VAAIGGDGSTTRYTESAKGRFTISRANAKNTMYLQMNSLIPEDTAVY | |
| | SCALDFSLNRIVFGTRADYWGQGTQVTVSS | |
| 73 | QVQLQESGGGLVQAGGSLRLSCAASGLTFSSYSLGWFRQAPGKEREF | 526 |
| | VGAISWSGGRTYYADSVTGRFTISRDNAKNTVYLQMNNLKPEDTGV | |
| | YYCAAERAPSRQVAAYEFWGQGTQVTVSS | |
| 74 | QVQLQESGGGLVQAGGSLRLSCAATGDRFSIKPMGWYRQAPGKQRE | 527 |
| | FVATITSPGTANYEDSVKGRFTISKDIAKNTVYLQMNSLKPEDTGVYY | |
| | CRDILSDSWGQGTQVTVSS | |
| 75 | QVQLQESGGGLVQAGGSLRLSCAASGRTFSNYDMGWFRQAPGKERE | 528 |
| | FVAACSWGGENTAYYVNSVKGRFTISRDNAKNTVYLQMNSLKPEDT | |
| | AIYYCVAGLTVNGRLLTRTYEYDNWGQGTQVTVSS | |
| 76 | QVQLQESGGGLVQAGGSLRLSCAASGRTFSSTAMGWFRQPPGKEREF | 529 |
| | VAAIRWSDYRTDYADSVKDRFTVSRDNAKNTVYLQMNSLKPEDTAV | |
| | YYCVAGHRLNSRFAEAYDYWGQGTQVTVSS | |
| 77 | QVQLQESGGGLVQAGGSLRLSCAASGRAFSTYHVAWFRQAPGKERE | 530 |
| | FVATIGGSGGSRYYADPVKGRFTISRDNAKNTVYLQMNALKPEDTAV | |
| | YYCATGPAGFGSRRSTSYDYWGQGTQVTVSS | |
| 78 | QVQLQESGGGLVQAGGSLRLSCAASGSISSTHFMGWYRQAPGKQRE | 531 |
| | LVAGITESSLTSYATSVKGRFIISREHAKNTVALQMNSLEPEDTAVYY | |
| | CGVWGLGYAYWGQGTQVTVSS | |
| 79 | QVQLQESGGGLVQAGGSLRLSCAASGRTTSTYFMAWFRQAPGKERE | 532 |
| | FVATIGGSGGSRHYADPVQGRFTISRDNAKSTMYLQMNALKPEDTAV | |
| | YYCATGPRGFGSRKSTSYDYWGQGTQVTVSS | |
| 80 | QVQLQESGGGLVQAGGSLRLSCAATGSRFSTKPMGWYRQAPGKQRD | 533 |
| | YVATITTPGMANYAASVKGRFTISKDITKNTVYLQMNSLKPEDTATY | |
| | YCRDILSDDWGQGTQVTVSS | |
| 81 | QVQLQESGGGLVQPGGSLRLSCAASGRSFSTYFMAWFRQAPGKEREF | 534 |
| | VATIGGNGGSRYYADPVEGRFTISRDNAKNTVYLQMNALKPEDTAV | |
| | YYCATGPRGFGSRKSTSYDYWGQGTQVTVSS | |
| 82 | QVQLQESGGGLVQAGGSLRLSCAASGRTFSSTAMGWFRQPPGEEREF | 535 |
| | VAAIRWSDYRTDYADSVKDRFTVSRDNAKNTVYLQMNSLKPEDTAV | |
| | YYCVAGHRLNSRFAEAYDYWGQGTQVTVSS | |
| 83 | QVQLQESGGGLVQAGGSLRLSCAASGLPFSTYFMAWFRQAPGKEREF | 536 |
| | VASIGGNGGSRYYADPVEGRFTISRDNAKTTVYLQMNALKPEDTAVY | |
| | YCATGPAAFGSRKTSSYDYWGQGTQVTVSS | |
| 84 | QVQLQESGGGLVQAGESLRLSCAASGRSFSTDFMAWFRQAPGKEREF | 537 |
| | VATIGGNGGSRYYADPVQGRFTISRDNAKNTVYLQMNALKPEDTAV | |
| | YYCATGPRGFGSRKSTSYDYWGQGTQVTVSS | |
| 85 | QVQLQESGGGLVQSGGSLRLSCVASGRLISANSMGWYRQAPEKQREF | 538 |
| | VAHITSGGGTSYADSVKGRFTISRDNAKNTVYLQMNSLKPEDTAVYY | |
| | CNWPRYGDWGQGTQVTVSS | |
| 86 | QVQLQESGGGLVQAGGSLRLSCAASGLTFSSYSMGWFRQAPGKEREF | 539 |
| | VGAISWSAGRTYYSDSVEGRFTISRDNAKNTVYLQMNSLKAEDTAV | |
| | YYCAAERAPSRRVDAYEYWGQGTQVTVSS | |
| 87 | QVQLQESGGGLVEAGGSLRLSCAASGRSFSTYFAAWFRQAPGKEREF | 540 |
| | VATIGGNGGSRYYADPVQGRFTISRDNAKNTVYLQMNALKPEDTAV | |
| | YYCATGPRGFGSRKSTSYDYWGQGTQVTVSS | |
| 88 | QVQLQESGGGLVQPGGSLRLSCAASGLTFSSYSMGWFRQAPGKEREF | 541 |
| | VGAISWSGGRTYYADSVTGRFTISRDNAKNTVYLQMNNLKPEDTGV | |
| | YYCAAERAPSRQVAAYEFWGQGTQVTVSS | |
| 89 | QVQLQESGGGLVQAGGSLRLSCAASGLTFSSYSMGWFRQAPGKEREF | 542 |
| | VGAISWSYGSTYYADSVKGRFAISRDNAQNTVYLQMNSLKPEDTAV | |
| | YYCAAEKAPSRRVAAYEYWGQGTQVTVSS | |
| 90 | QVQLQESGGGLVQAGGSLRLSCAASGRSFSTYFMAWFRQAPGKERE | 543 |
| | FVATIGGNGGSRYYADPVEGRFTISRDNAKNTVYLQMNALKPEDTAV | |
| | YYCATGPRGFGSRKSTSYDYRGQGTQVTVSS | |
| 91 | QVQLQESGGGLVQAGGSLRLSCAASGYISSTHFMGWYRQAPGEQRE | 544 |
| | LVAGITGSDLTNYADSVKGRFIISRDNAKNTVALEMNSLKPEDTAVY | |
| | YCRLWGLGLGDGYWGQGTQVTVSS | |
| 92 | QVQLQESGGGLVQAGGSLRLSCAASGRTFSSTAMGWFRQPPGKEREF | 545 |
| | VAAIRWSDYRTDYADSVKDRFTVSRDNAKNTVYLQMNSLKPEDTAV | |
| | YYCVAGHGLNSRFAEAYDYWGQGTQVTVSS | |
| 93 | QVQLQESGGGLVQAGGSLRLSCAARGLTFSSYSMAWFRQAPGKERE | 546 |
| | FVGAISWSAGRTYYSDSVEGRFTITRDNAKNTVYLQMNSLKPEDTAV | |
| | YYCAAERAPSRRVDAYEYWGQGTQVTVSS | |
| 94 | QVQLQESGGGLVQAGGSLRLSCAASGLTFSSYSMGWFRQAPGKEREF | 547 |
| | VGAISWSAGRTYYADSVQGRFTISRDNSKNTVYLQMNSLKPEDTAKY | |
| | YCAAERAPSRQVAAYEYWGQGTQVTVSS | |
| 95 | QVQLQESGGGLVQAGGSLRLSCAASGGTFSTYFMTWFRQAPGKERE | 548 |
| | FVATIGGSGGSRHYADPVQGRFTISRDNAKSTMYLQMNALKPEDTAV | |
| | YYCATGPRGFGSRKSTSYDYWGQGTQVTVSS | |
| 96 | QVQLQESGGGLAQPGGSMRLSCAASGRPFSSYILGWFRQAPGKEREL | 549 |
| | VSQINWSGDHTYYANSVKGRFTISRDNAENTGYLQVNSLKPEDTAVY | |
| | FCAAKLSAGSSTDTVLHNNRWSWDYWGQGTQVTVSS | |
| 97 | QVQLQESGGGLVQPGGSLRLSCAASGLPFSTYFMAWFRQAPGKEREF | 550 |
| | VASIGGNGGSRYYADPVEGRFTISRDNAKTTVCLQMNALKPEDTAVY | |
| | YCATGPAAFGSRKTSSYDYWGQGTQVTVSS | |
| 98 | QVQLQESGGGLVQAGGSLRLSCAASERTAFTYSMGWFRQAPGKDRD | 551 |
| | FVALISGRDGRTAYADSVKGRFTISQNYAANTVWLQMNSLKPEDTA | |
| | VYYCATSPLVSTDQPDFYSWGQGTQVTVSS | |
| 99 | QVQLQESGGGLVQAGDSLRLSCAASGLTFSSYTMAWFRQAPGKERE | 552 |
| | FVAAASWSGGNTYYADSVEGRFTISRDYAKNTVYLEMNSLKPEDTA | |
| | VYYCAAEKAPSRTVAAYEYWGQGTQVTVSS | |
| 100 | QVQLQESGGGLVQAGGSLRLSCSASGRSFITYFMAWFRQFPGKEREF | 553 |
| | VATIGGNGGSRYYADPVQGRFTISRDNAKNTGYLQMNALQPEDTAV | |
| | YYCATGPRGFGSRKSTSYDYRGQGTQVTVSS | |
| 101 | QVQLQESGGGLVQAGGSLRLSCAASGRSFITYFMAWFRQAPGREREF | 554 |
| | VATIGGNGGSRYYADPVEGRFTISRDNAKNTLYLQMNALEPEDTAIY | |
| | YCATGPRGFGSRKSNSYDYWGQGTQVTVSS | |
| 102 | QVQLQESGGGLVQAGGSLRLSCAASGRSFITYFMAWFRQTPGKEREF | 555 |
| | VATIGGNGGSRYYADPVEGRLTISRDNAKNTVYLQMNALKPEDTAV | |
| | YYCATGPRGFGSRKSTSYDYWGQGTQVTVSS | |
| 103 | QVQLQESGGGLVQAGGSLTLSCAASGRSFITYFMAWFRQAPGKEREF | 556 |
| | VATIGGNGGSRYYADPVQGRFTISRDNAKNTMYLQMNALKPEDTAV | |
| | YYCATGPRGFGSRKSTSYDYWGQGTQVTVSS | |
| 104 | QVQLQESGGGLVQAGGSLSLSCAASGSITSFNAMGWYRQAPGQQRQ | 557 |
| | LVARITGSGSTNYADSVKGRFTISRVGAKNTVVLQMNSLKSEDTSVY | |
| | LCYADLSTYNAEWNGGAYRNNYWGQGTQVTVSS | |
| 105 | QVQLQESGGGLVQAGGSLRLSCAASGSSSSINAMAWYRQAPGKQRE | 558 |
| | MVAHISRARGDSTIYADSVKGRFTISRENAKNTVYLQMNNLKPEDTA | |
| | AYFCYVAGNRDLDFRSYWGQGTQVTVSS | |
| 106 | QVQLQESGGGLVQAGGSLRLSCAASGLTFSSYSMGWFRQAPGKEREF | 559 |
| | VGAISWSGGRTYYADSVQGRFTISRDNSKNTVYLQMNSLKPEDTAVY | |
| | YCAAERAPSRQVAAYEYWGQGTQVTVSS | |
| 107 | QVQLQESGGGLVQAGDSLRLSCAASGPTFGTYAMGWFRQAPGKERD | 560 |
| | FVAAMSWSNGRTYYADSVEGRFTISRDNAKNTVYLQMNSLKPEDTA | |
| | VYYCAAEKAPSRKVSAYEYWGQGTQVTVSS | |
| 108 | QVQLQESGGGLVQAGGSLRLSCAASGRTFSSTAMGWFRQPPGKEREF | 561 |
| | VAAIRWSDYRTDYADSVKDRFTVSRDNAKNTVYLQMNSLEPEDTAV | |
| | YYCVAGHRLNSRFAEAYDYWGQGTQVTVSS | |
| 109 | QVQLQESGGGLVQAGGSLRLSCAASGRSFISYFMAWFRQAPGTEREF | 562 |
| | VATIGGNGGSRYYADPVEGRFTISRDNAKNTLYLQMNALKPEDTAV | |
| | YYCATGPRGFSSRKSTSYDYWSQGTQVTVSS | |
| 110 | QVQLQESGGGLVQGGGSLRLSCAASGRTFSTWHAAWFRQAPGKERE | 563 |
| | FVATIGGSGGSRYYADPVEGRFTISRDNAKNTVYLQMTALKVEDTAV | |
| | YYCATGPAAFGSRKSTSYDYWGQGTQVTVSS | |
| 111 | QVQLQESGGGLVQAGGSLRLSCAYSERTFDTYTMAWFRQAPGKERE | 564 |
| | FVATIGGSGGSRYYTDPVMSRFTISRDNAKNTVYLQMNALKPEDTAV | |
| | YYCATGPAGFGSRKTTSYDYWGQGTQVTVSS | |
| 112 | QVQLQESGGGLVQAGGSLRLSCAASGRTSSGYAMGWFRQAPGKERE | 565 |
| | FVAAIAWSAGSTYYADSVQGRFTISRDKPKIMVYLQMDSLTPEDTAV | |
| | YYCAAGTRMPSRMTNAYDYWGRGTQVTVSS | |
| 113 | QVQLQESGGGLVQAGGSLRLSCAASGRTFSSTAMGWFRRPPGKEREF | 566 |
| | VAAIRWSDYRTDYADSVKDRFTVSRDNAKNTVYLQMNSLKPEDTAV | |
| | YYCVAGHRLNSRFAEAYDYWGQGTQVTVSS | |
| 114 | QVQLQESGGGLMQAGGSLRLSCAASGRTVSTYFMSWFRQAPGKERE | 567 |
| | FVATIGGSGGSRYYADPVQGRFTISRDNAKNTVYLQMTALKPEDTAV | |
| | YYCATGPAGFGSRKTTSYDYWGQGTQVTVSS | |
| 115 | QVQLQESGGGLVQAGGSLRLSCAAFGRTFSSTAMGWFRQPPGKEREF | 568 |
| | VAAIRWSDYRTDYADSVKDRFTVSRDNAKNTVYLQMNSLKPEDTAV | |
| | YYCVAGHRLNSRFAEAYDYWGQGTQVTVSS | |
| 116 | QVQLQESGGGLVQAGGSLRLSCAASGRTFSSTAMGWFRQAPGKERE | 569 |
| | FVAAIRWSDYRTYYADSVKDRFTVSRDNAKNTVYLQMNSLKPEDTA | |
| | VYYCVAGHRLITRITEAYDYWGQGTQVTVSS | |
| 117 | QVQLQESGGGLVQSGGSLRLSCVVSGRLISADSMTWYRQAPEKQREF | 570 |
| | VAHITSGGGTNYADSVQGRFTISRDNAKNTVYLQMNSLKPEDTAVY | |
| | YCNCPRYGDWGQGTQVTVSS | |
| 118 | QVQLQESGGGLVQAGDSLRLSCAASGLTFSSYTMAWFRQAPGKERE | 571 |
| | FVAAASWSGGSTYYADSVEGRFTISRDYAKNTVYLEMNSLKPEDTA | |
| | VYYCAAEKAPSQTVAAYEYWGQGTQVTVSS | |
| 119 | QVQLQESGGGLVQSGGSLRLSCVASGRLISANSMGWYRQAPEKQREF | 572 |
| | VAHITSGGGTNYADSVKGRFTISRDNAKNTVYLQMNSLKPEDTAVY | |
| | YCNWPRYGDWGQGTQVTVSS | |
| 120 | QVQLQESGGGLVQAGGSLRLSCAASGRAFRINSIGWFRQAPGKEREF | 573 |
| | VAAISWSGRDTYYDDSVKGRFTISRDNAKNTVYLEMNSLKPADTAV | |
| | YSCAARVFFDSGSYAASEYSNWGQGTQVTVFSS | |
| 121 | QVQLQESGGGLVQAGGSLRLSCAASGRTTSTYFMAWFRQAPGKERE | 574 |
| | FVATIGGSGGSRHYADPVQGRFTISRDNAKSTMYLQMNALKPEDTAV | |
| | YYCATGPRGFGSRRSTSYDYWGQGTQVTVSS | |
| 122 | QVQLQESGGGLVQAGGSLRLSCAASGSSSSINAMAWYRQAPGKQRE | 575 |
| | MVAHISRARGDSTIYADSVKGRFTISRENAKNTVYLQMNNLKPEDTA | |
| | VYFCYVAGNRDLDFRSYWGQGTQVTVSS | |
| 123 | QVQLQESGGGLVQAGGSLRLSCSASERTFSTYFEAWFRQTPGKEREF | 576 |
| | VATIGGNGGSRYYAEPVEGRFFISRDNAKNTVYLEMSALKPEDTAVY | |
| | YCATGPRGFGSRKSTSYDYWGQGTQVTVSS | |
| 124 | QVQLQESGGGLVQAGDSLRLSCAASGRTSSTYFMTWFRQAPGKEREF | 577 |
| | VATIGGSGGSRHYADPVQGRFTISRDNAKSTMYLQMNALKPEDTAV | |
| | YYCATGPRGFGSRKSTSYDYWGQGTQVTVSS | |
| 125 | QVQLQESGGGLVQAGGSLRLSCAASGRPFSSYAMGWIRQAPGKEREF | 578 |
| | VAAIGGDGSTTRYTESAKGRFTISRDNAKNTMYLQMNSLIPEDTAVY | |
| | YCALDFSLNRIVFGTRADYWGQGTQVTVSS | |
| 126 | QVQLQESGGGLVQAGGSLRLSCAASATISSTDFMGWYRQAPGKQRE | 579 |
| | LVAGITSSDLTNYADSVKGRFIISRDNAKNTVALQMNSLKPEDTATYY | |
| | CRVWGLGYYYWGQGTQVTVSS | |
| 127 | QVQLQESGGGLVQAGGSLRLSCAASGRTFSMYNMGWFRQAPGKERE | 580 |
| | FVATIGGNGGSRYYADPVEGRFTISRDNAKNTLYLQMNALEPEDTAI | |
| | YYCATGPRGFGSQKSNSYDYWGQGTQVTVSS | |
| 128 | QVQLQESGGGLVQAGGSLRLSCAASGRTVSTYFMSWFRQAPGKERE | 581 |
| | FVATIGGSGGSRHYADPVQGRFTISRDNAKNTVYLQMTALKPEDTAV | |
| | YYCATGPAGFGSRKTTSYDYWGQGTQVTVSS | |
| 129 | QVQLQESGGGLVRAGGSLRLSCAASFSSSYMGWFRQAPGKEREFVA | 582 |
| | TIGGNGGSRYYADPVEGRFTISRDNAKNTVYLQMNALRPEDTAVYY | |
| | CATGPRGFGSRKSTSYDYWGQGTQVTVSS | |
| 130 | QVQLQESGGGLVQAGGSLRLSCAASGRAFSTYFMAWFRQGPGKERE | 583 |
| | FVATIGGSGGSRYYADPVEGRFTISRDNAKNTMYLQMNALKPEDTAV | |
| | YYCATGPRGFGSRKTTSYDYWGQGTQVTVSS | |
| 131 | QVQLQESGGGLVQAGGSLRLSCGASGRTFSSYAVGWFRQAAGKERE | 584 |
| | FVGAISWTGGRTTYADSVKGRFTISRDNAKNTLYLQMNSLKPEDTAV | |
| | YYCTARLLGGGWSGEEYDYWGQGTQVTVSS | |
| 132 | QVQLQESGGGLVQAGGSLRLSCAASGRSFITYFMAWFRQAPGKEREF | 585 |
| | VATIGGNGGSRYYADPVEGRFTISRDNAKNTVYLQMNALKPEDTAV | |
| | YYCATGPRGFGSRKSTSYDYWGQGTQVTVSS | |
| 133 | QVQLQESGGGLVQAGGSLRLSCGASGRTASSYAMGWFRQAAGKERE | 586 |
| | FVAAMPWTGGRTTYADSVKGRFTISRDNAKNTVFLQMNSLKPEDTG | |
| | VYYCAARLLGGGWSGEEYDYWGQGTQVTVSS | |
| 134 | QVQLQESGGGLVQTGGSLRLSCAASGRSFSTYFMAWFRQFPGKEREF | 587 |
| | VATIGGNGGSRYYADPVQGRFTISRDNAKNTGYLQMNALQPEDTAV | |
| | YYCATGPRGFGSRKSTSYDYWGQGTQVTVSS | |
| 135 | QVQLQESGGGLVRAGGSLRLSCAASGLTFSSYSMGWFRQAPGKEREF | 588 |
| | VGAISWSGGRTYYADSVTGRFTISRDNAKNTVYLQMNNLKPEDTGV | |
| | YYCAAERAPSRQVAAYEFWGQGTQVTVSS | |
| 136 | QVQLQESGGGLVQAGGSLRLSCAASGSISSTHFMGWYRQAPGKQRE | 589 |
| | LVAGITESSLTNYADSVKGRFIISREHAKNTVALQMNSLEPEDTAVYY | |
| | CGVWGLGYAYWGQGTQVTVSS | |
| 137 | QVQLQESGGGLVQAGGSLRLSCAASGSIASTHFMGWYRQAPGKQRE | 590 |
| | LVAGLTESSLTNYADSVKGRFIISREHAKNTVALQMNSLKPEDTAVY | |
| | YCGVWGLGFAYWGQGTQVTVSS | |
| 138 | QVQLQESGGGLVQAGGSLRLSCAASGSIFSTNIMGWYRQAPGKQREL | 591 |
| | VAGITGSDLTNYADSVKGRFIISRDNAPNTVALRMNSLKPEDTATYYC | |
| | RVWGLGYYYWGRGTQVTVSS | |
| 139 | QVQLQESGGGLVQAGGSLRLSCAASGGTFSTYFMTWFRQAPGKERE | 592 |
| | FVATIGGSGGSRHYADPVQGRFTISRDNAKSTMYLQMNALEPEDTAV | |
| | YYCATGPRGFGSRKSTSYDYWGQGTQVTVSS | |
| 140 | QVQLQESGGGLVQAGGSLRLSCTASGSISSTHFMGWYRQAPGNQREL | 593 |
| | VAGITTSSATSYADSVKGRFIISREHAKNTVALQMNSLKPEDTAVYYC | |
| | GVWGVGYAYWGQGTQVTVSS | |
| 141 | QVQLQESGGGLVQAGGSLRLSCAASGYISSTHFMGWYRQAPGKQRE | 594 |
| | LVAGITGSDLTNYADSVKGRFIISRDNAKNTVALEMNSLKPEDTAVY | |
| | YCRLWGLGLGYGYWGQGTQVTVSS | |
| 142 | QVQLQESGGGLVQAGGSLRLSCAASGRPFSSYAMGWFRQAPGKERE | 595 |
| | FVAAIGGDGSITQYTESAKGRFTISRDNAKNTIYLQMNSLKPEDTAVY | |
| | VCALDESFNRIVLGTRADYWGQGTQVTVSS | |
| 143 | QVQLQESGGGLVQAGGSLRLSCSASERTESTYFKAWFRQAPGKGREF | 596 |
| | VATIGGNGGSRYYADPVKGRFFISRDNAKNTVYLEMNALKPEDTAV | |
| | YYCATGPRGFGSRKSTSYDYWGQGTQVTVSS | |
| 144 | QVQLQESGGGLVQAGGSLRLSCAASGRTFSNYDLGWFRQAPGKERE | 597 |
| | FIAACGWKAEDTYYLNSVKGRFTISRDNAKNTVTLQMNSLNPEDTAI | |
| | YYCAAGLTVNGRLLTRTYEYDIWGQGTQVTVSS | |
| 145 | QVQLQESGGGLVQAGGSLRLSCAASGRTVSTYFMSWFRQAPGKERE | 598 |
| | FVATIGGSGGSRYYADPVKGRFTISRDNAKNTVYLQMTALEPEDTAV | |
| | YYCATGPAGFGSRKSTSYDYWGQGTQVTVSS | |
| 146 | QVQLQESGGGLVQAGGSLRLSCAASGRSFSTYFMAWFRQAPGKERE | 599 |
| | FVATIGGNGGSRYYADPVEGRFTISRDNAKNTLYLQMNALEPEDTAI | |
| | YYCATGPRGFGSRKSNSYDYWGQGTQVTVSS | |
| 147 | QVQLQESGGGLVQAGGSLRLSCAASGRSFSTYFMAWFRQAPGKERE | 600 |
| | FVATIGGNGGSRYYADPVEGRFTISRDNAKNTVYLQMNALKPEDTAV | |
| | YYCATGPRGFGSRKSTSYDYWGQGTQVTVSS | |
| 148 | QVQLQESGGGLVQAGGSLRLSCAASGGTYTTYTMGWFRQAPGKDRT | 601 |
| | IVAAIRRTGGDPYYSTSVKGRFTISRDNAKNTVYLQMNSLKPEDTAV | |
| | YYCAGSPLWTSSQDDYRHWGQGTQVTVSS | |
| 149 | QVQLQESGGGLVQAGGSLRLSCSASERTFSTYFKVWFRQAPGKEREF | 602 |
| | VATIGGNGGSRYYADSVEGRFFISRDNAKNTVYLEMNALKPEDTAVY | |
| | YCATGPRGFGSRKSTSYDYWGQGTQVTVSS | |
| 150 | QVQLQESGGGLVQAGGSLGLSCAAYGSIPSTHFMGWYRQPPGKQRE | 603 |
| | LVAGITGSDVTNYADSVKGRFIISREHAQNTVALQMNSLKPEDTAVY | |
| | YCRVWGVGYDYWGQGTQVTVSS | |
| 151 | QVQPQESGGGLVQAGGSLGLSCAASGSIPSTHFMGWYRQPPGKQREL | 604 |
| | VAGITGSDLTNYADSVKGRFIISREHAQNTVALQMNSLKPEDTAVYY | |
| | CRVWGVGYDYWGQGTQVTVSS | |

The CD28-binding polypeptides provided herein include monovalent and multivalent (e.g., bivalent) constructs. In some embodiments, a CD28-binding polypeptide provided herein contains one or two immunoglobulin single variable domains (e.g., VHH domains) that each individually binds CD28.

In certain embodiments, the anti-CD28 antibody or antigen-binding fragment thereof comprises a VHH domain that is at least about 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or more identical to the amino acid sequence of any one of the VHH sequences recited in Table 2.

In certain embodiments, the anti-CD28 antibody or antigen-binding fragment thereof comprises a HCDR1 region, a HCDR2 region, and a HCDR3 region that are at least about 90%, at least 91%, at least 92%, at least 93%, at least 94%, at least 95%, at least 96%, at least 97%, at least 98%, at least 99% or more identical to any one of the HCDR1, HCDR2, or HCDR3 amino acid sequences recited in Table 1.

In certain embodiments, the anti-CD28 antibody or antigen-binding fragment thereof is a chimeric or humanized antibody or antigen-binding fragment thereof.

In certain embodiments, the anti-CD28 antibody or antigen-binding fragment thereof is a monoclonal antibody or antigen-binding fragment thereof.

In certain embodiments, the anti-CD28 antibody or antigen-binding fragment thereof is a monospecific antibody.

In certain embodiments, the anti-CD28 antibody or antigen-binding fragment thereof is a bispecific antibody.

In certain embodiments, the anti-CD28 antibody or antigen-binding fragment thereof is a multispecific antibody. In an embodiment, the multispecific antibody comprises at least one Fab domain. In certain embodiments, the VH and VL domains of the Fab are replaced with any one of the VHH amino acid sequences recited in Table 2. The Fab domain may serve as a specific heterodimerization scaffold to which additional binding domains may be linked. Additional binding domains may be in several different formats, including, but not limited to, another Fab domain, a scFv, or an sdAb (e.g., VHH).

As used herein, “administer” or “administration” refers to the act of injecting or otherwise physically delivering a substance as it exists outside the body (e.g., an antibody provided herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases. The terms “cancer” and “tumor” encompass solid and hematological/lymphatic cancers and also encompass malignant, pre-malignant, and benign growth, such as dysplasia. Also, included in this definition are cells having abnormal proliferation that is not impeded (e.g., immune evasion and immune escape mechanisms) by the immune system (e.g., virus infected cells).

As used herein, “immune disease” refers to any disease which is associated with the development of an immune reaction in an individual, including a cellular and/or a humoral immune reaction. Examples of immune diseases include, but are not limited to, inflammation, allergy, autoimmune diseases, graft-related diseases, cancer, and viral infections.

As used herein, “autoimmune disease” refers to disease conditions and states wherein the immune response of an individual is directed against the individual's own constituents, resulting in an undesirable and often debilitating condition. As used herein, “autoimmune disease” is intended to further include autoimmune conditions, syndromes, and the like.

### Expression of Antigen-Binding Proteins

In one aspect, nucleic acid molecules encoding the antibodies and antigen-binding fragments thereof disclosed herein are provided. Methods of making binding proteins comprising expressing these nucleic acid molecules are also provided.

Nucleic acid molecules encoding the antibodies disclosed herein are typically inserted in an expression vector for introduction into host cells that may be used to produce the desired quantity of the antibodies. Accordingly, in certain aspects, the disclosure provides expression vectors comprising nucleic acid molecules disclosed herein and host cells comprising these vectors and nucleic acid molecules.

The term “vector” or “expression vector” is used herein to mean vectors used in accordance with the present disclosure as a vehicle for introducing into and expressing a desired gene in a cell. As known to those skilled in the art, such vectors may readily be selected from the group consisting of plasmids, phages, viruses and retroviruses. In general, vectors compatible with the disclosure will comprise a selection marker, appropriate restriction sites to facilitate cloning of the desired gene and the ability to enter and/or replicate in eukaryotic or prokaryotic cells.

Numerous expression vector systems may be employed for the purposes of this disclosure. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy chain constant region genes (e.g., human constant region genes) synthesized as discussed above.

In other embodiments, the antibodies may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antibody or fragment thereof has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, from supernatant of lysed cells culture, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HEK (human kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT® cells) (Biowa, Princeton, N.J.)). In one embodiment, NSO cells may be used. CHO cells are particularly useful. Host cell lines are typically available from commercial services, e.g., the American Tissue Culture Collection, or from authors of published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the antibodies featured in the disclosure can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the binding proteins can become part of inclusion bodies. In some embodiments, the binding proteins are then isolated, purified and assembled into functional molecules. In some embodiments, the binding proteins of the disclosure are expressed in a bacterial host cell. In some embodiments, the bacterial host cell is transformed with an expression vector comprising a nucleic acid molecule encoding a binding protein of the disclosure.

In addition to prokaryotes, eukaryotic microbes may also be used. Saccharomyces cerevisiae, or common baker's yeast, is the most commonly used among eukaryotic microbes, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Methods of Administering Antigen Binding Proteins

Methods of preparing and administering antigen binding proteins (e.g., the anti-CD28 antibody or antigen binding fragment thereof disclosed herein) to a subject are well known to or are readily determined by those skilled in the art. The route of administration of the antigen binding proteins of the current disclosure may be oral, parenteral, by inhalation or topical. The term parenteral as used herein includes intravenous, intraarterial, intraperitoneal, intramuscular, subcutaneous, rectal or vaginal administration. While all these forms of administration are clearly contemplated as being within the scope of the current disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the modified antibodies can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M or 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage, and should also be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. Isotonic agents, for example, sugars, polyalcohols, such as mannitol, sorbitol, or sodium chloride may also be included in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation typically include vacuum drying and freeze-drying, which yield a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in US20020102208 and U.S. Pat. No. 6,994,840, each of which is incorporated herein by reference. Such articles of manufacture can include labels or package inserts indicating that the associated compositions are useful for treating a subject suffering from, or predisposed to autoimmune or neoplastic disorders.

Effective doses of the compositions of the present disclosure, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

As previously discussed, the antigen binding proteins of the present disclosure, immunoreactive fragments or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the modified antigen binding proteins, immunoreactive fragment or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the antigen binding proteins of the disclosure may be administered to a human or other animal in accordance with the aforementioned methods of treatment in an amount sufficient to produce a therapeutic or prophylactic effect. The antigen binding proteins of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The biological activity of the pharmaceutical compositions defined herein can be determined for instance by cytotoxicity assays, as described in the following examples, in WO 99/54440 or by Schlereth et al. (Cancer Immunol. Immunother. 55 (2006), 503-514). The biological activity can also be determined by T cell activation assays, such as through the detection of pro- or anti-inflammatory cytokine expression. “Efficacy” or “in vivo efficacy” as used herein refers to the response to therapy by the pharmaceutical composition of the invention, using e.g. standardized NCI response criteria. The success or in vivo efficacy of the therapy using a pharmaceutical composition of the invention refers to the effectiveness of the composition for its intended purpose, i.e. the ability of the composition to cause its desired effect, i.e. depletion of pathologic cells, e.g. tumor cells or repression of active immune cells. The in vivo efficacy may be monitored by established standard methods for the respective disease entities including, but not limited to white blood cell counts, differentials, Fluorescence Activated Cell Sorting, bone marrow aspiration. In addition, various disease specific clinical chemistry parameters and other established standard methods may be used.

### EXAMPLES

The following examples are put forth so as to provide those of ordinary skill in the art with a complete disclosure and description of how to make and use the methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celsius, and pressure is at or near atmospheric.

### Example 1

### Materials and Methods

### Cell Lines and Human Donor-Derived Cells

Buffy coats from healthy donors were obtained from a French blood bank (Etablissement Frangais du Sang). After purification of peripheral blood mononuclear cells with Ficoll gradient, total CD3⁺ T cells were enriched using the RoboSep™ Human T-Cell Enrichment Kit (StemCell Technologies, 19051) according to the manufacturer's instructions. Dendritic cells (DCs) were generated by culturing monocytes isolated from peripheral blood mononuclear cells using a monocyte purification kit (StemCell Technologies, 17858) in vitro for 7 days with 200 ng/mL IL-4 (Miltenyi Biotec, 130-093-922) and 200 ng/mL granulocyte macrophage colony-stimulating factor (Miltenyi Biotec, 130-093-866). FreeStyle™ HEK293-FS cells were purchased from Invitrogen, Jurkat cells from the American Type Culture Collection (ATCC, TIB-152), and Jurkat-IL-2-Luc2P cells from Promega. All cell lines were grown at 37° C. in a humidified atmosphere containing 5% CO₂ in media recommended by the cell supplier. All cell line culture media, and reagents were purchased from Gibco. TGN1412 and 9.3 antibodies were produced and purified internally.

### Transfection for CD28 Expression

FreeStyle™ HEK293-FS cells were transfected with high-quality plasmid preparations encoding human CD28 using 293fectin™ (Gibco, 12347-019) according to the manufacturer's instructions. CD28 expression was evaluated 48 hours after transfection with flow cytometry using AF647-conjugated anti-human CD28 mAb (BD Pharmingen™, 560683).

### Example 2

### Llama Immunization and Library Construction

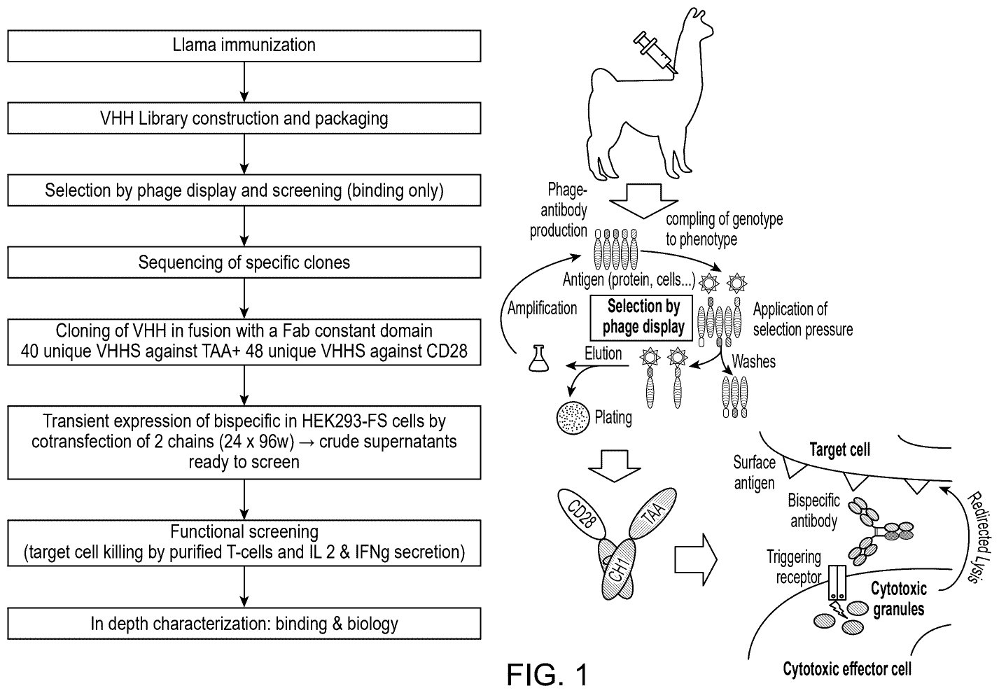

Immunization and library construction were conducted by the VIB Nanobody Service Facility (Brussels, Belgium). As shown in FIG. 1, a llama was immunized for CD28 and TAA (DNA immunization) by intradermal injections four times, each time with approximately 2-mg vector harboring the genes of interest (human full-length CD28 and human full-length TAA). After each injection, the animal was electroporated to introduce the DNA constructs into animal cells. Three weeks after the last DNA injection, the animal was subcutaneously boosted with recombinant proteins (human CD28 [Sino Biological, 11524-HCCH], human TAA produced internally; adjuvant for protein boost, GERBU LQ 3000). Four days after the protein boost, anticoagulated blood was collected for VHH library construction as shown in FIG. 1. FIG. 1 summarizes the general workflow for CD28xTAA bispecific screening: a llama was immunized for CD28 and TAA (DNA immunization), and a VHH library was constructed for phage display selections on recombinant protein and cells; clones were rapidly screened for binding, sequenced, and cloned in frame with a human constant domain CH1 or Ck to produce bsFabs by transient transfection in FreeStyle™ HEK293-FS cells. Cell supernatants were screened functionally in a primary cell assay, and clones of interest were further characterized for their binding properties and biological function.

The VHH library was constructed for phage display selections on recombinant protein and cells as previously described. Total ribonucleic acid was extracted from peripheral blood lymphocytes and used as a template for first strand complementary DNA synthesis with oligo(deoxythymine) primers. VHH-encoding sequences were amplified by polymerase chain reaction from complementary DNA, digested with PstI and NotI, and cloned between the PstI and NotI sites of the phagemid vector pHEN4 upstream of the human influenza hemagglutinin decapeptide tag. A VHH library of approximately 108 independent transformants was obtained. As shown in FIG. 1, clones were then rapidly screened for binding, sequenced, and cloned in frame with a human constant domain CH1 or Ck to produce bsFabs by transient transfection in FreeStyle™ HEK293-FS cells. Cell supernatants were screened functionally in a primary cell assay, and clones of interest were further characterized for their binding properties and biological function.

### Phage Display Panning

A ready-to-use phage-VHH preparation was obtained as described. The bacteria library was grown in a 2YTAG medium (2×YT medium, ampicillin 100 μg/mL, glucose 2%) until the absorbance at 600 nm (OD600) reached 0.5 and infected with M13K07 helper phage (Invitrogen). After centrifugation, bacteria were resuspended in a 2YTAK medium (2×YT medium, ampicillin 100 μg/mL, kanamycin 50 μg/mL) and grown overnight. Phage particles were precipitated from culture supernatant by addition of polyethylene glycol 8000 (PEG8000) 20% (weight/volume [w/v]) and 2.5 M NaCl, centrifuged, and resuspended in phosphate-buffered saline (PBS). Phages were subjected to one more wash and precipitation step and finally resuspended in cold PBS/glycerol 15% (volume/volume [v/v]).

### Panning on Recombinant Protein

Panning on recombinant protein was performed as described. M-450 Epoxy beads (Dynabeads, Invitrogen) were coated with His-tagged CD28 or TAA-recombinant proteins according to the manufacturer's recommendations. The phage-VHH library (10¹¹ phages/selection round) and beads (coated and naked) were saturated in PBS/milk 2% (w/v) for 1 hour at room temperature (RT). The phage-VHH library was first depleted twice by 30-minute incubation on naked beads to eliminate nonspecific clones. Unbound phage-VHHs were recovered and incubated with target-coupled beads in PBS/milk 2% (w/v) for 2 hours at RT. After 10 washes with PBS/Tween 0.1% (v/v) and two washes with PBS, bound phage-VHHs were resuspended in PBS (output selection), added to exponentially growing TG1 bacteria, and either amplified overnight in 2YTAG medium for a new round of panning or plated on 2YTAG plates.

### Panning on Cells

Panning was performed at 4° C. on CD28- or TAA-transfected FreeStyle™ HEK293-FS cells. After two washes with PBS, the cell pellet was resuspended in PBS and loaded on a fetal bovine serum/Percoll gradient as previously described. After centrifugation, the cell layer was collected and washed twice with PBS. Recovered cells with bound phages were added into a second fetal bovine serum/Percoll gradient and washed before mechanical lysis using beads (Dynabeads, Invitrogen). Recovered phage-VHHs were used to infect exponentially growing Escherichia coli TG1 bacteria and either amplified overnight in 2YTAG medium for a new round of panning or plated on 2YTAG plates.

### Fab-Like Construction, Production, and Purification

After amplification with polymerase chain reaction, complementary DNA of the anti-CD28 VHH or anti-TAA VHH or anti-foot-and-mouth disease virus (FMDV) VHH (Harmsen et al. Veterinary microbiology. 2007; 120(3-4):193-206) were cloned into a proprietary mammalian expression vector in frame with either the human CL domain or the human IgG1 CH1 domain fused to human influenza hemagglutinin and 6-His tags. Plasmids were purified using NucleoBond Macherey-Nagel kits and Sanger sequenced. Bispecific (bsFab) or bivalent Fab-like (bvFab) antibodies were produced by cotransfecting FreeStyle™ HEK293-FS cells with a mix of two plasmids encoding two distinct (bsFab) or two identical (bvFab) VHHs fused to each of the Fab constant domains. Supernatants were harvested 7 days later, purified on Nickel affinity columns, and analyzed on CALIPER GXII (Perkin Elmer).

### Flow Cytometry Binding and Competition Assays

All flow cytometry assays were performed on a MACSQuant cytometer (Miltenyi Biotec, Germany) using V-bottom 96-well microtiter plates. Cells were gated on viable single cells (Dapi staining), and 10⁴ events were collected for each sample. Data were analyzed using the MACSQuant software, and the results were expressed as a median of fluorescence intensity.

Jurkat cells were first incubated with serial dilutions of bvFabs for 1 hour at 4° C. and then with a human CD80-Fc fusion at its effective concentration at 90% (EC90) for 30 minutes at 4° C. Bound ligands were detected with an anti-human IgG (Fc-specific) mAb (Sigma, 12136) followed by Alexa647-conjugated goat anti-mouse mAb (Invitrogen, A11013).

### Competitive Binding Assays (TGN1412 and 9.3 mAb Competition Using Enzyme-Linked Immunosorbent Assay [ELISA])

### Phage-VHH Production in 96-Well Plates

Individual TG1 colonies of CD28 VHHs of interest were grown in 2YTA medium at 37° C. until OD600 reached 0.5. Cells were then infected with the M13K07 helper phage and grown overnight in 2YTAK at 30° C. Supernatants containing phage-VHHs were harvested and used for testing.

### ELISA

ELISA was performed on Nunc® MaxiSorp™ 96-well plates (Sigma) precoated overnight with 1 μg/mL of human CD28-recombinant protein in PBS at 4° C. and further saturated with PBS/milk 2% (w/v) for 1 hour at RT. A serial dilution of competitor mAbs (TGN1412 or 9.3) was then incubated for 1 hour at RT and phage-VHH at its EC90 was further added for 30 minutes at RT. After several washes in PBS/Tween 0.1% (v/v), the anti-M13 horseradish peroxidase-conjugated mAb (Santa Cruz Biotechnology, sc-53004) was added to detect bound phage-VHHs. Detection of peroxidase activity was performed using 3,3′,5,5′-tetramethylbenzidine substrate (Thermo Scientific, 34029). Absorption was measured at OD 450 nm on a SpectraMax microplate reader (Molecular Devices) after addition of sulfuric acid stop solution.

### Reporter Assay

For Condition with CD3 Preactivation

Wells were coated (plate Costar 3917) with 50 μL of anti-CD3 (UCHT-1 clone, BioLegend, BLE300414), stored at 4° C. overnight, and then washed twice with 100-μL PBS/well. Jurkat-IL-2-Luc2P cells were harvested during their exponential growth phase, and 25 μL of the cell suspension was added into a 96-well plate (50,000 cells/well) with 25 μL of test compound.

### For Cross-Linking Experiments

Test compounds were preincubated with a saturating concentration of anti-human Fab (Sigma, 15260) or anti-human Fc (Sigma, 12136) for 30 minutes at RT. Jurkat-IL-2-Luc2P cells were harvested during their exponential growth phase, and 25 μL of the cell suspension was added into a 96-well plate (50,000 cells/well) with 25 μL of cross-linked or non-cross-linked test compounds.

For Condition with TAA-Expressing Cells

Jurkat-IL-2-Luc2P cells were harvested during their exponential growth phase and mixed with TAA-expressing cells to obtain a final ratio of 1:1 between reporter and accessory cells. 25 μL of the cell suspension was added into a 96-well plate (50,000 cells/well) with 25 μL of test compounds.

For all three conditions (CD3 preactivation, cross-linking, and with TAA-expressing cells), the plate was incubated for 6 hours in a humidified incubator at 37° C. with 5% CO₂. 50 μL of Bio-Glo™ (Promega, G7941) reagent prepared according to the manufacturer's instructions was then added to each well and mixed. At least 5 minutes were allowed for complete cell lysis to occur, following which luminescence was measured using the Envision multimode plate reader (Perkin Elmer).

### T-Cell Activation Assay

U-bottom 384-well plates were coated with 5 μg/mL anti-human CD3 antibody (eBioscience, 15288347, OKT3 clone) overnight at 4° C. Plates were washed with PBS and 50,000 T cells were added in complete X-VIVO™ 15 culture media (Lonza, BE02-060F) in the presence of 10, 30, and 100 nM of negative (isotype or FMDV bvFab) and positive (TGN1412, or 9.3 mAbs) control antibodies or test compounds (CD28xFMDV bsFabs and CD28 bvFabs) and incubated at 37° C. in a 5% CO₂ incubator. After 6 days of incubation, supernatants were collected and stored at −20° C. until cytokine measurement. Promega CellTiter-Glo® reagent was added to the cells for cell counting. Cytokine levels were measured using a Homogeneous Time Resolved Fluorescence human IFNγ/tumor necrosis factor (TNF)α cytokine kit according to the manufacturer's instructions (Cisbio). Samples were read on the PHERAstar FSX multimode reader (BMG Labtech). Data were expressed as percentage of effect compared with negative control.

### Mixed Leukocyte Reaction (MLR)

CarboxyFluorescein Succinimidyl Ester (CFSE)-labeled CD3⁺ T cells (1×10⁵) and allogeneic DCs (1×10⁴) were cocultured with or without 10 nM of negative (FMDV bvFab) and positive (TGN1412 or 9.3 mAbs) control antibodies or test compounds (CD28xTAA bsFabs and CD28 bvFabs) at the initiation of the assay. After 4 days, supernatants were collected, and cytokine levels measured using a CBA human Th1/Th2/Th17 kit (BD Biosciences, 550749) according to the manufacturer's instructions. Cells were stained with an antibody cocktail for CD4 (BD Biosciences, 563550), CD8 (BD Biosciences, 560662), CD25 (BD Biosciences, 555434), and CD69 (BD Biosciences, 562617). T-cell proliferation was measured using CFSE dilution. Samples were analyzed on a Fortessa X-20 flow cytometer (BD Biosciences).

### Epitope Binning

Tandem epitope binning of CD28 antibodies was done with surface plasmon resonance (SPR) using a BIAcore T200 (upgraded T100, Cytiva Life Sciences, France) instrument with HBS EP+ as running buffer (0.01 M HEPES pH 7.4, 0.15 M NaCl, 3 mM ethylenediamine tetraacetic acid, 0.005% [v/v] surfactant P20; Cytiva Life Sciences Biacore BR100826). An anti-human Fc antibody (Human Antibody Capture Kit, Cytiva LifeSciences BR-1008-39) was covalently coupled on Sensor Chip CM5 (Cytiva LifeSciences, Biacore BR100530).

Each of the four flow cells was prepared independently. All four flow cells were first activated for 7 minutes with 1-ethyl-3-(3-dimethylaminopropyl) carbodiimide/N-hydroxysuccinimide mixture (75-11.5 mg/mL) using a flow rate of 5 μL/minute (Amine Coupling Kit, Cytiva LifeSciences, BR100050). The anti-human Fc antibody was diluted to 25 g/mL in 10 mM acetate pH 5.0 and coupled for 7 minutes using a flow rate of 5 μL/minute. Uncoupled sites were then deactivated with a 7-minute pulse of 1 M methanolamine pH 8.5 using a flow rate of 5 μL/minute. Approximately 10,000 resonance units (RU) of antibodies were typically observed on all spots.

Following surface preparation, huCD28-huFc (Sino Biological, 11524-H02H) was diluted to 0.5 μg/mL in running buffer and captured for 120 seconds at 5 L/minute up to 200 RU (only on FC2 or FC4). Dual injection was performed with a 30 μL/minute first injection of crude HEK293-FS supernatant containing the first test bsFab for 120 seconds (saturation and stability conditions) followed by a second injection of crude HEK293-FS supernatant containing the second test bsFab for 60 seconds (on both FC1-FC2 or FC3-FC4). Dissociation was then monitored for 60 seconds. Surfaces were regenerated with a 30-second pulse of 3M MgCl₂ using a flow rate of 10 μL/minute.

Sensorgrams were double referenced by subtracting a reference spot (without huCD28-huFc) and a buffer injection. Normalized competition signal (C=normalized signal SN2-normalized signal SN1) was calculated for each antibody pair. Normalized signal corresponds to the signal normalized with the huCD28-Fc capture. For qualitative data, C<+5 represented no binding of SN2 and competition; C>+5 represented binding of SN2 and no competition. For quantitative data (full or partial competition) based on normalized signals, the equation C*100/(theoretical NormB SN2−NormB dissociation SN1) was used for analysis.

### Statistical Analysis

A two-way hierarchical clustering using Euclidian distance matrix and Ward's aggregation method was performed with statistical software R 3.2.3 to regroup binders with the same profiles in cluster when each binder was injected first or second. The number of the cluster was selected using a graphical representation of the number of classes in function of the inertia jump of the dendrogram (where the inertia is a variance criterion). The p-value for Pearson correlation coefficient was calculated using a two-tailed t-test.

### Example 3

### Results

### Selection, Screening, and Reformatting of VHH

The current study aimed to explore the use of VHH-based bsFabs in activating T cells via the CD28 receptor to kill tumor cells and provide an in-depth characterization of a panel of CD28 constructs in terms of binding, epitope diversity, agonist and antagonist properties.

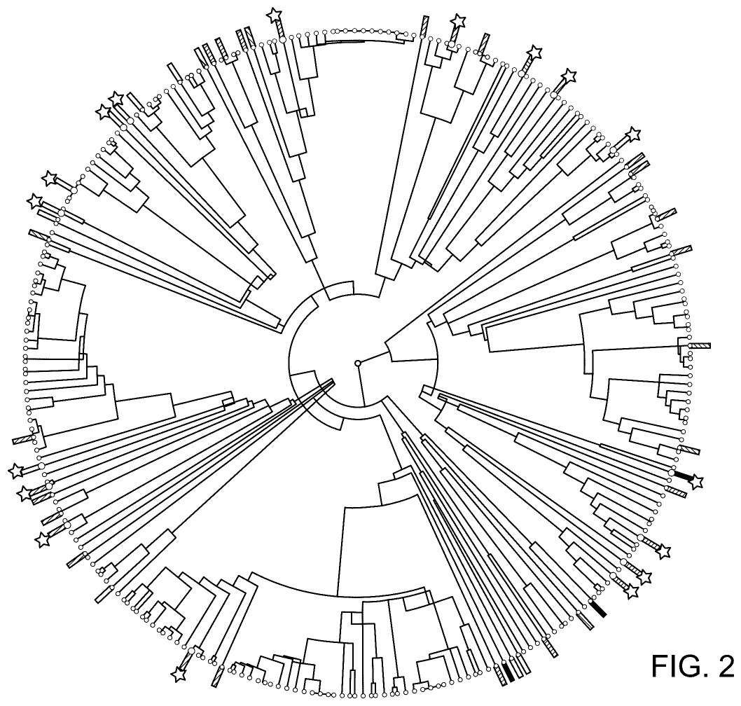

VHHs against CD28 and TAA were isolated using phage display. A large panel of highly diverse VHH sequences library was constructed and biopannings were performed in two rounds of selection on the purified recombinant ectodomain of each antigen or target-expressing cells. After selection, one plate (95 clones) was screened for each round using ELISA on recombinant antigens (data not shown). For each selection, more than 80% of the tested clones were specific for their target. All specific clones were further sequenced, and the sequence analysis revealed high diversity and low redundancy in isolated CD28 VHHs as shown in FIG. 2. In short, sequence analysis consisted of aligning VHH sequences of all hits identified by phage display selections by Clustal Omega; the sequence tree shown in FIG. 2 was then constructed using the guided-tree output obtained with Clustal Omega. The scale bar in the figure corresponds to the distance between sequences, the rectangular tags represent the 48 VHHs tested in the epitope binning with a color code per bin (blue=D, orange=D*, green=E, red=F, gray=not determined), and the black stars highlight compounds 2 to 18 that were further tested in the cellular assays. Non-statistical analysis (binning with second injection) clearly identified a subgroup in the D group or 16 subgroups; however, no statistically significant difference was observed.

The cluster diversity analysis based on a sequence identity of 95% revealed that among 277 CD28-specific clones, 66 clusters of VHHs were isolated on recombinant protein and 56 on CD28-transfected cells. Similar results were found for TAA-specific VHHs (data not shown).

Forty-seven anti-CD28 VHHs with high sequence diversity (colored tags in FIG. 2) were subsequently cloned in fusion with the C lambda IgG domain, and 39 anti-TAA VHHs were cloned in fusion with the CH1 IgG domain for eukaryotic expression.

### Example 4

Production and Functional Screening of CD28xTAA bsFabs

CD28xTAA bsFabs were generated using the compact and linker free Fab-like format depicted in FIG. 1 (33). More precisely, a matrix combination of 47 anti-CD28 (plus 1 negative control)×39 anti-TAA (plus 1 negative control) bsFabs (i.e., twenty-four 96-well plates) was produced at the 1-mL scale. Quality control was done on supernatants of 288 randomly selected wells across the twenty-four 96-well plates for a total of three quality control plates. Quality control consisted of the following three components: sufficient antibody quantity was confirmed by means of titration antibody production in HEK293-FS supernatant (around 20 μg/mL); caliper was performed to confirm the presence of dimers in the supernatant; and ELISA-binding capabilities of the Fab-like antibodies produced in HEK293-FS supernatant on the two targets CD28 and TAA were analyzed to reveal efficient binding in almost all of the 288 tested antibodies.

The supernatants of the twenty-four 96-well plates of bsFabs were then tested in a functional assay evaluating the capacity of bsFabs to trigger killing of TAA-expressing tumor cells by purified T cells as well as IL-2 and IFNg secretion at 72 hours. None of the 1800 bsFabs showed tumor cell killing and IFNg secretion (data not shown) despite experiment validation with expected results obtained with positive (CD3xTAA) and negative (CD3×irrelevant target and CD28×irrelevant target) control bsAbs. A duplicate experiment with one-quarter of the constructs confirmed the initial results. Finally, a set of 34 bsFabs was purified and tested in the same assay in a dose range experiment (0.1-300 nM) (data not shown). No tumor cell killing and IFNg secretion were observed even at the highest tested dose of 300 nM. A slight IL-2 secretion was detected for some clones at the highest doses.

### Example 5

### In-Depth Characterization of Anti-CD28 VHHs

### Epitope Diversity

To test the hypothesis that the absence of killing properties of all tested bsFabs might be due to lack of diversity in the epitope and function of the selected CD28 VHH, the epitope diversity of the anti-CD28 VHH was first characterized.

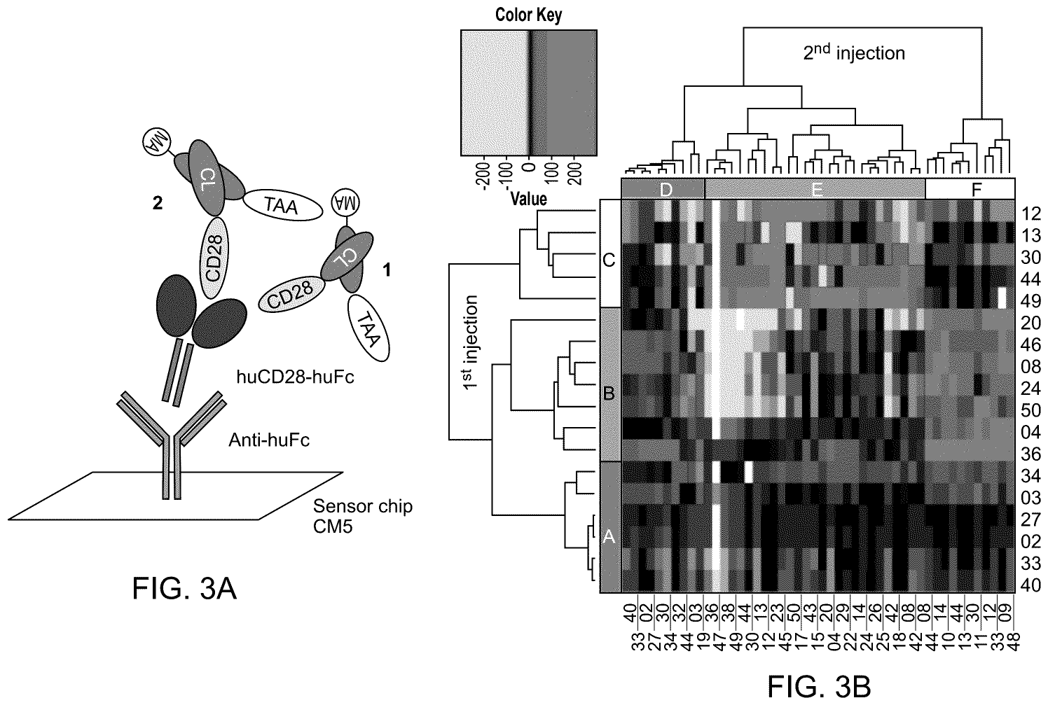

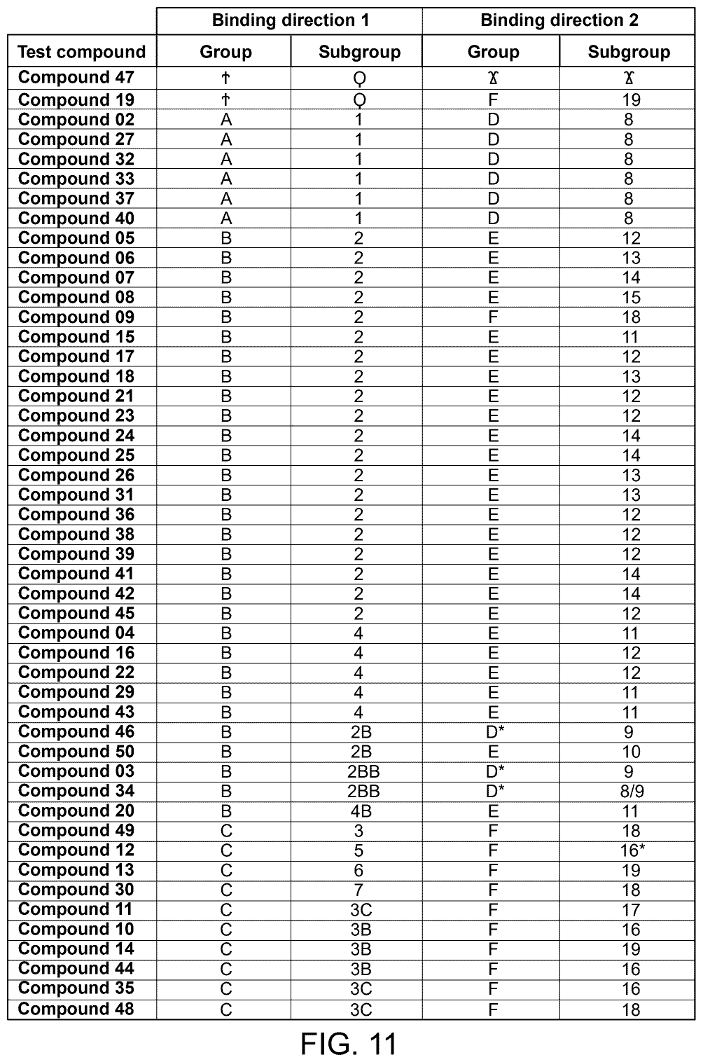

An epitope binning experiment using SPR on 48 CD28 VHHs (including the 47 tested above) revealed three statistically significant epitope clusters (two way hierarchical clustering using Euclidian distance matrix and Ward's aggregation method) that could be further refined to six subclusters as shown in FIG. 3 and in the Table in FIG. 11. Shortly, FIG. 3 shows epitope binning of CD28 bsFabs, i.e. the competition between anti-CD28 VHH with SPR. FIG. 3A depicts the following experimental scheme: human CD28-Fc was captured on Sensor Chip CM5 using an anti-human Fc antibody, subsequently, the first CD28xTAA bsFab was added, followed by a second one for binning analysis, and finally 48×48 constructs were tested. FIG. 3B depicts the competition data that were collected in a heatmap indicating whether or not each VHH pair competes (green indicates blocked and red, not blocked). The heatmap was processed using two-way hierarchical clustering generating a dendrogram and 3 significantly different epitope clusters (A, B, C and D, E and F, depending on injection sequence). Asymmetry dependent on injection sequence was observed as shown in FIG. 3B.

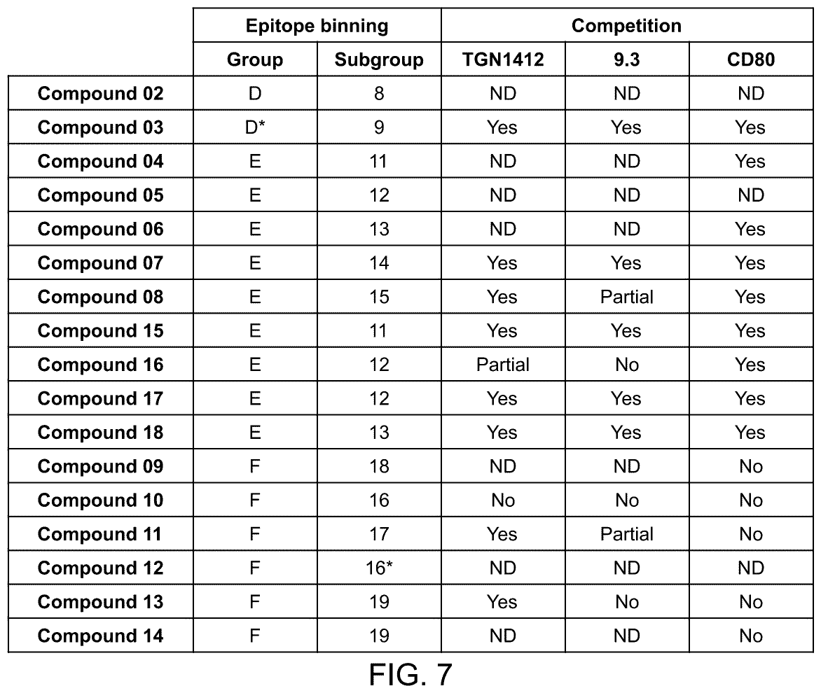

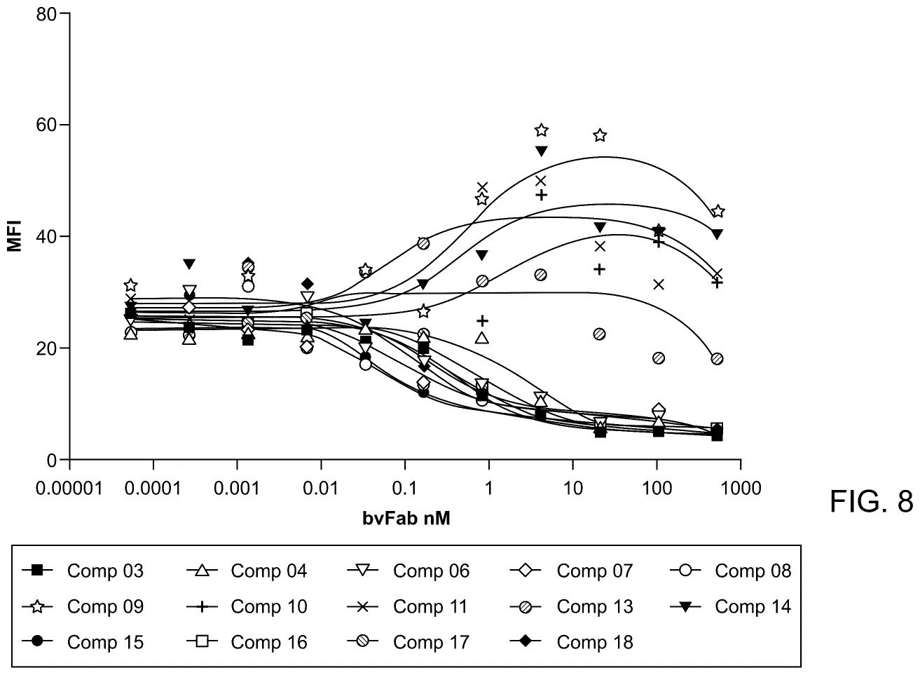

A limited set of 17 CD28 VHHs (compounds 2-18, identified by stars in FIG. 2) spanning the different epitope bins and maximizing sequence diversity was selected to perform an orthogonal analysis of epitope diversity in fluorescence-activated cell sorting competition assays with CD80 or in ELISA competition assays with TGN1412 or 9.3 mAbs. This analysis revealed that CD28 VHHs can be classified as competitors and non-competitors of CD80 shown in FIG. 8, which depicts the competition of CD28 bvFabs with CD80 using flow cytometry, and independently as full, partial, or non-competitors of TGN1412 or 9.3 mAbs. In summary, FIG. 8 depicts serial dilutions of CD28 bvFabs incubated with Jurkat cells before addition of recombinant human CD80-Fc protein at its EC90. Ligand binding was detected via flow cytometry using Alexa 647-conjugated anti-human Fc mAbs. The results are expressed as median of fluorescence intensity (MFI). Epitope binning and all competition data of the VHH subsets are reported in the table depicted in FIG. 7, which is a summary table of results from epitope binning and competition with TGN1412 and 9.3 benchmark antibodies or CD80, one of the natural ligands of CD28.

### Example 6

### Agonist Properties: Reporter Assays

To evaluate the agonist capacity of the CD28 compounds, a bioluminescent reporter cell-based assay was used, which comprised a genetically engineered Jurkat T-cell line that expresses a luciferase reporter gene driven by an IL-2 promoter.

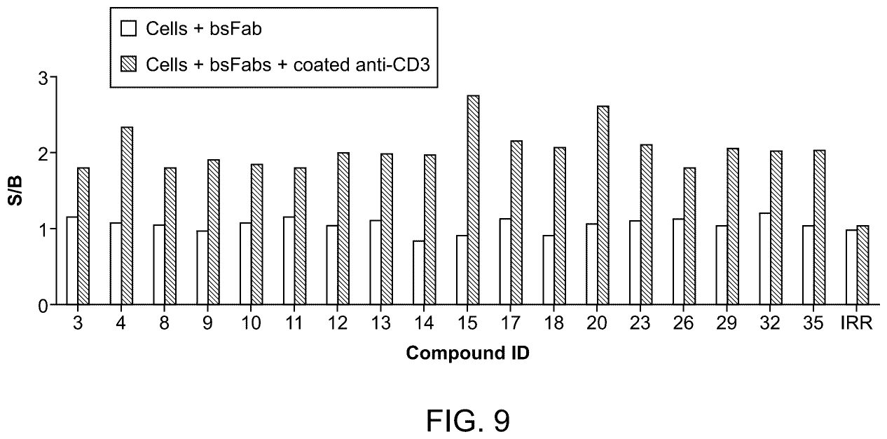

A series of CD28xTAA bsFabs were first tested in the presence of TAA-expressing HCT116 cells with and without suboptimal TCR activation of engineered Jurkat cells through anti-CD3 coated mAb. All bsFabs allowed the activation of the IL-2 promoter if TCR was preactivated in FIG. 9, which implies that bsFabs were able to bind simultaneously to two cells, with each cell expressing either CD28 or TAA. FIG. 9 shows that CD28xTAA bsFabs can simultaneously bind two cells expressing either CD28 or TAA. In summary, Jurkat-IL-2-Luc2P cells and TAA-expressing HCT116 cells at a 1:1 ratio were added to 96-well microplates precoated or not with anti-CD3 mAb. The cells were then incubated with test compounds at 30 nM for 6 hours at 37° C. before luminescence was measured. The luciferase luminescence signal normalized to cells treated with negative control (IRR=FMDV bvFab) is shown (S/B=Signal to Background). Each bar on the graph in FIG. 9 represents the mean value of two independent experiments.

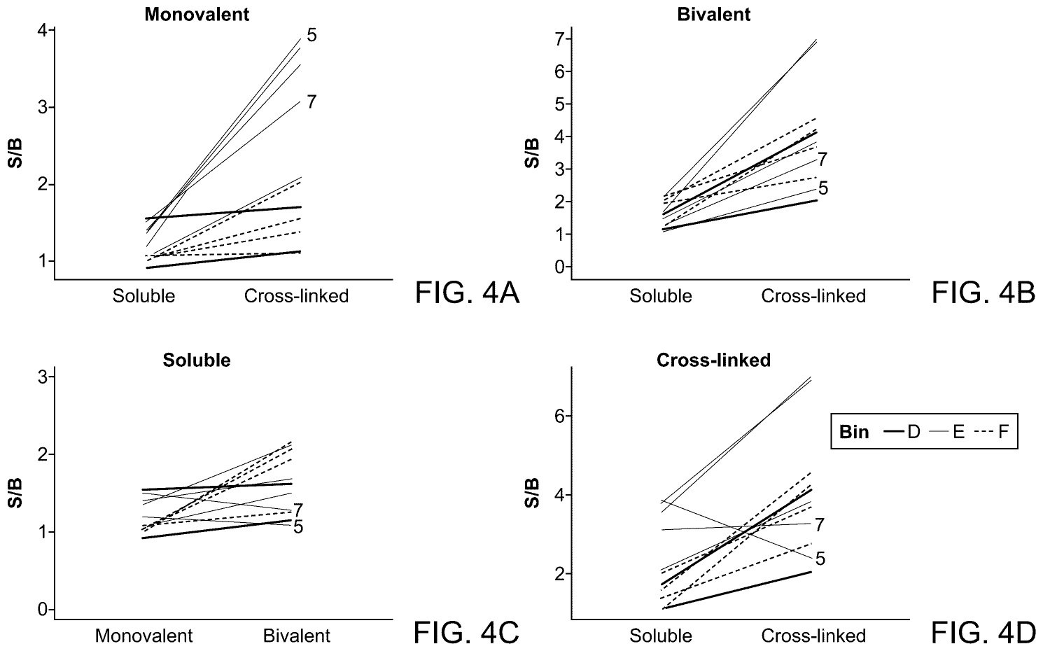

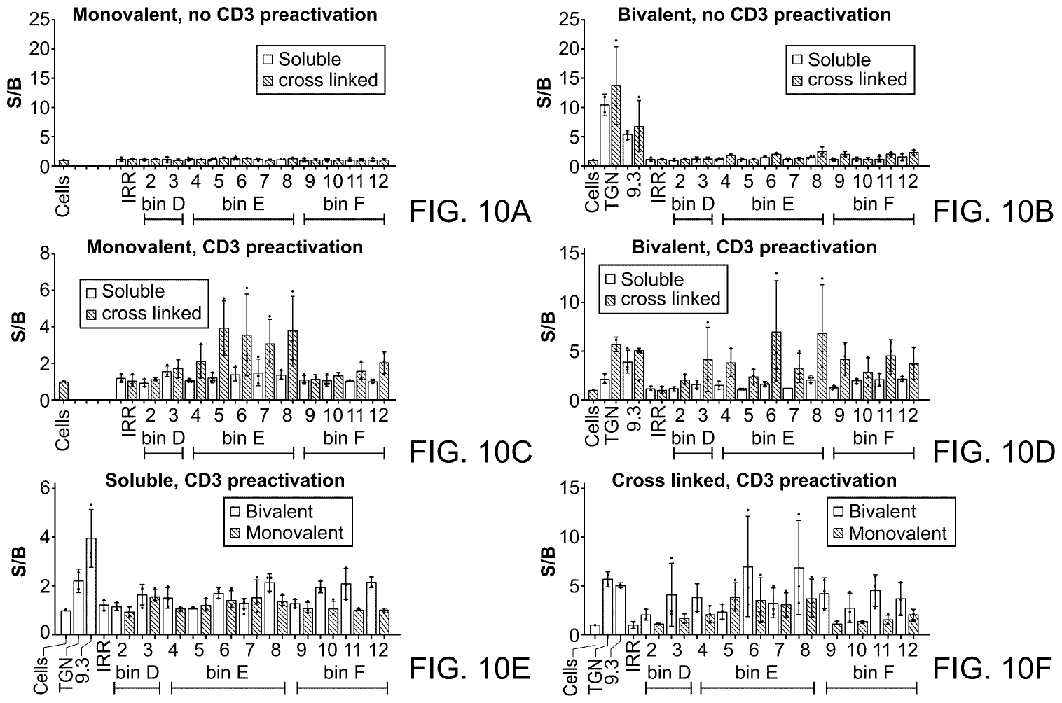

Eleven CD28 VHHs warranting maximal epitope coverage and sequence diversity (compounds 2-12 of table in FIG. 7) were retained for in-depth functional characterization. They were produced and purified in the Fab-like format, either as CD28xFMDV bsFab (monovalent on CD28) or CD28 bvFab (bivalent Fab-like, where the same VHH is fused with both CH1 and CL). Evaluation of these bsFabs and bvFabs without suboptimal TCR activation through anti-CD3-coated mAb revealed that in the absence of cross-linking none of the constructs regardless of valency were active, while TGN1412 and 9.3 mAbs were highly active as shown in FIGS. 10A and 10B. FIGS. 4 and 10 show the evaluation of CD28 bsFabs and bvFabs in the following IL-2 luciferase reporter assay: Jurkat-IL-2-Luc2P cells were added to 96-well microplates precoated with anti-CD3 mAb as shown in FIG. 4 and FIGS. 10 C to F, or not as shown in FIGS. 10 A and B; the cells were then incubated with previously cross-linked or non-cross-linked bsFabs and bvFabs at 100 nM for 6 hours at 37° C. before luminescence was measured, and the luciferase luminescence signal normalized to cells treated with negative control (IRR=FMDV bvFab) is shown (S/B=Signal to Background). These assays were performed three times and plotted in FIGS. 4 and 10.

However, when cross-linked, some bvFabs of bins E and F were active at levels lower than those seen for TGN1412 and 9.3 mAbs as shown in FIG. 10B. The capacity of the 9.3 mAb to induce luciferase expression without CD3 coactivation was unexpected with respect to its description as an agonist and not a superagonist. It suggests some discrepancy between T-cell activation and the Jurkat reporter assay.

With CD3 coactivation, in the absence of cross-linking, one bvFab from bin E and three of four bvFabs from bin F were able to induce IL-2 pathway activation, while all other bsFabs and bvFabs were inactive in soluble conditions as shown in FIG. 4C, which shows monovalent CD28xFMDV bsFab versus bivalent CD28 bvFab without cross-linking, and FIG. 10E, which shows monovalent CD28xFMDV bsFab versus bivalent CD28 bvFab without cross-linking after CD3 preactivation.

When cross-linked, the IL-2 pathway was induced for all bvFabs as shown in FIG. 4B showing bivalent CD28 bvFab with and without cross-linking and FIG. 10D showing bivalent CD28 bvFab with and without cross-linking after CD3 preactivation, all bin E bsFabs, and one bin F bsFab as shown in FIG. 4A showing monovalent CD28xFMDV bsFab with and without cross-linking. and FIG. 10C showing monovalent CD28xFMDV bsFab with and without cross-linking after CD3 preactivation. Superior activity for most CD28 clones under bvFab over the bsFab format was observed, revealing maximal impact on IL-2 induction when combining cross-linking with bivalency as shown in FIG. 4B showing bivalent CD28 bvFab with and without cross-linking and 4D showing monovalent CD28xFMDV bsFab versus bivalent CD28 bvFab with cross-linking, and FIG. 10D showing bivalent CD28 bvFab with and without cross-linking after CD3 preactivation and 10F showing monovalent CD28xFMDV bsFab versus bivalent CD28 bvFab with cross-linking after CD3 preactivation

Clones 5 and 7 from bin E revealed a unique behavior in that their cross-linking allowed CD28 activation as shown in the plots in FIGS. 4A, showing monovalent CD28xFMDV bsFab with and without cross-linking, and 4B, showing bivalent CD28 bvFab with and without cross-linking, while increase in valency did not, as shown in FIG. 4C showing monovalent CD28xFMDV bsFab versus bivalent CD28 bvFab without cross-linking, and 4D showing monovalent CD28xFMDV bsFab versus bivalent CD28 bvFab with cross-linking. In both bsFab and bvFab formats, these 2 clones were inactive and active in the absence and presence of cross-linking, respectively; however, bvFabs were not more active than bsFabs.

### Agonist Properties: T-Cell Activation

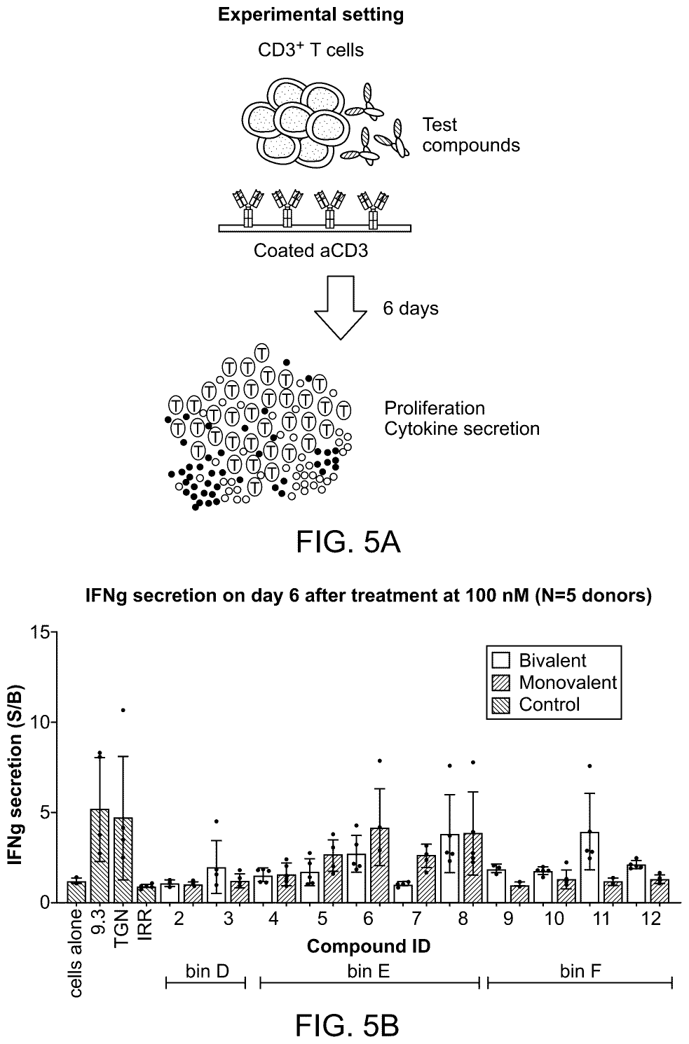

Agonist properties of the constructs were then evaluated using a primary T-cell activation assay where compounds were tested in soluble conditions without cross-linking with suboptimal TCR activation through anti-CD3-coated mAb. Some but not all CD28 bvFabs showed the capacity to increase IFNg secretion as shown in FIG. 5. FIG. 5 shows the evaluation of CD28 bsFabs and bvFabs in a T-cell activation assay. FIG. 5A shows the following experimental setting: human purified CD3+ T cells were added to 384-well microplates pre-coated with anti-CD3 mAb in the presence of 10, 30, and 100 nM of negative (IRR=FMDV bvFab) and positive (TGN1412 and 9.3 mAbs) control antibodies or test compounds (CD28xFMDV bsFabs and CD28 bvFabs). After 6 days of incubation, IFNγ and TNFα levels in cell culture supernatant were determined and T cells were counted. (B) IFNγ secretion on day 6 after treatment at 100 nM. Data are expressed as the ratio of IFNγ level in treated cells to cells treated with IRR negative control. Each bar represents the mean±standard error of the mean of 5 independent assays (5 independent donors). A strong correlation was observed between IFNg secretion and the two other readouts, namely T-cell proliferation (r=0.63 and p-value=8.10-41) and TNFα secretion (r=0.87 and p-value=7.8.10-110) with some inter-donor variation (data not shown).

Bin D was inactive in monovalent and bivalent formats. In addition, four of five bin E constructs were active in the monovalent bsFab format (compounds 5-8); two were active in the bivalent format (compounds 6 and 8). These findings were not consistent with the reporter assay. All bin F monovalent bsFabs were inactive, while all bivalent constructs were agonists (ranging in strength). These findings were consistent with those of the reporter assay.

T-cell activation assay conditions were comparable with some of those of the reporter assay as shown by bsFab/bvFabs in FIG. 4C; however, the correlation between T-cell activation and the Jurkat reporter assay was not strong. Most bsFabs from bin E showed unexpected T-cell activation despite monovalency and the absence of cross-linking.

### Antagonist Properties

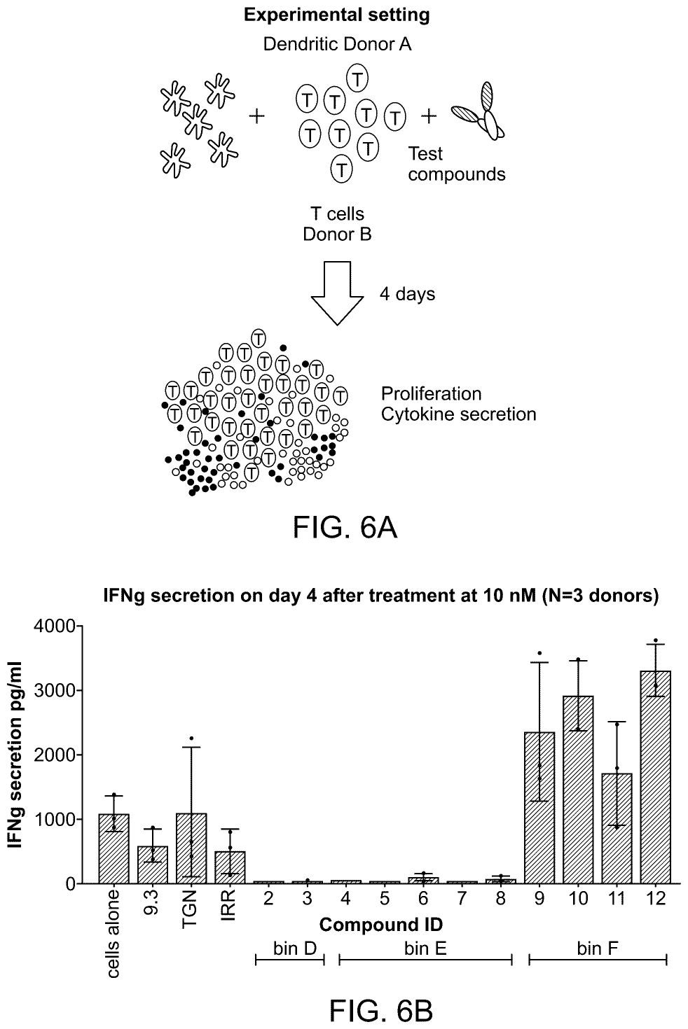

The CD28 bsFabs and bvFabs were evaluated in an MLR assay as shown in FIG. 6. This is a two-cell system where monocyte derived DCs known to express CD28 ligands (CD80/86) from a first donor, trigger TCR activation of T cells from a second donor. FIG. 6A shows the experimental setting CFSE-labeled CD3+ T cells and allogeneic monocyte derived DCs were cocultured with or without 10 and 100 nM of negative (IRR=FMDV bvFab) and positive (TGN1412 and 9.3 mAbs) controls or test compounds (CD28 bvFabs) at the initiation of the assay. After 4 days, cytokine levels in cell culture supernatant and T-cell proliferation were determined. FIG. 6B shows IFNg secretion on day 4 after treatment at 10 nM. Each bar represents the mean value±SEM of 3 independent assays (3 independent donors).

All bvFabs from bin F increased T-cell proliferation (data not shown) and cytokine secretion shown in FIG. 6 and were more active than TGN1412 and 9.3 mAbs. Figure All other bvFabs blocked T-cell proliferation and cytokine secretion as shown in FIG. 6. In contrast, all bsFabs from bin F were inactive, while bsFabs from bins D and E remained antagonistic (data not shown). Altogether, the results revealed a strong association between CD80 competition bin and antagonist properties in the MLR assay.

T-cell signaling is determined largely by costimulatory and coinhibitory receptors, which control the function of TCR (Chen et al. Nat Rev Immunol (2013) 13(4):227-42.). These receptors are diverse, and their functions are influenced largely by context, as has been appreciated (Chen, supra). Costimulatory receptors such as CD27, OX40 (CD134), 4-1BB (CD137), or GITR (glucocorticoid-induced Tumor necrosis factor receptors related protein or CD357) with their respective activating ligands have become a major research focus within the Biologics community with efforts directed at their activation or inhibition for cancer and inflammatory immunotherapies, respectively (Edner et al. Nat Rev Drug Discov (2020) 19(12):860-83; Blanco et al. Clin Cancer Res (2021) 27(20):5457-64; Kraehenbuehl et al. Nat Rev Clin Oncol (2022) 19(1):37-50).

1. An antibody or antigen-binding fragment thereof that specifically binds to CD28, comprising an immunoglobulin single variable domain (IVSD) comprising a CDR-H1 sequence, a CDR-H2 sequence, and a CDR-H3 sequence, wherein:
a) the CDR-H1 sequence comprises an amino acid sequence set forth in SEQ ID NO: 1, the CDR-H2 sequence comprises an amino acid sequence set forth in SEQ ID NO: 2, and the CDR-H3 sequence comprises an amino acid sequence set forth in SEQ ID NO: 3;
b) the CDR-H1 sequence comprises an amino acid sequence set forth in SEQ ID NO: 4, the CDR-H2 sequence comprises an amino acid sequence set forth in SEQ ID NO: 5, and the CDR-H3 sequence comprises an amino acid sequence set forth in SEQ ID NO: 6;
c) the CDR-H1 sequence comprises an amino acid sequence set forth in SEQ ID NO: 7, the CDR-H2 sequence comprises an amino acid sequence set forth in SEQ ID NO: 8, and the CDR-H3 sequence comprises an amino acid sequence set forth in SEQ ID NO: 9;
d) the CDR-H1 sequence comprises an amino acid sequence set forth in SEQ ID NO: 10, the CDR-H2 sequence comprises an amino acid sequence set forth in SEQ ID NO: 11, and the CDR-H3 sequence comprises an amino acid sequence set forth in SEQ ID NO: 12;
e) the CDR-H1 sequence comprises an amino acid sequence set forth in SEQ ID NO: 13, the CDR-H2 sequence comprises an amino acid sequence set forth in SEQ ID NO: 14, and the CDR-H3 sequence comprises an amino acid sequence set forth in SEQ ID NO: 15;
f) the CDR-H1 sequence comprises an amino acid sequence set forth in SEQ ID NO: 16, the CDR-H2 sequence comprises an amino acid sequence set forth in SEQ ID NO: 17, and the CDR-H3 sequence comprises an amino acid sequence set forth in SEQ ID NO: 18;
g) the CDR-H1 sequence comprises an amino acid sequence set forth in SEQ ID NO: 19, the CDR-H2 sequence comprises an amino acid sequence set forth in SEQ ID NO: 20, and the CDR-H3 sequence comprises an amino acid sequence set forth in SEQ ID NO: 21;
h) the CDR-H1 sequence comprises an amino acid sequence set forth in SEQ ID NO: 22, the CDR-H2 sequence comprises an amino acid sequence set forth in SEQ ID NO: 23, and the CDR-H3 sequence comprises an amino acid sequence set forth in SEQ ID NO: 24;
i) the CDR-H1 sequence comprises an amino acid sequence set forth in SEQ ID NO: 25, the CDR-H2 sequence comprises an amino acid sequence set forth in SEQ ID NO: 26, and the CDR-H3 sequence comprises an amino acid sequence set forth in SEQ ID NO: 27;
j) the CDR-H1 sequence comprises an amino acid sequence set forth in SEQ ID NO: 28, the CDR-H2 sequence comprises an amino acid sequence set forth in SEQ ID NO: 29, and the CDR-H3 sequence comprises an amino acid sequence set forth in SEQ ID NO: 30;
k) the CDR-H1 sequence comprises an amino acid sequence set forth in SEQ ID NO: 31, the CDR-H2 sequence comprises an amino acid sequence set forth in SEQ ID NO: 32, and the CDR-H3 sequence comprises an amino acid sequence set forth in SEQ ID NO: 33;
l) the CDR-H1 sequence comprises an amino acid sequence set forth in SEQ ID NO: 34, the CDR-H2 sequence comprises an amino acid sequence set forth in SEQ ID NO: 35, and the CDR-H3 sequence comprises an amino acid sequence set forth in SEQ ID NO: 36;
m) the CDR-H1 sequence comprises an amino acid sequence set forth in SEQ ID NO: 37, the CDR-H2 sequence comprises an amino acid sequence set forth in SEQ ID NO: 38, and the CDR-H3 sequence comprises an amino acid sequence set forth in SEQ ID NO: 39;
n) the CDR-H1 sequence comprises an amino acid sequence set forth in SEQ ID NO: 40, the CDR-H2 sequence comprises an amino acid sequence set forth in SEQ ID NO: 41, and the CDR-H3 sequence comprises an amino acid sequence set forth in SEQ ID NO: 42;
o) the CDR-H1 sequence comprises an amino acid sequence set forth in SEQ ID NO: 43, the CDR-H2 sequence comprises an amino acid sequence set forth in SEQ ID NO: 44, and the CDR-H3 sequence comprises an amino acid sequence set forth in SEQ ID NO: 45;
p) the CDR-H1 sequence comprises an amino acid sequence set forth in SEQ ID NO: 46, the CDR-H2 sequence comprises an amino acid sequence set forth in SEQ ID NO: 47, and the CDR-H3 sequence comprises an amino acid sequence set forth in SEQ ID NO: 48;
q) the CDR-H1 sequence comprises an amino acid sequence set forth in SEQ ID NO: 49, the CDR-H2 sequence comprises an amino acid sequence set forth in SEQ ID NO: 50, and the CDR-H3 sequence comprises an amino acid sequence set forth in SEQ ID NO: 51;
r) the CDR-H1 sequence comprises an amino acid sequence set forth in SEQ ID NO: 52, the CDR-H2 sequence comprises an amino acid sequence set forth in SEQ ID NO: 53, and the CDR-H3 sequence comprises an amino acid sequence set forth in SEQ ID NO: 54;
s) the CDR-H1 sequence comprises an amino acid sequence set forth in SEQ ID NO: 55, the CDR-H2 sequence comprises an amino acid sequence set forth in SEQ ID NO: 56, and the CDR-H3 sequence comprises an amino acid sequence set forth in SEQ ID NO: 57;
t) the CDR-H1 sequence comprises an amino acid sequence set forth in SEQ ID NO: 58, the CDR-H2 sequence comprises an amino acid sequence set forth in SEQ ID NO: 59, and the CDR-H3 sequence comprises an amino acid sequence set forth in SEQ ID NO: 60;
u) the CDR-H1 sequence comprises an amino acid sequence set forth in SEQ ID NO: 61, the CDR-H2 sequence comprises an amino acid sequence set forth in SEQ ID NO: 62, and the CDR-H3 sequence comprises an amino acid sequence set forth in SEQ ID NO: 63;
v) the CDR-H1 sequence comprises an amino acid sequence set forth in SEQ ID NO: 64, the CDR-H2 sequence comprises an amino acid sequence set forth in SEQ ID NO: 65, and the CDR-H3 sequence comprises an amino acid sequence set forth in SEQ ID NO: 66;
w) the CDR-H1 sequence comprises an amino acid sequence set forth in SEQ ID NO: 67, the CDR-H2 sequence comprises an amino acid sequence set forth in SEQ ID NO: 68, and the CDR-H3 sequence comprises an amino acid sequence set forth in SEQ ID NO: 69;
x) the CDR-H1 sequence comprises an amino acid sequence set forth in SEQ ID NO: 70, the CDR-H2 sequence comprises an amino acid sequence set forth in SEQ ID NO: 71, and the CDR-H3 sequence comprises an amino acid sequence set forth in SEQ ID NO: 72;
y) the CDR-H1 sequence comprises an amino acid sequence set forth in SEQ ID NO: 73, the CDR-H2 sequence comprises an amino acid sequence set forth in SEQ ID NO: 74, and the CDR-H3 sequence comprises an amino acid sequence set forth in SEQ ID NO: 75;
z) the CDR-H1 sequence comprises an amino acid sequence set forth in SEQ ID NO: 76, the CDR-H2 sequence comprises an amino acid sequence set forth in SEQ ID NO: 77, and the CDR-H3 sequence comprises an amino acid sequence set forth in SEQ ID NO: 78;
aa) the CDR-H1 sequence comprises an amino acid sequence set forth in SEQ ID NO: 79, the CDR-H2 sequence comprises an amino acid sequence set forth in SEQ ID NO: 80, and the CDR-H3 sequence comprises an amino acid sequence set forth in SEQ ID NO: 81;
ab) the CDR-H1 sequence comprises an amino acid sequence set forth in SEQ ID NO: 82, the CDR-H2 sequence comprises an amino acid sequence set forth in SEQ ID NO: 83, and the CDR-H3 sequence comprises an amino acid sequence set forth in SEQ ID NO: 84;
ac) the CDR-H1 sequence comprises an amino acid sequence set forth in SEQ ID NO: 85, the CDR-H2 sequence comprises an amino acid sequence set forth in SEQ ID NO: 86, and the CDR-H3 sequence comprises an amino acid sequence set forth in SEQ ID NO: 87;
ad) the CDR-H1 sequence comprises an amino acid sequence set forth in SEQ ID NO: 88, the CDR-H2 sequence comprises an amino acid sequence set forth in SEQ ID NO: 89, and the CDR-H3 sequence comprises an amino acid sequence set forth in SEQ ID NO: 90;
ae) the CDR-H1 sequence comprises an amino acid sequence set forth in SEQ ID NO: 91, the CDR-H2 sequence comprises an amino acid sequence set forth in SEQ ID NO: 92, and the CDR-H3 sequence comprises an amino acid sequence set forth in SEQ ID NO: 93;
af) the CDR-H1 sequence comprises an amino acid sequence set forth in SEQ ID NO: 94, the CDR-H2 sequence comprises an amino acid sequence set forth in SEQ ID NO: 95, and the CDR-H3 sequence comprises an amino acid sequence set forth in SEQ ID NO: 96;
ag) the CDR-H1 sequence comprises an amino acid sequence set forth in SEQ ID NO: 97, the CDR-H2 sequence comprises an amino acid sequence set forth in SEQ ID NO: 98, and the CDR-H3 sequence comprises an amino acid sequence set forth in SEQ ID NO: 99;
ah) the CDR-H1 sequence comprises an amino acid sequence set forth in SEQ ID NO: 100, the CDR-H2 sequence comprises an amino acid sequence set forth in SEQ ID NO: 101, and the CDR-H3 sequence comprises an amino acid sequence set forth in SEQ ID NO: 102;
ai) the CDR-H1 sequence comprises an amino acid sequence set forth in SEQ ID NO: 103, the CDR-H2 sequence comprises an amino acid sequence set forth in SEQ ID NO: 104, and the CDR-H3 sequence comprises an amino acid sequence set forth in SEQ ID NO: 105;
aj) the CDR-H1 sequence comprises an amino acid sequence set forth in SEQ ID NO: 106, the CDR-H2 sequence comprises an amino acid sequence set forth in SEQ ID NO: 107, and the CDR-H3 sequence comprises an amino acid sequence set forth in SEQ ID NO: 108;
ak) the CDR-H1 sequence comprises an amino acid sequence set forth in SEQ ID NO: 109, the CDR-H2 sequence comprises an amino acid sequence set forth in SEQ ID NO: 110, and the CDR-H3 sequence comprises an amino acid sequence set forth in SEQ ID NO: 111;
al) the CDR-H1 sequence comprises an amino acid sequence set forth in SEQ ID NO: 112, the CDR-H2 sequence comprises an amino acid sequence set forth in SEQ ID NO: 113, and the CDR-H3 sequence comprises an amino acid sequence set forth in SEQ ID NO: 114;
am) the CDR-H1 sequence comprises an amino acid sequence set forth in SEQ ID NO: 115, the CDR-H2 sequence comprises an amino acid sequence set forth in SEQ ID NO: 116, and the CDR-H3 sequence comprises an amino acid sequence set forth in SEQ ID NO: 117;
an) the CDR-H1 sequence comprises an amino acid sequence set forth in SEQ ID NO: 118, the CDR-H2 sequence comprises an amino acid sequence set forth in SEQ ID NO: 119, and the CDR-H3 sequence comprises an amino acid sequence set forth in SEQ ID NO: 120;
ao) the CDR-H1 sequence comprises an amino acid sequence set forth in SEQ ID NO: 121, the CDR-H2 sequence comprises an amino acid sequence set forth in SEQ ID NO: 122, and the CDR-H3 sequence comprises an amino acid sequence set forth in SEQ ID NO: 123;
ap) the CDR-H1 sequence comprises an amino acid sequence set forth in SEQ ID NO: 124, the CDR-H2 sequence comprises an amino acid sequence set forth in SEQ ID NO: 125, and the CDR-H3 sequence comprises an amino acid sequence set forth in SEQ ID NO: 126;
aq) the CDR-H1 sequence comprises an amino acid sequence set forth in SEQ ID NO: 127, the CDR-H2 sequence comprises an amino acid sequence set forth in SEQ ID NO: 128, and the CDR-H3 sequence comprises an amino acid sequence set forth in SEQ ID NO: 129;
ar) the CDR-H1 sequence comprises an amino acid sequence set forth in SEQ ID NO: 130, the CDR-H2 sequence comprises an amino acid sequence set forth in SEQ ID NO: 131, and the CDR-H3 sequence comprises an amino acid sequence set forth in SEQ ID NO: 132;
as) the CDR-H1 sequence comprises an amino acid sequence set forth in SEQ ID NO: 133, the CDR-H2 sequence comprises an amino acid sequence set forth in SEQ ID NO: 134, and the CDR-H3 sequence comprises an amino acid sequence set forth in SEQ ID NO: 135;
at) the CDR-H1 sequence comprises an amino acid sequence set forth in SEQ ID NO: 136, the CDR-H2 sequence comprises an amino acid sequence set forth in SEQ ID NO: 137, and the CDR-H3 sequence comprises an amino acid sequence set forth in SEQ ID NO: 138;
au) the CDR-H1 sequence comprises an amino acid sequence set forth in SEQ ID NO: 139, the CDR-H2 sequence comprises an amino acid sequence set forth in SEQ ID NO: 140, and the CDR-H3 sequence comprises an amino acid sequence set forth in SEQ ID NO: 141;
av) the CDR-H1 sequence comprises an amino acid sequence set forth in SEQ ID NO: 142, the CDR-H2 sequence comprises an amino acid sequence set forth in SEQ ID NO: 143, and the CDR-H3 sequence comprises an amino acid sequence set forth in SEQ ID NO: 144;
aw) the CDR-H1 sequence comprises an amino acid sequence set forth in SEQ ID NO: 145, the CDR-H2 sequence comprises an amino acid sequence set forth in SEQ ID NO: 146, and the CDR-H3 sequence comprises an amino acid sequence set forth in SEQ ID NO: 147;
ax) the CDR-H1 sequence comprises an amino acid sequence set forth in SEQ ID NO: 148, the CDR-H2 sequence comprises an amino acid sequence set forth in SEQ ID NO: 149, and the CDR-H3 sequence comprises an amino acid sequence set forth in SEQ ID NO: 150;
ay) the CDR-H1 sequence comprises an amino acid sequence set forth in SEQ ID NO: 151, the CDR-H2 sequence comprises an amino acid sequence set forth in SEQ ID NO: 152, and the CDR-H3 sequence comprises an amino acid sequence set forth in SEQ ID NO: 153;
az) the CDR-H1 sequence comprises an amino acid sequence set forth in SEQ ID NO: 154, the CDR-H2 sequence comprises an amino acid sequence set forth in SEQ ID NO: 155, and the CDR-H3 sequence comprises an amino acid sequence set forth in SEQ ID NO: 156;
ba) the CDR-H1 sequence comprises an amino acid sequence set forth in SEQ ID NO: 157, the CDR-H2 sequence comprises an amino acid sequence set forth in SEQ ID NO: 158, and the CDR-H3 sequence comprises an amino acid sequence set forth in SEQ ID NO: 159;
bb) the CDR-H1 sequence comprises an amino acid sequence set forth in SEQ ID NO: 160, the CDR-H2 sequence comprises an amino acid sequence set forth in SEQ ID NO: 161, and the CDR-H3 sequence comprises an amino acid sequence set forth in SEQ ID NO: 162;
bc) the CDR-H1 sequence comprises an amino acid sequence set forth in SEQ ID NO: 163, the CDR-H2 sequence comprises an amino acid sequence set forth in SEQ ID NO: 164, and the CDR-H3 sequence comprises an amino acid sequence set forth in SEQ ID NO: 165;
bd) the CDR-H1 sequence comprises an amino acid sequence set forth in SEQ ID NO: 166, the CDR-H2 sequence comprises an amino acid sequence set forth in SEQ ID NO: 167, and the CDR-H3 sequence comprises an amino acid sequence set forth in SEQ ID NO: 168;
be) the CDR-H1 sequence comprises an amino acid sequence set forth in SEQ ID NO: 169, the CDR-H2 sequence comprises an amino acid sequence set forth in SEQ ID NO: 170, and the CDR-H3 sequence comprises an amino acid sequence set forth in SEQ ID NO: 171;
bf) the CDR-H1 sequence comprises an amino acid sequence set forth in SEQ ID NO: 172, the CDR-H2 sequence comprises an amino acid sequence set forth in SEQ ID NO: 173, and the CDR-H3 sequence comprises an amino acid sequence set forth in SEQ ID NO: 174;
bg) the CDR-H1 sequence comprises an amino acid sequence set forth in SEQ ID NO: 175, the CDR-H2 sequence comprises an amino acid sequence set forth in SEQ ID NO: 176, and the CDR-H3 sequence comprises an amino acid sequence set forth in SEQ ID NO: 177;
bh) the CDR-H1 sequence comprises an amino acid sequence set forth in SEQ ID NO: 178, the CDR-H2 sequence comprises an amino acid sequence set forth in SEQ ID NO: 179, and the CDR-H3 sequence comprises an amino acid sequence set forth in SEQ ID NO: 180;
bi) the CDR-H1 sequence comprises an amino acid sequence set forth in SEQ ID NO: 181, the CDR-H2 sequence comprises an amino acid sequence set forth in SEQ ID NO: 182, and the CDR-H3 sequence comprises an amino acid sequence set forth in SEQ ID NO: 183;
bj) the CDR-H1 sequence comprises an amino acid sequence set forth in SEQ ID NO: 184, the CDR-H2 sequence comprises an amino acid sequence set forth in SEQ ID NO: 185, and the CDR-H3 sequence comprises an amino acid sequence set forth in SEQ ID NO: 186;
bk) the CDR-H1 sequence comprises an amino acid sequence set forth in SEQ ID NO: 187, the CDR-H2 sequence comprises an amino acid sequence set forth in SEQ ID NO: 188, and the CDR-H3 sequence comprises an amino acid sequence set forth in SEQ ID NO: 189;
bl) the CDR-H1 sequence comprises an amino acid sequence set forth in SEQ ID NO: 190, the CDR-H2 sequence comprises an amino acid sequence set forth in SEQ ID NO: 191, and the CDR-H3 sequence comprises an amino acid sequence set forth in SEQ ID NO: 192;
bm) the CDR-H1 sequence comprises an amino acid sequence set forth in SEQ ID NO: 193, the CDR-H2 sequence comprises an amino acid sequence set forth in SEQ ID NO: 194, and the CDR-H3 sequence comprises an amino acid sequence set forth in SEQ ID NO: 195;
bn) the CDR-H1 sequence comprises an amino acid sequence set forth in SEQ ID NO: 196, the CDR-H2 sequence comprises an amino acid sequence set forth in SEQ ID NO: 197, and the CDR-H3 sequence comprises an amino acid sequence set forth in SEQ ID NO: 198;
bo) the CDR-H1 sequence comprises an amino acid sequence set forth in SEQ ID NO: 199, the CDR-H2 sequence comprises an amino acid sequence set forth in SEQ ID NO: 200, and the CDR-H3 sequence comprises an amino acid sequence set forth in SEQ ID NO: 201;
bp) the CDR-H1 sequence comprises an amino acid sequence set forth in SEQ ID NO: 202, the CDR-H2 sequence comprises an amino acid sequence set forth in SEQ ID NO: 203, and the CDR-H3 sequence comprises an amino acid sequence set forth in SEQ ID NO: 204;
bq) the CDR-H1 sequence comprises an amino acid sequence set forth in SEQ ID NO: 205, the CDR-H2 sequence comprises an amino acid sequence set forth in SEQ ID NO: 206, and the CDR-H3 sequence comprises an amino acid sequence set forth in SEQ ID NO: 207;
br) the CDR-H1 sequence comprises an amino acid sequence set forth in SEQ ID NO: 208, the CDR-H2 sequence comprises an amino acid sequence set forth in SEQ ID NO: 209, and the CDR-H3 sequence comprises an amino acid sequence set forth in SEQ ID NO: 210;
bs) the CDR-H1 sequence comprises an amino acid sequence set forth in SEQ ID NO: 211, the CDR-H2 sequence comprises an amino acid sequence set forth in SEQ ID NO: 212, and the CDR-H3 sequence comprises an amino acid sequence set forth in SEQ ID NO: 213;
bt) the CDR-H1 sequence comprises an amino acid sequence set forth in SEQ ID NO: 214, the CDR-H2 sequence comprises an amino acid sequence set forth in SEQ ID NO: 215, and the CDR-H3 sequence comprises an amino acid sequence set forth in SEQ ID NO: 216;
bu) the CDR-H1 sequence comprises an amino acid sequence set forth in SEQ ID NO: 217, the CDR-H2 sequence comprises an amino acid sequence set forth in SEQ ID NO: 218, and the CDR-H3 sequence comprises an amino acid sequence set forth in SEQ ID NO: 219;
by) the CDR-H1 sequence comprises an amino acid sequence set forth in SEQ ID NO: 220, the CDR-H2 sequence comprises an amino acid sequence set forth in SEQ ID NO: 221, and the CDR-H3 sequence comprises an amino acid sequence set forth in SEQ ID NO: 222;
bw) the CDR-H1 sequence comprises an amino acid sequence set forth in SEQ ID NO: 223, the CDR-H2 sequence comprises an amino acid sequence set forth in SEQ ID NO: 224, and the CDR-H3 sequence comprises an amino acid sequence set forth in SEQ ID NO: 225;
bx) the CDR-H1 sequence comprises an amino acid sequence set forth in SEQ ID NO: 226, the CDR-H2 sequence comprises an amino acid sequence set forth in SEQ ID NO: 227, and the CDR-H3 sequence comprises an amino acid sequence set forth in SEQ ID NO: 228;
by) the CDR-H1 sequence comprises an amino acid sequence set forth in SEQ ID NO: 229, the CDR-H2 sequence comprises an amino acid sequence set forth in SEQ ID NO: 230, and the CDR-H3 sequence comprises an amino acid sequence set forth in SEQ ID NO: 231;
bz) the CDR-H1 sequence comprises an amino acid sequence set forth in SEQ ID NO: 232, the CDR-H2 sequence comprises an amino acid sequence set forth in SEQ ID NO: 233, and the CDR-H3 sequence comprises an amino acid sequence set forth in SEQ ID NO: 234;
ca) the CDR-H1 sequence comprises an amino acid sequence set forth in SEQ ID NO: 235, the CDR-H2 sequence comprises an amino acid sequence set forth in SEQ ID NO: 236, and the CDR-H3 sequence comprises an amino acid sequence set forth in SEQ ID NO: 237;
cb) the CDR-H1 sequence comprises an amino acid sequence set forth in SEQ ID NO: 238, the CDR-H2 sequence comprises an amino acid sequence set forth in SEQ ID NO: 239, and the CDR-H3 sequence comprises an amino acid sequence set forth in SEQ ID NO: 240;
cc) the CDR-H1 sequence comprises an amino acid sequence set forth in SEQ ID NO: 241, the CDR-H2 sequence comprises an amino acid sequence set forth in SEQ ID NO: 242, and the CDR-H3 sequence comprises an amino acid sequence set forth in SEQ ID NO: 243;
cd) the CDR-H1 sequence comprises an amino acid sequence set forth in SEQ ID NO: 244, the CDR-H2 sequence comprises an amino acid sequence set forth in SEQ ID NO: 245, and the CDR-H3 sequence comprises an amino acid sequence set forth in SEQ ID NO: 246;
ce) the CDR-H1 sequence comprises an amino acid sequence set forth in SEQ ID NO: 247, the CDR-H2 sequence comprises an amino acid sequence set forth in SEQ ID NO: 248, and the CDR-H3 sequence comprises an amino acid sequence set forth in SEQ ID NO: 249;
cf) the CDR-H1 sequence comprises an amino acid sequence set forth in SEQ ID NO: 250, the CDR-H2 sequence comprises an amino acid sequence set forth in SEQ ID NO: 251, and the CDR-H3 sequence comprises an amino acid sequence set forth in SEQ ID NO: 252;
cg) the CDR-H1 sequence comprises an amino acid sequence set forth in SEQ ID NO: 253, the CDR-H2 sequence comprises an amino acid sequence set forth in SEQ ID NO: 254, and the CDR-H3 sequence comprises an amino acid sequence set forth in SEQ ID NO: 255;
ch) the CDR-H1 sequence comprises an amino acid sequence set forth in SEQ ID NO: 256, the CDR-H2 sequence comprises an amino acid sequence set forth in SEQ ID NO: 257, and the CDR-H3 sequence comprises an amino acid sequence set forth in SEQ ID NO: 258;
ci) the CDR-H1 sequence comprises an amino acid sequence set forth in SEQ ID NO: 259, the CDR-H2 sequence comprises an amino acid sequence set forth in SEQ ID NO: 260, and the CDR-H3 sequence comprises an amino acid sequence set forth in SEQ ID NO: 261;
cj) the CDR-H1 sequence comprises an amino acid sequence set forth in SEQ ID NO: 262, the CDR-H2 sequence comprises an amino acid sequence set forth in SEQ ID NO: 263, and the CDR-H3 sequence comprises an amino acid sequence set forth in SEQ ID NO: 264;
ck) the CDR-H1 sequence comprises an amino acid sequence set forth in SEQ ID NO: 265, the CDR-H2 sequence comprises an amino acid sequence set forth in SEQ ID NO: 266, and the CDR-H3 sequence comprises an amino acid sequence set forth in SEQ ID NO: 267;
cl) the CDR-H1 sequence comprises an amino acid sequence set forth in SEQ ID NO: 268, the CDR-H2 sequence comprises an amino acid sequence set forth in SEQ ID NO: 269, and the CDR-H3 sequence comprises an amino acid sequence set forth in SEQ ID NO: 270;
cm) the CDR-H1 sequence comprises an amino acid sequence set forth in SEQ ID NO: 271, the CDR-H2 sequence comprises an amino acid sequence set forth in SEQ ID NO: 272, and the CDR-H3 sequence comprises an amino acid sequence set forth in SEQ ID NO: 273;
cn) the CDR-H1 sequence comprises an amino acid sequence set forth in SEQ ID NO: 274, the CDR-H2 sequence comprises an amino acid sequence set forth in SEQ ID NO: 275, and the CDR-H3 sequence comprises an amino acid sequence set forth in SEQ ID NO: 276;
co) the CDR-H1 sequence comprises an amino acid sequence set forth in SEQ ID NO: 277, the CDR-H2 sequence comprises an amino acid sequence set forth in SEQ ID NO: 278, and the CDR-H3 sequence comprises an amino acid sequence set forth in SEQ ID NO: 279;
cp) the CDR-H1 sequence comprises an amino acid sequence set forth in SEQ ID NO: 280, the CDR-H2 sequence comprises an amino acid sequence set forth in SEQ ID NO: 281, and the CDR-H3 sequence comprises an amino acid sequence set forth in SEQ ID NO: 282;
cq) the CDR-H1 sequence comprises an amino acid sequence set forth in SEQ ID NO: 283, the CDR-H2 sequence comprises an amino acid sequence set forth in SEQ ID NO: 284, and the CDR-H3 sequence comprises an amino acid sequence set forth in SEQ ID NO: 285;
cr) the CDR-H1 sequence comprises an amino acid sequence set forth in SEQ ID NO: 286, the CDR-H2 sequence comprises an amino acid sequence set forth in SEQ ID NO: 287, and the CDR-H3 sequence comprises an amino acid sequence set forth in SEQ ID NO: 288;
cs) the CDR-H1 sequence comprises an amino acid sequence set forth in SEQ ID NO: 289, the CDR-H2 sequence comprises an amino acid sequence set forth in SEQ ID NO: 290, and the CDR-H3 sequence comprises an amino acid sequence set forth in SEQ ID NO: 291;
ct) the CDR-H1 sequence comprises an amino acid sequence set forth in SEQ ID NO: 292, the CDR-H2 sequence comprises an amino acid sequence set forth in SEQ ID NO: 293, and the CDR-H3 sequence comprises an amino acid sequence set forth in SEQ ID NO: 294;
cu) the CDR-H1 sequence comprises an amino acid sequence set forth in SEQ ID NO: 295, the CDR-H2 sequence comprises an amino acid sequence set forth in SEQ ID NO: 296, and the CDR-H3 sequence comprises an amino acid sequence set forth in SEQ ID NO: 297;
cv) the CDR-H1 sequence comprises an amino acid sequence set forth in SEQ ID NO: 298, the CDR-H2 sequence comprises an amino acid sequence set forth in SEQ ID NO: 299, and the CDR-H3 sequence comprises an amino acid sequence set forth in SEQ ID NO: 300;
cw) the CDR-H1 sequence comprises an amino acid sequence set forth in SEQ ID NO: 301, the CDR-H2 sequence comprises an amino acid sequence set forth in SEQ ID NO: 302, and the CDR-H3 sequence comprises an amino acid sequence set forth in SEQ ID NO: 303;
cx) the CDR-H1 sequence comprises an amino acid sequence set forth in SEQ ID NO: 304, the CDR-H2 sequence comprises an amino acid sequence set forth in SEQ ID NO: 305, and the CDR-H3 sequence comprises an amino acid sequence set forth in SEQ ID NO: 306;
cy) the CDR-H1 sequence comprises an amino acid sequence set forth in SEQ ID NO: 307, the CDR-H2 sequence comprises an amino acid sequence set forth in SEQ ID NO: 308, and the CDR-H3 sequence comprises an amino acid sequence set forth in SEQ ID NO: 309;
cz) the CDR-H1 sequence comprises an amino acid sequence set forth in SEQ ID NO: 310, the CDR-H2 sequence comprises an amino acid sequence set forth in SEQ ID NO: 311, and the CDR-H3 sequence comprises an amino acid sequence set forth in SEQ ID NO: 312;
da) the CDR-H1 sequence comprises an amino acid sequence set forth in SEQ ID NO: 313, the CDR-H2 sequence comprises an amino acid sequence set forth in SEQ ID NO: 314, and the CDR-H3 sequence comprises an amino acid sequence set forth in SEQ ID NO: 315;
db) the CDR-H1 sequence comprises an amino acid sequence set forth in SEQ ID NO: 316, the CDR-H2 sequence comprises an amino acid sequence set forth in SEQ ID NO: 317, and the CDR-H3 sequence comprises an amino acid sequence set forth in SEQ ID NO: 318;
dc) the CDR-H1 sequence comprises an amino acid sequence set forth in SEQ ID NO: 319, the CDR-H2 sequence comprises an amino acid sequence set forth in SEQ ID NO: 320, and the CDR-H3 sequence comprises an amino acid sequence set forth in SEQ ID NO: 321;
dd) the CDR-H1 sequence comprises an amino acid sequence set forth in SEQ ID NO: 322, the CDR-H2 sequence comprises an amino acid sequence set forth in SEQ ID NO: 323, and the CDR-H3 sequence comprises an amino acid sequence set forth in SEQ ID NO: 324;
de) the CDR-H1 sequence comprises an amino acid sequence set forth in SEQ ID NO: 325, the CDR-H2 sequence comprises an amino acid sequence set forth in SEQ ID NO: 326, and the CDR-H3 sequence comprises an amino acid sequence set forth in SEQ ID NO: 327;
df) the CDR-H1 sequence comprises an amino acid sequence set forth in SEQ ID NO: 328, the CDR-H2 sequence comprises an amino acid sequence set forth in SEQ ID NO: 329, and the CDR-H3 sequence comprises an amino acid sequence set forth in SEQ ID NO: 330;
dg) the CDR-H1 sequence comprises an amino acid sequence set forth in SEQ ID NO: 331, the CDR-H2 sequence comprises an amino acid sequence set forth in SEQ ID NO: 332, and the CDR-H3 sequence comprises an amino acid sequence set forth in SEQ ID NO: 333;
dh) the CDR-H1 sequence comprises an amino acid sequence set forth in SEQ ID NO: 334, the CDR-H2 sequence comprises an amino acid sequence set forth in SEQ ID NO: 335, and the CDR-H3 sequence comprises an amino acid sequence set forth in SEQ ID NO: 336;
di) the CDR-H1 sequence comprises an amino acid sequence set forth in SEQ ID NO: 337, the CDR-H2 sequence comprises an amino acid sequence set forth in SEQ ID NO: 338, and the CDR-H3 sequence comprises an amino acid sequence set forth in SEQ ID NO: 339;
dj) the CDR-H1 sequence comprises an amino acid sequence set forth in SEQ ID NO: 340, the CDR-H2 sequence comprises an amino acid sequence set forth in SEQ ID NO: 341, and the CDR-H3 sequence comprises an amino acid sequence set forth in SEQ ID NO: 342;
dk) the CDR-H1 sequence comprises an amino acid sequence set forth in SEQ ID NO: 343, the CDR-H2 sequence comprises an amino acid sequence set forth in SEQ ID NO: 344, and the CDR-H3 sequence comprises an amino acid sequence set forth in SEQ ID NO: 345;
dl) the CDR-H1 sequence comprises an amino acid sequence set forth in SEQ ID NO: 346, the CDR-H2 sequence comprises an amino acid sequence set forth in SEQ ID NO: 347, and the CDR-H3 sequence comprises an amino acid sequence set forth in SEQ ID NO: 348;
dm) the CDR-H1 sequence comprises an amino acid sequence set forth in SEQ ID NO: 349, the CDR-H2 sequence comprises an amino acid sequence set forth in SEQ ID NO: 350, and the CDR-H3 sequence comprises an amino acid sequence set forth in SEQ ID NO: 351;
dn) the CDR-H1 sequence comprises an amino acid sequence set forth in SEQ ID NO: 352, the CDR-H2 sequence comprises an amino acid sequence set forth in SEQ ID NO: 353, and the CDR-H3 sequence comprises an amino acid sequence set forth in SEQ ID NO: 354;
do) the CDR-H1 sequence comprises an amino acid sequence set forth in SEQ ID NO: 355, the CDR-H2 sequence comprises an amino acid sequence set forth in SEQ ID NO: 356, and the CDR-H3 sequence comprises an amino acid sequence set forth in SEQ ID NO: 357;
dp) the CDR-H1 sequence comprises an amino acid sequence set forth in SEQ ID NO: 358, the CDR-H2 sequence comprises an amino acid sequence set forth in SEQ ID NO: 359, and the CDR-H3 sequence comprises an amino acid sequence set forth in SEQ ID NO: 360;
dq) the CDR-H1 sequence comprises an amino acid sequence set forth in SEQ ID NO: 361, the CDR-H2 sequence comprises an amino acid sequence set forth in SEQ ID NO: 362, and the CDR-H3 sequence comprises an amino acid sequence set forth in SEQ ID NO: 363;
dr) the CDR-H1 sequence comprises an amino acid sequence set forth in SEQ ID NO: 364, the CDR-H2 sequence comprises an amino acid sequence set forth in SEQ ID NO: 365, and the CDR-H3 sequence comprises an amino acid sequence set forth in SEQ ID NO: 366;
ds) the CDR-H1 sequence comprises an amino acid sequence set forth in SEQ ID NO: 367, the CDR-H2 sequence comprises an amino acid sequence set forth in SEQ ID NO: 368, and the CDR-H3 sequence comprises an amino acid sequence set forth in SEQ ID NO: 369;
dt) the CDR-H1 sequence comprises an amino acid sequence set forth in SEQ ID NO: 370, the CDR-H2 sequence comprises an amino acid sequence set forth in SEQ ID NO: 371, and the CDR-H3 sequence comprises an amino acid sequence set forth in SEQ ID NO: 372;
du) the CDR-H1 sequence comprises an amino acid sequence set forth in SEQ ID NO: 373, the CDR-H2 sequence comprises an amino acid sequence set forth in SEQ ID NO: 374, and the CDR-H3 sequence comprises an amino acid sequence set forth in SEQ ID NO: 375;
dv) the CDR-H1 sequence comprises an amino acid sequence set forth in SEQ ID NO: 376, the CDR-H2 sequence comprises an amino acid sequence set forth in SEQ ID NO: 377, and the CDR-H3 sequence comprises an amino acid sequence set forth in SEQ ID NO: 378;
dw) the CDR-H1 sequence comprises an amino acid sequence set forth in SEQ ID NO: 379, the CDR-H2 sequence comprises an amino acid sequence set forth in SEQ ID NO: 380, and the CDR-H3 sequence comprises an amino acid sequence set forth in SEQ ID NO: 381;
dx) the CDR-H1 sequence comprises an amino acid sequence set forth in SEQ ID NO: 382, the CDR-H2 sequence comprises an amino acid sequence set forth in SEQ ID NO: 383, and the CDR-H3 sequence comprises an amino acid sequence set forth in SEQ ID NO: 384;
dy) the CDR-H1 sequence comprises an amino acid sequence set forth in SEQ ID NO: 385, the CDR-H2 sequence comprises an amino acid sequence set forth in SEQ ID NO: 386, and the CDR-H3 sequence comprises an amino acid sequence set forth in SEQ ID NO: 387;
dz) the CDR-H1 sequence comprises an amino acid sequence set forth in SEQ ID NO: 388, the CDR-H2 sequence comprises an amino acid sequence set forth in SEQ ID NO: 389, and the CDR-H3 sequence comprises an amino acid sequence set forth in SEQ ID NO: 390;
ea) the CDR-H1 sequence comprises an amino acid sequence set forth in SEQ ID NO: 391, the CDR-H2 sequence comprises an amino acid sequence set forth in SEQ ID NO: 392, and the CDR-H3 sequence comprises an amino acid sequence set forth in SEQ ID NO: 393;
eb) the CDR-H1 sequence comprises an amino acid sequence set forth in SEQ ID NO: 394, the CDR-H2 sequence comprises an amino acid sequence set forth in SEQ ID NO: 395, and the CDR-H3 sequence comprises an amino acid sequence set forth in SEQ ID NO: 396;
ec) the CDR-H1 sequence comprises an amino acid sequence set forth in SEQ ID NO: 397, the CDR-H2 sequence comprises an amino acid sequence set forth in SEQ ID NO: 398, and the CDR-H3 sequence comprises an amino acid sequence set forth in SEQ ID NO: 399;
ed) the CDR-H1 sequence comprises an amino acid sequence set forth in SEQ ID NO: 400, the CDR-H2 sequence comprises an amino acid sequence set forth in SEQ ID NO: 401, and the CDR-H3 sequence comprises an amino acid sequence set forth in SEQ ID NO: 402;
ee) the CDR-H1 sequence comprises an amino acid sequence set forth in SEQ ID NO: 403, the CDR-H2 sequence comprises an amino acid sequence set forth in SEQ ID NO: 404, and the CDR-H3 sequence comprises an amino acid sequence set forth in SEQ ID NO: 405;
ef) the CDR-H1 sequence comprises an amino acid sequence set forth in SEQ ID NO: 406, the CDR-H2 sequence comprises an amino acid sequence set forth in SEQ ID NO: 407, and the CDR-H3 sequence comprises an amino acid sequence set forth in SEQ ID NO: 408;
eg) the CDR-H1 sequence comprises an amino acid sequence set forth in SEQ ID NO: 409, the CDR-H2 sequence comprises an amino acid sequence set forth in SEQ ID NO: 410, and the CDR-H3 sequence comprises an amino acid sequence set forth in SEQ ID NO: 411;
eh) the CDR-H1 sequence comprises an amino acid sequence set forth in SEQ ID NO: 412, the CDR-H2 sequence comprises an amino acid sequence set forth in SEQ ID NO: 413, and the CDR-H3 sequence comprises an amino acid sequence set forth in SEQ ID NO: 414;
ei) the CDR-H1 sequence comprises an amino acid sequence set forth in SEQ ID NO: 415, the CDR-H2 sequence comprises an amino acid sequence set forth in SEQ ID NO: 416, and the CDR-H3 sequence comprises an amino acid sequence set forth in SEQ ID NO: 417;
ej) the CDR-H1 sequence comprises an amino acid sequence set forth in SEQ ID NO: 418, the CDR-H2 sequence comprises an amino acid sequence set forth in SEQ ID NO: 419, and the CDR-H3 sequence comprises an amino acid sequence set forth in SEQ ID NO: 420;
ek) the CDR-H1 sequence comprises an amino acid sequence set forth in SEQ ID NO: 421, the CDR-H2 sequence comprises an amino acid sequence set forth in SEQ ID NO: 422, and the CDR-H3 sequence comprises an amino acid sequence set forth in SEQ ID NO: 423;
el) the CDR-H1 sequence comprises an amino acid sequence set forth in SEQ ID NO: 424, the CDR-H2 sequence comprises an amino acid sequence set forth in SEQ ID NO: 425, and the CDR-H3 sequence comprises an amino acid sequence set forth in SEQ ID NO: 426;
em) the CDR-H1 sequence comprises an amino acid sequence set forth in SEQ ID NO: 427, the CDR-H2 sequence comprises an amino acid sequence set forth in SEQ ID NO: 428, and the CDR-H3 sequence comprises an amino acid sequence set forth in SEQ ID NO: 429;
en) the CDR-H1 sequence comprises an amino acid sequence set forth in SEQ ID NO: 430, the CDR-H2 sequence comprises an amino acid sequence set forth in SEQ ID NO: 431, and the CDR-H3 sequence comprises an amino acid sequence set forth in SEQ ID NO: 432;
eo) the CDR-H1 sequence comprises an amino acid sequence set forth in SEQ ID NO: 433, the CDR-H2 sequence comprises an amino acid sequence set forth in SEQ ID NO: 434, and the CDR-H3 sequence comprises an amino acid sequence set forth in SEQ ID NO: 435;
ep) the CDR-H1 sequence comprises an amino acid sequence set forth in SEQ ID NO: 436, the CDR-H2 sequence comprises an amino acid sequence set forth in SEQ ID NO: 437, and the CDR-H3 sequence comprises an amino acid sequence set forth in SEQ ID NO: 438;
eq) the CDR-H1 sequence comprises an amino acid sequence set forth in SEQ ID NO: 439, the CDR-H2 sequence comprises an amino acid sequence set forth in SEQ ID NO: 440, and the CDR-H3 sequence comprises an amino acid sequence set forth in SEQ ID NO: 441;
er) the CDR-H1 sequence comprises an amino acid sequence set forth in SEQ ID NO: 442, the CDR-H2 sequence comprises an amino acid sequence set forth in SEQ ID NO: 443, and the CDR-H3 sequence comprises an amino acid sequence set forth in SEQ ID NO: 444;
es) the CDR-H1 sequence comprises an amino acid sequence set forth in SEQ ID NO: 445, the CDR-H2 sequence comprises an amino acid sequence set forth in SEQ ID NO: 446, and the CDR-H3 sequence comprises an amino acid sequence set forth in SEQ ID NO: 447;
et) the CDR-H1 sequence comprises an amino acid sequence set forth in SEQ ID NO: 448, the CDR-H2 sequence comprises an amino acid sequence set forth in SEQ ID NO: 449, and the CDR-H3 sequence comprises an amino acid sequence set forth in SEQ ID NO: 450; or
eu) the CDR-H1 sequence comprises an amino acid sequence set forth in SEQ ID NO: 451, the CDR-H2 sequence comprises an amino acid sequence set forth in SEQ ID NO: 452, and the CDR-H3 sequence comprises an amino acid sequence set forth in SEQ ID NO: 453.

2. The antibody or antigen-binding fragment thereof of claim 1, wherein the ISVD comprises a VHH domain, wherein:
a) the VHH domain comprises an amino acid sequence set forth in SEQ ID NO: 454;
b) the VHH domain comprises an amino acid sequence set forth in SEQ ID NO: 455;
c) the VHH domain comprises an amino acid sequence set forth in SEQ ID NO: 456;
d) the VHH domain comprises an amino acid sequence set forth in SEQ ID NO: 457;
e) the VHH domain comprises an amino acid sequence set forth in SEQ ID NO: 458;
f) the VHH domain comprises an amino acid sequence set forth in SEQ ID NO: 459;
g) the VHH domain comprises an amino acid sequence set forth in SEQ ID NO: 460;
h) the VHH domain comprises an amino acid sequence set forth in SEQ ID NO: 461;
i) the VHH domain comprises an amino acid sequence set forth in SEQ ID NO: 462;
j) the VHH domain comprises an amino acid sequence set forth in SEQ ID NO: 463;
k) the VHH domain comprises an amino acid sequence set forth in SEQ ID NO: 464;
l) the VHH domain comprises an amino acid sequence set forth in SEQ ID NO: 465;
m) the VHH domain comprises an amino acid sequence set forth in SEQ ID NO: 466;
n) the VHH domain comprises an amino acid sequence set forth in SEQ ID NO: 467;
o) the VHH domain comprises an amino acid sequence set forth in SEQ ID NO: 468;
p) the VHH domain comprises an amino acid sequence set forth in SEQ ID NO: 469;
q) the VHH domain comprises an amino acid sequence set forth in SEQ ID NO: 470;
r) the VHH domain comprises an amino acid sequence set forth in SEQ ID NO: 471;
s) the VHH domain comprises an amino acid sequence set forth in SEQ ID NO: 472;
t) the VHH domain comprises an amino acid sequence set forth in SEQ ID NO: 473;
u) the VHH domain comprises an amino acid sequence set forth in SEQ ID NO: 474;
v) the VHH domain comprises an amino acid sequence set forth in SEQ ID NO: 475;
w) the VHH domain comprises an amino acid sequence set forth in SEQ ID NO: 476;
x) the VHH domain comprises an amino acid sequence set forth in SEQ ID NO: 477;
y) the VHH domain comprises an amino acid sequence set forth in SEQ ID NO: 478;
z) the VHH domain comprises an amino acid sequence set forth in SEQ ID NO: 479;
aa) the VHH domain comprises an amino acid sequence set forth in SEQ ID NO: 480;
ab) the VHH domain comprises an amino acid sequence set forth in SEQ ID NO: 481;
ac) the VHH domain comprises an amino acid sequence set forth in SEQ ID NO: 482;
ad) the VHH domain comprises an amino acid sequence set forth in SEQ ID NO: 483;
ae) the VHH domain comprises an amino acid sequence set forth in SEQ ID NO: 484;
af) the VHH domain comprises an amino acid sequence set forth in SEQ ID NO: 485;
ag) the VHH domain comprises an amino acid sequence set forth in SEQ ID NO: 486;
ah) the VHH domain comprises an amino acid sequence set forth in SEQ ID NO: 487;
ai) the VHH domain comprises an amino acid sequence set forth in SEQ ID NO: 488;
aj) the VHH domain comprises an amino acid sequence set forth in SEQ ID NO: 489;
ak) the VHH domain comprises an amino acid sequence set forth in SEQ ID NO: 490;
al) the VHH domain comprises an amino acid sequence set forth in SEQ ID NO: 491;
am) the VHH domain comprises an amino acid sequence set forth in SEQ ID NO: 492;
an) the VHH domain comprises an amino acid sequence set forth in SEQ ID NO: 493;
ao) the VHH domain comprises an amino acid sequence set forth in SEQ ID NO: 494;
ap) the VHH domain comprises an amino acid sequence set forth in SEQ ID NO: 495;
aq) the VHH domain comprises an amino acid sequence set forth in SEQ ID NO: 496;
ar) the VHH domain comprises an amino acid sequence set forth in SEQ ID NO: 497;
as) the VHH domain comprises an amino acid sequence set forth in SEQ ID NO: 498;
at) the VHH domain comprises an amino acid sequence set forth in SEQ ID NO: 499;
au) the VHH domain comprises an amino acid sequence set forth in SEQ ID NO: 500;
av) the VHH domain comprises an amino acid sequence set forth in SEQ ID NO: 501;
aw) the VHH domain comprises an amino acid sequence set forth in SEQ ID NO: 502;
ax) the VHH domain comprises an amino acid sequence set forth in SEQ ID NO: 503;
ay) the VHH domain comprises an amino acid sequence set forth in SEQ ID NO: 504;
az) the VHH domain comprises an amino acid sequence set forth in SEQ ID NO: 505;
ba) the VHH domain comprises an amino acid sequence set forth in SEQ ID NO: 506;
bb) the VHH domain comprises an amino acid sequence set forth in SEQ ID NO: 507;
bc) the VHH domain comprises an amino acid sequence set forth in SEQ ID NO: 508;
bd) the VHH domain comprises an amino acid sequence set forth in SEQ ID NO: 509;
be) the VHH domain comprises an amino acid sequence set forth in SEQ ID NO: 510;
bf) the VHH domain comprises an amino acid sequence set forth in SEQ ID NO: 511;
bg) the VHH domain comprises an amino acid sequence set forth in SEQ ID NO: 512;
bh) the VHH domain comprises an amino acid sequence set forth in SEQ ID NO: 513;
bi) the VHH domain comprises an amino acid sequence set forth in SEQ ID NO: 514;
bj) the VHH domain comprises an amino acid sequence set forth in SEQ ID NO: 515;
bk) the VHH domain comprises an amino acid sequence set forth in SEQ ID NO: 516;
bl) the VHH domain comprises an amino acid sequence set forth in SEQ ID NO: 517;
bm) the VHH domain comprises an amino acid sequence set forth in SEQ ID NO: 518;
bn) the VHH domain comprises an amino acid sequence set forth in SEQ ID NO: 519;
bo) the VHH domain comprises an amino acid sequence set forth in SEQ ID NO: 520;
bp) the VHH domain comprises an amino acid sequence set forth in SEQ ID NO: 521;
bq) the VHH domain comprises an amino acid sequence set forth in SEQ ID NO: 522;
br) the VHH domain comprises an amino acid sequence set forth in SEQ ID NO: 523;
bs) the VHH domain comprises an amino acid sequence set forth in SEQ ID NO: 524;
bt) the VHH domain comprises an amino acid sequence set forth in SEQ ID NO: 525;
bu) the VHH domain comprises an amino acid sequence set forth in SEQ ID NO: 526;
by) the VHH domain comprises an amino acid sequence set forth in SEQ ID NO: 527;
bw) the VHH domain comprises an amino acid sequence set forth in SEQ ID NO: 528;
bx) the VHH domain comprises an amino acid sequence set forth in SEQ ID NO: 529;
by) the VHH domain comprises an amino acid sequence set forth in SEQ ID NO: 530;
bz) the VHH domain comprises an amino acid sequence set forth in SEQ ID NO: 531;
ca) the VHH domain comprises an amino acid sequence set forth in SEQ ID NO: 532;
cb) the VHH domain comprises an amino acid sequence set forth in SEQ ID NO: 533;
cc) the VHH domain comprises an amino acid sequence set forth in SEQ ID NO: 534;
cd) the VHH domain comprises an amino acid sequence set forth in SEQ ID NO: 535;
ce) the VHH domain comprises an amino acid sequence set forth in SEQ ID NO: 536;
cf) the VHH domain comprises an amino acid sequence set forth in SEQ ID NO: 537;
cg) the VHH domain comprises an amino acid sequence set forth in SEQ ID NO: 538;
ch) the VHH domain comprises an amino acid sequence set forth in SEQ ID NO: 539;
ci) the VHH domain comprises an amino acid sequence set forth in SEQ ID NO: 540;
cj) the VHH domain comprises an amino acid sequence set forth in SEQ ID NO: 541;
ck) the VHH domain comprises an amino acid sequence set forth in SEQ ID NO: 542;
cl) the VHH domain comprises an amino acid sequence set forth in SEQ ID NO: 543;
cm) the VHH domain comprises an amino acid sequence set forth in SEQ ID NO: 544;
cn) the VHH domain comprises an amino acid sequence set forth in SEQ ID NO: 545;
co) the VHH domain comprises an amino acid sequence set forth in SEQ ID NO: 546;
cp) the VHH domain comprises an amino acid sequence set forth in SEQ ID NO: 547;
cq) the VHH domain comprises an amino acid sequence set forth in SEQ ID NO: 548;
cr) the VHH domain comprises an amino acid sequence set forth in SEQ ID NO: 549;
cs) the VHH domain comprises an amino acid sequence set forth in SEQ ID NO: 550;
ct) the VHH domain comprises an amino acid sequence set forth in SEQ ID NO: 551;
cu) the VHH domain comprises an amino acid sequence set forth in SEQ ID NO: 552;
cv) the VHH domain comprises an amino acid sequence set forth in SEQ ID NO: 553;
cw) the VHH domain comprises an amino acid sequence set forth in SEQ ID NO: 554;
cx) the VHH domain comprises an amino acid sequence set forth in SEQ ID NO: 555;
cy) the VHH domain comprises an amino acid sequence set forth in SEQ ID NO: 556;
cz) the VHH domain comprises an amino acid sequence set forth in SEQ ID NO: 557;
da) the VHH domain comprises an amino acid sequence set forth in SEQ ID NO: 558;
db) the VHH domain comprises an amino acid sequence set forth in SEQ ID NO: 559;
dc) the VHH domain comprises an amino acid sequence set forth in SEQ ID NO: 560;
dd) the VHH domain comprises an amino acid sequence set forth in SEQ ID NO: 561;
de) the VHH domain comprises an amino acid sequence set forth in SEQ ID NO: 562;
df) the VHH domain comprises an amino acid sequence set forth in SEQ ID NO: 563;
dg) the VHH domain comprises an amino acid sequence set forth in SEQ ID NO: 564;
dh) the VHH domain comprises an amino acid sequence set forth in SEQ ID NO: 565;
di) the VHH domain comprises an amino acid sequence set forth in SEQ ID NO: 566;
dj) the VHH domain comprises an amino acid sequence set forth in SEQ ID NO: 567;
dk) the VHH domain comprises an amino acid sequence set forth in SEQ ID NO: 568;
dl) the VHH domain comprises an amino acid sequence set forth in SEQ ID NO: 569;
dm) the VHH domain comprises an amino acid sequence set forth in SEQ ID NO: 570;
dn) the VHH domain comprises an amino acid sequence set forth in SEQ ID NO: 571;
do) the VHH domain comprises an amino acid sequence set forth in SEQ ID NO: 572;
dp) the VHH domain comprises an amino acid sequence set forth in SEQ ID NO: 573;
dq) the VHH domain comprises an amino acid sequence set forth in SEQ ID NO: 574;
dr) the VHH domain comprises an amino acid sequence set forth in SEQ ID NO: 575;
ds) the VHH domain comprises an amino acid sequence set forth in SEQ ID NO: 576;
dt) the VHH domain comprises an amino acid sequence set forth in SEQ ID NO: 577;
du) the VHH domain comprises an amino acid sequence set forth in SEQ ID NO: 578;
dv) the VHH domain comprises an amino acid sequence set forth in SEQ ID NO: 579;
dw) the VHH domain comprises an amino acid sequence set forth in SEQ ID NO: 580;
dx) the VHH domain comprises an amino acid sequence set forth in SEQ ID NO: 581;
dy) the VHH domain comprises an amino acid sequence set forth in SEQ ID NO: 582;
dz) the VHH domain comprises an amino acid sequence set forth in SEQ ID NO: 583;
ea) the VHH domain comprises an amino acid sequence set forth in SEQ ID NO: 584;
eb) the VHH domain comprises an amino acid sequence set forth in SEQ ID NO: 585;
ec) the VHH domain comprises an amino acid sequence set forth in SEQ ID NO: 586;
ed) the VHH domain comprises an amino acid sequence set forth in SEQ ID NO: 587;
ee) the VHH domain comprises an amino acid sequence set forth in SEQ ID NO: 588;
ef) the VHH domain comprises an amino acid sequence set forth in SEQ ID NO: 589;
eg) the VHH domain comprises an amino acid sequence set forth in SEQ ID NO: 590;
eh) the VHH domain comprises an amino acid sequence set forth in SEQ ID NO: 591;
ei) the VHH domain comprises an amino acid sequence set forth in SEQ ID NO: 592;
ej) the VHH domain comprises an amino acid sequence set forth in SEQ ID NO: 593;
ek) the VHH domain comprises an amino acid sequence set forth in SEQ ID NO: 594;
el) the VHH domain comprises an amino acid sequence set forth in SEQ ID NO: 595;
em) the VHH domain comprises an amino acid sequence set forth in SEQ ID NO: 596;
en) the VHH domain comprises an amino acid sequence set forth in SEQ ID NO: 597;
eo) the VHH domain comprises an amino acid sequence set forth in SEQ ID NO: 598;
ep) the VHH domain comprises an amino acid sequence set forth in SEQ ID NO: 599;
eq) the VHH domain comprises an amino acid sequence set forth in SEQ ID NO: 600;
er) the VHH domain comprises an amino acid sequence set forth in SEQ ID NO: 601;
es) the VHH domain comprises an amino acid sequence set forth in SEQ ID NO: 602;
et) the VHH domain comprises an amino acid sequence set forth in SEQ ID NO: 603; or
eu) the VHH domain comprises an amino acid sequence set forth in SEQ ID NO: 604.

3. The antibody or antigen-binding fragment thereof of claim 1, wherein the antibody or antigen-binding fragment thereof is a chimeric or humanized antibody or antigen-binding fragment thereof.

4. The antibody or antigen-binding fragment thereof of claim 1, wherein the antibody or antigen-binding fragment thereof is a monoclonal antibody or antigen-binding fragment thereof.

5. The antibody or antigen-binding fragment thereof of claim 1, wherein the antibody or antigen-binding fragment thereof is a bispecific antibody.

6. The antibody or antigen-binding fragment thereof of claim 5, wherein the bispecific antibody comprises an antigen binding domain comprising binding affinity to a tumor associated antigen (TAA).

7. The antibody or antigen-binding fragment thereof of claim 1, wherein the antibody or antigen-binding fragment thereof is operatively linked to a CH1 domain and/or a CL domain.

8. The antibody or antigen-binding fragment thereof of claim 6, wherein the antibody with binding affinity to CD28 is operatively linked to a CH1 domain and the antigen binding domain with binding affinity to the TAA is operatively linked to a CL domain.

9. The antibody or antigen-binding fragment thereof of claim 6, wherein the antibody with binding affinity to CD28 is operatively linked to a CL domain and the antigen binding domain with binding affinity to the TAA is operatively linked to a CH1 domain.

10. The antibody or antigen-binding fragment thereof of claim 1, wherein the antibody or antigen-binding fragment thereof is operatively linked to an Fc region.

11. The antibody or antigen-binding fragment thereof of claim 10, wherein the Fc region is a human IgG1 Fc region.

12. The antibody or antigen-binding fragment thereof of claim 1, comprising an antagonistic antibody or antigen-binding fragment thereof.

13. An isolated nucleic acid molecule encoding the antibody or antigen-binding fragment thereof of claim 1.

14. An expression vector comprising the nucleic acid molecule of claim 13.

15. A host cell comprising the expression vector of claim 14.

16. A method for inhibiting CD28 activity in a subject, comprising administering to a subject the antibody or antigen-binding fragment thereof according to claim 1, thereby inhibiting CD28 activity in the subject.

17. A method for treating diseases associated with CD28 activity in a subject, comprising administering to a subject in need thereof the antibody or antigen-binding fragment thereof according to claim 1.

18. The method of claim 17, wherein the disease is an autoimmune disease.

19. The method of claim 17, wherein the disease is cancer.
